(19) United States
(12) Patent Application Publication
(10) Pub. No.: US 2009/0105301 A1
(43) Pub. Date: Apr. 23, 2009

(54) 9A-SUBSTITUTED AZALIDES FOR THE TREATMENT OF MALARIA

(76) Inventors: Sulejman Alihodzic, Zagreb (HR); Andrea Fajdetic, Zagreb (HR); Anton Hutinec, Zagreb (HR); Zrinka Ivezic, Zagreb (HR); Nedjeljko Kujundzic, Zagreb (HR); Renata Rupcic, Zagreb (HR)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/299,042

(22) PCT Filed: May 1, 2007
(86) PCT No.: PCT/IB07/01216

§ 371 Date: Dec. 22, 2008

Related U.S. Application Data
(60) Provisional application No. 60/797,505, filed on May 3, 2006.

(51) Int. Cl. A61K 31/4709; C07D 267/00; A61K 31/395

(52) U.S. Cl. 514/314; 540/467; 514/450

(57) ABSTRACT

The present invention relates to novel 9a-substituted azalides having antimalarial activity. More particularly, the invention relates to 9a-substituted 9a-aza-9-deoxo-9-dihydro-9a-homoerythromycin A, 3-O-decladinosyl-9a-aza-9-deoxo-9-dihydro-9a-homoerythromycin A and 3-O-decladinosyl-5-O-dedesosaminyl-9a-aza-9-deoxo-9-dihydro-9a-homoerythromycin A compounds having antimalarial activity, to the method of preparation, to the method of use, and to pharmaceutically acceptable derivatives thereof having antimalarial activity.

### FIELD OF THE INVENTION

The present invention relates to novel 9a-substituted azalides having antimalarial activity. More particularly, the invention relates to 9a-substituted 9a-aza-9-deoxo-9-dihydro-9a-homoerythromycin A, 3-O-decladinosyl-9a-aza-9-deoxo-9-dihydro-9a-homoerythromycin A and 3-O-decladinosyl-5-O-dedesosaminyl-9a-aza-9-deoxo-9-dihydro-9a-homoerythromycin A compounds having antimalarial activity, to methods for their preparation, to their use as therapeutic agents, and to pharmaceutically acceptable derivatives thereof having antimalarial activity.

### BACKGROUND OF THE INVENTION

Malaria is a serious infection. 200 to 300 million people are infected with malaria and two to three million people die from malaria every year. The disease is caused by a parasite (a protozoa of the Plasmodia genus), which is transmitted by the female Anopheles mosquito. There are four parasites that can affect humans, Plasmodium falciparum, P. vivax, P. ovale, and P. malariae. A distinction is drawn between Malaria tropica (caused by Plasmodium falciparum), Malaria tertiana (caused by Plasmodium vivax or Plasmodium ovale) and Malaria quartana (caused by Plasmodium malariae). Malaria tropica is the most severe form of the disease, and is characterized by severe constitutional symptoms, and sometimes causes death.

Malaria is characterized by attacks of chills, fever, and sweating, occurring at intervals which depend on the time required for development of a new generation of parasites in the body. After recovery from the acute attack, the disease has a tendency to become chronic, with occasional relapses. The disease is prevalent in tropical and subtropical areas of the world including the Amazon region of Brazil, East and Southern Africa and Southeast Asia. The emergence of a malaria parasite resistant to chloroquine, which is a drug used extensively in the treatment of malaria, has become a serious problem, and therefore, there is an urgent need to develop an effective remedy. Also, attempts to develop a malaria vaccine have failed to date. This compounds the urgent need to find an alternative drug-based approach to treating malaria.

Drugs of diverse chemical classes, such as chloroquine, mefloquine, halofantrine, and artemisinin, atovaquone/proguanil (Malarone™), doxycycline, and primaquine have been developed for the treatment of malaria. However, while marginally successful against some strains of malaria, most strains of malaria appear to have developed resistance not only to individual drugs but also to multiple combinations of drugs. Drugs which worked initially become totally ineffective after a period of time. An initial period of remission is often followed by a period during which nothing seems to be effective against the disease. This is known as multiple drug resistance, and it remains an issue in antimalarial drug development efforts. A malarial parasite which initially responds to treatment by one or more drugs becomes resistant to treatment not only using the drugs previously used, but many other antimalarial drugs. This further underscores the urgent necessity to find new compounds which show good efficacy against malaria and minimal toxicity.

In recent years several reports indicated that macrolides have potential for prophylactic as well as therapeutic use against malaria. Midecamycinin was studied in 1989 in two infectious models using Plasmodium berghei and Plasmodium yoelii nigeriensis (mouse) and Plasmodium cynomolgi (rhesus monkey) [S. K. Puri and G. P. Duti, Chemotherap. 35 (1989) 187]. In both mouse models, the macrolide midecamycinin was active. The doses for Plasmodium berghei infection were significantly lower than for Plasmodium yoelii nigeriensis. In the monkey model, no efficacy was noted. In other investigations the animal model was challenged with azithromycin [S. K. Puri and N. Singh, Exp. Parasitol. 94 (2000) 8]. The dose regimen of 25-50 mg/kg reflects the same dose used for antibacterial treatment. Azithromycin worked in prophylactic and therapeutic dosing and in contrast to midecamycinin azithromycin was active also in the monkey model.

The efficacy of azithromycin in treating malarial infections was studied in Gambia [S. T. Sadiq et al, Lancet 346 (1995), 881]. Children undergoing therapy for trachoma (Azithromycin is highly effective against C. trachomatis) were also examined for signs of malaria prophylaxis or therapeutic effects. A clear improvement of various indicators of malaria infection suggested a significant therapeutic benefit of azithromycin. The prophylactic efficacy of azithromycin was confirmed in Kenya [S. L. Anderson et al., Ann. Intern. Med. 123 (1995) 771]. A significant protection in adult volunteers was achieved with a better prophylaxis obtained through use of a daily dosing scheme of 250 mg versus a weekly regimen of 1000 mg. Also, in a double-blind, placebo-controlled trial with azithromycin in Irian Jaya in Indonesia [W. R. Taylor et al., Clin. Infect Dis. 28 (1999) 74], the prophylactic efficacy in azithromycin treated non-immune patients was 71.6% for Plasmodium falciparum and 98.9% for Plasmodium vivax as compared to controls.

### SUMMARY OF THE INVENTION

Use of 9a-substituted 9a-aza-9-deoxo-9-dihydro-9a-homoerythromycin A, 3-O-decladinosyl-9a-aza-9-deoxo-9-dihydro-9a-homoerythromycin A and 3-O-decladinosyl-5-O-dedesosaminyl-9a-aza-9-deoxo-9-dihydro-9a-homoerythromycin A compounds represented by the Formula (I):

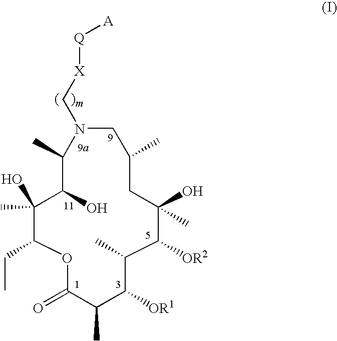

wherein
R¹ represents H or a α-L-cladinosyl group of formula (II)

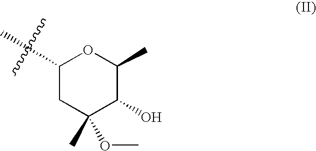

R² represents H or a β-D-desosaminyl group of formula (III)

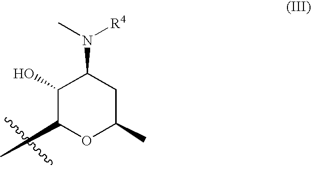

provided that when R² is H then R¹ is also H;
X represents NR³ or NHC(═O) or C(═O)NH;
R³ represents H or linear or branched C₁₋₄alkyl;
R⁴ represents H or linear or branched C₁₋₄alkyl;
Q represents
a) single bond,
b) C₁₋₄alkylene linear or branched which is unsubstituted or substituted,
c) C₂₋₄alkenylene;
   A represents
a) aryl is mono-, bicyclic or tricyclic carbocyclic ring system having at least one aromatic ring which is unsubstituted or substituted by 1-4 groups selected from unsubstituted or substituted C₁₋₄ alkyl, unsubstituted or substituted C₃₋₆ cycloalkyl, halogen, OH, NO₂, C₁₋₄ alkyloxy, C₃₋₆ cycloalkyloxy, C₁₋₄ alkylamino, C₁₋₄ dialkylamino, C₃₋₆ cycloalkylamino;
b) 3-14 membered heterocycle, which is monocyclic, bicyclic or tricyclic ring any of which is saturated, unsaturated or aromatic containing 1 to 4 heteroatoms selected from nitrogen (unsubstituted or substituted by H or C₁₋₄ alkyl), oxygen and sulphur, unsubstituted or substituted on 1-3 ring carbon atoms by groups independently selected from unsubstituted or substituted C₁₋₄ alkyl, unsubstituted or substituted C₃₋₆ cycloalkyl, halogen, OH, NO₂, C₁₋₄ alkyloxy, C₃₋₆ cycloalkyloxy, C₁₋₄ alkylamino, C₁₋₄ dialkylamino, C₃₋₆ cycloalkylamino;
   m is an integer from 2 to 4;
   or pharmaceutically acceptable derivatives thereof
   in the manufacture of a medicament for the treatment and/or prophylaxis of malaria.

Novel 9a-substituted 9a-aza-9-deoxo-9-dihydro-9a-homoerythromycin A, 3-O-decladinosyl-9a-aza-9-deoxo-9-dihydro-9a-homoerythromycin A and 3-O-decladinosyl-5-O-dedesosaminyl-9a-aza-9-deoxo-9-dihydro-9a-homoerythromycin A compounds of Formula (I) are represented by Formula (Ia):

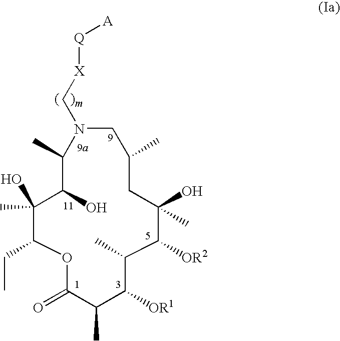

wherein
R¹ represents H or a α-L-cladinosyl group of formula (II)

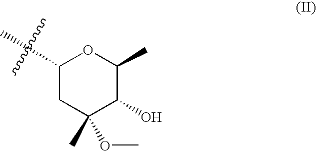

R² represents H or a β-D-desosaminyl group of formula (III)

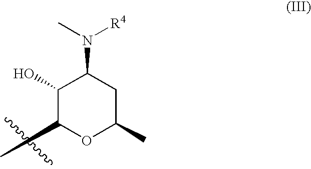

provided that when R² is H then R¹ is also H;
X represents NR³ or NHC(═O) or C(═O)NH;
R³ represents H or linear or branched C₁₋₄alkyl;
R⁴ represents H or linear or branched C₁₋₄alkyl;
Q represents
a) single bond,
b) C₁₋₄alkylene linear or branched which is unsubstituted or substituted,
c) C₂₋₄alkenylene;
   A represents
a) aryl is mono-, bicyclic or tricyclic carbocyclic ring system having at least one aromatic ring which is unsubstituted or substituted by 1-4 groups selected from unsubstituted or substituted C₁₋₄ alkyl, unsubstituted or substituted C₃₋₆ cycloalkyl, halogen, OH, NO₂, C₁₋₄ alkyloxy, C₃₋₆ cycloalkyloxy, C₁₋₄ alkylamino, C₁₋₄ dialkylamino, C₃₋₆ cycloalkylamino;
b) 3-14 membered heterocycle, which is monocyclic, bicyclic or tricyclic ring any of which is saturated, unsaturated or aromatic containing 1 to 4 heteroatoms selected from nitrogen (unsubstituted or substituted by H or C₁₋₄ alkyl), oxygen and sulphur, unsubstituted or substituted on 1-3 ring carbon atoms by groups independently selected from unsubstituted or substituted C₁₋₄ alkyl, unsubstituted or substituted C₃₋₆ cycloalkyl, halogen, OH, NO₂, C₁₋₄ alkyloxy, C₃₋₆ cycloalkyloxy, C₁₋₄ alkylamino, C₁₋₄ dialkylamino, C₃₋₆ cycloalkylamino;
   m is an integer from 2 to 4;
   provided that when R² represents a β-D-desosaminyl group of formula (III),
   X represents NR³,
   R³ represents H or C₁₋₃alkyl,
   m represents 2 or 3,
   Q represents linear unsubstituted C₁₋₄alkylene and A represents unsubstituted or substituted phenyl or unsubstituted or substituted heteroaryl with five or six members containing from 1 to 3 atoms selected from nitrogen, oxygen and sulphur, then R⁴ represents linear or branched C₄alkyl;
   provided that A cannot represent a nonsteroidal subunit derived from a nonsteroidal anti-inflammatory drug (NSAID);
   or pharmaceutically acceptable derivatives thereof.

The present invention also relates to pharmaceutical compositions comprising the Formula I compounds and pharmaceutically acceptable derivatives thereof.

Furthermore, the present invention also relates to methods of treating malarial diseases comprising administration of a therapeutically effective amount of a compound of Formula I to a patient in need thereof. Moreover, novel compounds of the present invention may exhibit good potency against plasmodia, especially against multiresistant plasmodial species.

According to another aspect of the invention there is provided at least one compound of Formula I or a pharmaceutically acceptable derivative thereof for use in human or veterinary medical therapy.

In another aspect of the invention there is provided the use of at least one compound of Formula I or a pharmaceutically acceptable derivative thereof in the manufacture of a medicament for the treatment of malaria.

The present invention is also directed to compositions containing one or more of the foregoing compounds in an amount effective for therapeutic and/or prophylactic treatment of malaria in a subject in need of such treatment.

The present invention is also directed to the methods for using the compounds of Formula I in the prophylaxis of malaria or the treatment of subjects exposed to the malaria parasites.

### DETAILED DESCRIPTION OF THE INVENTION

In one particular embodiment, the present invention is directed to the use of 9a-substituted 9a-aza-9-deoxo-9-dihydro-9a-homoerythromycin A, 3-O-decladinosyl-9a-aza-9-deoxo-9-dihydro-9a-homoerythromycin A and 3-O-decladinosyl-5-O-dedesosaminyl-9a-aza-9-deoxo-9-dihydro-9a-homoerythromycin A compounds represented by the Formula (I):

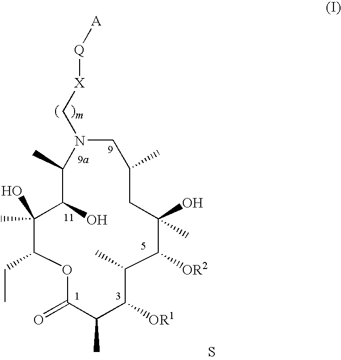

wherein
R¹ represents H or a α-L-cladinosyl group of formula (II)

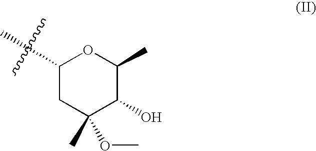

R² represents H or a β-D-desosaminyl group of formula (III)

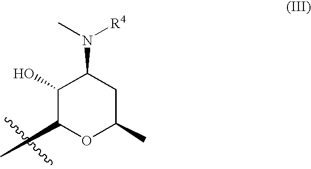

provided that when R² is H then R¹ is also H;
X represents NR³ or NHC(═O) or C(═O)NH;
R³ represents H or linear or branched C₁₋₄alkyl;
R⁴ represents H or linear or branched C₁₋₄alkyl;
Q represents
a) single bond,
b) C₁₋₄alkylene linear or branched which is unsubstituted or substituted,
c) C₂₋₄alkenylene;
   A represents
a) aryl is mono-, bicyclic or tricyclic carbocyclic ring system having at least one aromatic ring which is unsubstituted or substituted by 1-4 groups selected from unsubstituted or substituted C₁₋₄ alkyl, unsubstituted or substituted C₃₋₆ cycloalkyl, halogen, OH, NO₂, C₁₋₄ alkyloxy, C₃₋₆ cycloalkyloxy, C₁₋₄ alkylamino, C₁₋₄ dialkylamino, C₃₋₆ cycloalkylamino;
b) 3-14 membered heterocycle, which is monocyclic, bicyclic or tricyclic ring any of which is saturated, unsaturated or aromatic containing 1 to 4 heteroatoms selected from nitrogen (unsubstituted or substituted by H or C₁₋₄ alkyl), oxygen and sulphur, unsubstituted or substituted on 1-3 ring carbon atoms by groups independently selected from unsubstituted or substituted C₁₋₄ alkyl, unsubstituted or substituted C₃₋₆ cycloalkyl, halogen, OH, NO₂, C₁₋₄ alkyloxy, C₃₋₆ cycloalkyloxy, C₁₋₄ alkylamino, C₁₋₄ dialkylamino, C₃₋₆ cycloalkylamino;
   m is an integer from 2 to 4;
   or pharmaceutically acceptable derivatives thereof
   in the manufacture of a medicament for the treatment and/or prophylaxis of malaria.

In a further embodiment the present invention relates to novel 9a-substituted 9a-aza-9-deoxo-9-dihydro-9a-homoerythromycin A, 3-O-decladinosyl-9a-aza-9-deoxo-9-dihydro-9a-homoerythromycin A and 3-O-decladinosyl-5-O-dedesosaminyl-9a-aza-9-deoxo-9-dihydro-9a-homoerythromycin A compounds of Formula (I) which are represented by Formula (Ia):

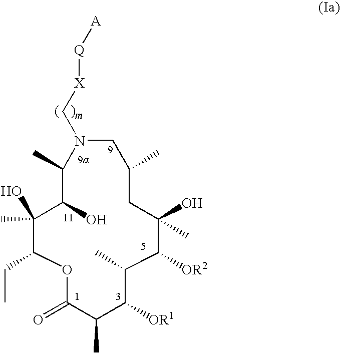

wherein
R¹ represents H or a α-L-cladinosyl group of formula (II)

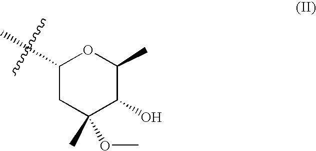

R² represents H or a β-D-desosaminyl group of formula (III)

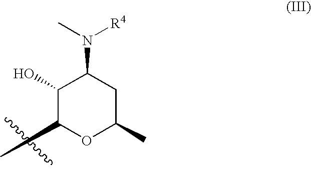

provided that when R² is H then R¹ is also H;
X represents NR³ or NHC(═O) or C(═O)NH;
R³ represents H or linear or branched C₁₋₄alkyl;
R⁴ represents H or linear or branched C₁₋₄alkyl;
Q represents
a) single bond,
b) C₁₋₄alkylene linear or branched which is unsubstituted or substituted,
c) C₂₋₄alkenylene;
   A represents
a) aryl is mono-, bicyclic or tricyclic carbocyclic ring system having at least one aromatic ring which is unsubstituted or substituted by 1-4 groups selected from unsubstituted or substituted C₁₋₄ alkyl, unsubstituted or substituted C₃₋₆ cycloalkyl, halogen, OH, NO₂, C₁₋₄ alkyloxy, C₃₋₆ cycloalkyloxy, C₁₋₄ alkylamino, C₁₋₄ dialkylamino, C₃₋₆ cycloalkylamino;
b) 3-14 membered heterocycle, which is monocyclic, bicyclic or tricyclic ring any of which is saturated, unsaturated or aromatic containing 1 to 4 heteroatoms selected from nitrogen (unsubstituted or substituted by H or C₁₋₄ alkyl), oxygen and sulphur, unsubstituted or substituted on 1-3 ring carbon atoms by groups independently selected from unsubstituted or substituted C₁₋₄ alkyl, unsubstituted or substituted C₃₋₆ cycloalkyl, halogen, OH, NO₂, C₁₋₄ alkyloxy, C₃₋₆ cycloalkyloxy, C₁₋₄ alkylamino, C₁₋₄ dialkylamino, C₃₋₆ cycloalkylamino;
   m is an integer from 2 to 4;
   provided that when R² represents a β-D-desosaminyl group of formula (III),
   X represents NR³,
   R³ represents H or C₁₋₃alkyl,
   m represents 2 or 3,
   Q represents linear unsubstituted C₁₋₄alkylene and A represents unsubstituted or substituted phenyl or unsubstituted or substituted heteroaryl with five or six members containing from 1 to 3 atoms selected from nitrogen, oxygen and sulphur then R⁴ represents linear or branched C₄alkyl;
   provided that A cannot represent a nonsteroidal subunit derived from a nonsteroidal anti-inflammatory drug (NSAID);
   or pharmaceutically acceptable derivatives thereof.

In one aspect the present invention provides use of the compounds of Examples 1 to 79 or pharmaceutically acceptable derivatives thereof.

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). Preferably, as used herein, the term “pharmaceutically acceptable” means approved by a regulatory agency of the Federal or a state government or listed in the U.S. Pharmacopoeia or other generally recognized pharmacopoeia for use in mammals, and more particularly in humans.

The term “carrier” applied to pharmaceutical compositions of the invention refers to a diluent, excipient, or vehicle with which an active compound is administered. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Suitable pharmaceutical carriers are described in “Remington's Pharmaceutical Sciences” by E. W. Martin, 18th Edition, incorporated by reference. Particularly preferred for the present invention are carriers suitable for immediate-release, i.e., release of most or all of the active ingredient over a short period of time, such as 60 minutes or less, and make rapid absorption of the drug possible.

The term “pharmaceutically acceptable derivative” as used herein means any pharmaceutically acceptable salt, solvate or prodrug, e.g. ester, of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5^{th} Edition, Vol 1: Principles and Practice, which is incorporated herein by reference to the extent of teaching such derivatives. In one aspect pharmaceutically acceptable derivatives are salts, solvates, esters, carbamates and phosphate esters. In another aspect pharmaceutically acceptable derivatives are salts, solvates and esters. In a further aspect pharmaceutically acceptable derivatives are salts and esters.

The compounds of the present invention may be in the form of and/or may be administered as a pharmaceutically acceptable salt. For a review on suitable salts see Berge et al., J. Pharm. Sci., 66 (1977) 1-19.

Typically, a pharmaceutical acceptable salt may be readily prepared by using a desired acid. The salt may precipitate from solution and be collected by filtration or may be recovered by evaporation of the solvent. For example, an aqueous solution of an acid such as hydrochloric acid may be added to an aqueous suspension of a compound of formula (I) and the resulting mixture evaporated to dryness (lyophilised) to obtain the acid addition salt as a solid. Alternatively, a compound of formula (I) may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

Suitable addition salts are formed from inorganic or organic acids which form non-toxic salts and examples are hydrochloride, hydrobromide, hydroiodide, sulphate, bisulphate, nitrate, phosphate, hydrogen phosphate, acetate, trifluoroacetate, maleate, malate, fumarate, lactate, tartrate, citrate, formate, gluconate, succinate, pyruvate, oxalate, oxaloacetate, trifluoroacetate, saccharate, benzoate, alkyl or aryl sulphonates (eg methanesulphonate, ethanesulphonate, benzenesulphonate or p-toluenesulphonate) and isethionate. Representative examples include trifluoroacetate and formate salts, for example the bis or tris trifluoroacetate salts and the mono or diformate salts, in particular the tris or bis trifluoroacetate salt and the monoformate salt.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compounds of the invention are within the scope of the invention. The salts of the compound of Formula (I) may form solvates (e.g. hydrates) and the invention also includes all such solvates.

The term “prodrug” as used herein means a compound which is converted within the body, e.g. by hydrolysis in the blood, into its active form that has medical effects. Pharmaceutically acceptable prodrugs are described in T. Higuchi and V. Stella, “Prodrugs as Novel Delivery Systems”, Vol. 14 of the A.C.S. Symposium Series, Edward B. Roche, ed., “Bioreversible Carriers in Drug Design”, American Pharmaceutical Association and Pergamon Press, 1987, and in D. Fleisher, S. Ramon and H. Barbra “Improved oral drug delivery: solubility limitations overcome by the use of prodrugs”, Advanced Drug Delivery Reviews 19 (2) (1996) 115-130, each of which are incorporated herein by reference.

Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol, sulfhydryl and amine functional groups of the compounds of structure (I). Further, in the case of a carboxylic acid (—COOH), esters may be employed, such as methyl esters, ethyl esters, and the like. Esters may be active in their own right and/or be hydrolysable under in vivo conditions in the human body. Suitable pharmaceutically acceptable in vivo hydrolysable ester groups include those which break down readily in the human body to leave the parent acid or its salt.

References hereinafter to a compound according to the invention include both compounds of Formula (I) and their pharmaceutically acceptable derivatives.

With regard to stereoisomers, the compounds of Formula (I) have more than one asymmetric carbon atom. In the general Formula (I) as drawn, the solid wedge shaped bond indicates that the bond is above the plane of the paper. The broken bond indicates that the bond is below the plane of the paper.

It will be appreciated that the substituents on the macrolide may also have one or more asymmetric carbon atoms. Thus, the compounds of Formula (I) may occur as individual enantiomers or diastereomers. All such isomeric forms are included within the present invention, including mixtures thereof.

Where a compound of the invention contains an alkenyl group, cis (Z) and trans (E) isomerism may also occur. The present invention includes the individual stereoisomers of the compounds of the invention and, where appropriate, the individual stereoisomeric forms thereof, together with mixtures.

Separation of diastereoisomers may be achieved by conventional techniques, e.g. by fractional crystallisation, chromatography or H.P.L.C. An individual stereoisomer may also be prepared from a corresponding optically pure intermediate or by resolution, such as H.P.L.C., of the corresponding mixture using a suitable chiral support or by fractional crystallisation of the diastereoisomeric salts formed by reaction of the corresponding mixture with a suitable optically active acid or base, as appropriate.

The compounds of Formula (I) may be in crystalline or amorphous form. Furthermore, some of the crystalline forms of the compounds of Formula (I) may exist as polymorphs, which are included in the present invention.

In one aspect of the invention R¹ represents a α-L-cladinosyl group of formula (II) and R² represents a β-D-desosaminyl group of formula (III).

In one aspect of the invention R¹ represents H and R² represents a β-D-desosaminyl group of formula (III).

In one aspect of the invention R¹ represents H and R² represents H.

In one aspect of the invention X represents NR³ and R³ represents H.

In one aspect of the invention X represents NR³ where R³ represents linear C₁₋₄alkyl. In a further aspect R³ represents methyl.

In one aspect of the invention X represents NH or NCH₃.

In one aspect of the invention X represents NHC(═O) or C(═O)NH.

In one aspect of the invention R⁴ represents linear C₁₋₄alkyl. In a further aspect R⁴ represents methyl.

In one aspect of the invention R⁴ represents H.

In one aspect of the invention Q represents a single bond.

In one aspect of the invention Q represents C₁₋₄alkylene such as methylene, ethylene, propylene or butylene.

In one aspect of the invention Q represents C₁₋₄alkylene, such as methylene, substituted with C₁₋₄alkyl, such as methyl.

In one aspect of the invention Q represents C₁₋₄alkylene, such as ethylene, substituted with NC(═O)—C₁-C₄-alkyl, such as NHC(═O)CH₃.

In one aspect of the invention Q represents C₁₋₄alkylene, such as propylene, substituted with oxo.

In one aspect of the invention Q represents C₂₋₄alkenylene, such as ethenylene.

In one aspect of the invention A is mono- or bicyclic unsubstituted or substituted aryl such as phenyl or naphthyl.

In one aspect of the invention A is unsubstituted or substituted 3 to 14 membered heterocycle.

In one aspect of the invention A is a quinoline derived moiety:

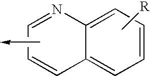

R is H or halogen

In one aspect of the invention, when A is a quinoline derived moiety, the quinoline derived moiety is linked to the reminder of the molecule through the 2, 3 or 4 position.

In one aspect of the invention, when A is a quinoline derived moiety, the quinoline derived moiety is substituted with halogen such as chlorine. In a further aspect of the invention the halogen is attached to the quinoline moiety at the 7 position.

In one aspect of the invention, when A is a quinoline derived moiety, the first ring of the quinoline derived moiety is saturated, such as 1,2,3,4-tetrahydroquinolinyl.

In one aspect of the invention A is a naphthalene derived moiety:

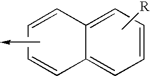

R is H or C₁₋₄alkyloxy and may be attached to either ring.

In one aspect of the invention when A is a naphthalene derived moiety, the naphthalene derived moiety is linked to the reminder of the molecule through the 1 or 2 position.

In one aspect of the invention when A is a naphthalene derived moiety, the naphthalene derived moiety is substituted with C₁₋₄alkyloxy, such as methyloxy or ethyloxy.

In one aspect of the invention A is a pyridine derived moiety:

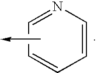

In one aspect of the invention, when A is a pyridine derived moiety, the pyridine derived moiety is linked to the reminder of the molecule through the 2, 3 or 4 position.

In one aspect of the invention A is a phenyl derived moiety:

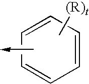

R is H, C₁₋₄alkyloxy, halogen or NO₂
t is 1 to 4.

In one aspect of the invention, when A is a phenyl derived moiety, the phenyl derived moiety is substituted with one to four groups selected from C₁₋₄ alkyloxy, such as methyloxy or ethyloxy, halogen such as fluoro, chloro or bromo, and NO₂.

In one aspect of the invention A is a purine derived moiety:

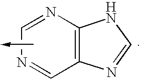

In a further aspect A is 1H-purin-6-yl.

In one aspect of the invention A is an oxazole derived moiety:

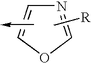

R is C₁₋₄alkyl.

In a further aspect of the invention A is 4-methyl-1,3-oxazole-5-yl.

In one aspect of the invention “m” represents 2 or 3.

In one aspect the present invention is directed to compounds of Formula (I) represented by Formula (Ib)

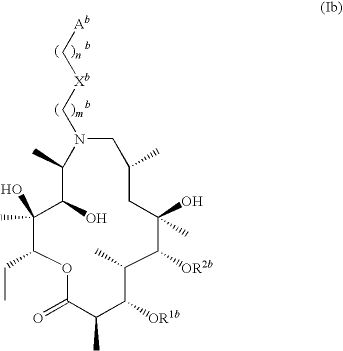

wherein
R^{1b} represents H or a cladinosyl group of formula (IIb)

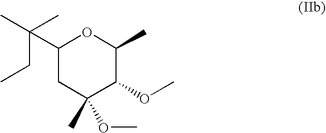

R^{2b} represents H or a desozaminyl group of formula (IIIb)

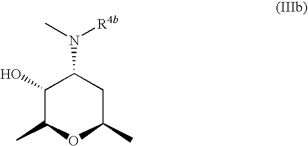

X^{b} represents NR^{3b} or NHC(═O) or C(═O)NH;
R^{3b} represents H or linear or branched C₁₋₄alkyl (preferably methyl or ethyl);
R^{4b} represents H or linear or branched C₁₋₄alkyl (preferably methyl or ethyl);
A^{b} represents
a) aryl which is unsubstituted or substituted by 1-3 groups selected from unsubstituted or substituted C₁₋₄ alkyl (preferably methyl or ethyl), unsubstituted or substituted C₃₋₆ cycloalkyl (preferably cyclopropyl or cyclohexyl), halogen (preferably fluoro, chloro or bromo), OH, C₁₋₄ alkyloxy (preferably methyloxy or ethyoxy), C₃₋₆ cycloalkyloxy (preferably cyclopropyloxy or cyclohexyloxy), C₁₋₄ alkylamino (preferably methylamino or ethylamino), C₁₋₄ dialkylamino (preferably dimethylamino or diethylamino), C₃₋₆ cycloalkylamino (preferably cyclopropylamino or cyclohexylamino);
b) 3-14 membered heterocycle containing 1 to 4 heteroatoms selected from the nitrogen, oxygen and sulphur optionally substituted by 1-3 groups selected from unsubstituted or substituted C₁₋₄ alkyl (preferably methyl or ethyl), unsubstituted or substituted C₃₋₆ cycloalkyl (preferably cyclopropyl or cyclohexyl), halogen (preferably fluoro, chloro or bromo), OH, C₁₋₄ alkyloxy (preferably methyloxy or ethyoxy), C₃₋₆ cycloalkyloxy (preferably cyclopropyloxy or cyclohexyloxy), C₁₋₄ alkylamino (preferably methylamino or ethylamino), C₁₋₄ dialkylamino (preferably dimethylamino or diethylamino), C₃₋₆ cycloalkylamino (preferably cyclopropylamino or cyclohexylamino);
   m^{b} is an integer from 2 to 4;
   n^{b} is an integer from 0 to 4;
   provided that A^{b} cannot represent unsubstituted phenyl or unsubstituted heteroaryl with five or six members containing from 1 to 3 atoms selected from nitrogen, oxygen and sulphur when R^{1b} represents H, R^{2b} represent desosaminyl group of formula (IIIb), R^{4b} represents linear or branched C₁₋₄alkyl and X^{b} represents NR^{3b};
   or to pharmaceutically acceptable derivatives thereof.

It will be understood that the present invention covers all combinations of aspects, suitable, convenient and preferred groups described herein.

The term “NSAID” can represent a nonsteroidal anti-inflammatory subunit, i.e., a moiety of a nonsteroidal antiinflammatory drug (NSAID). Suitable NSAIDs include, but are not limited to, drugs which inhibit cyclooxygenase, the enzyme responsible for the biosyntheses of the prostaglandins and certain autocoid inhibitors, including inhibitors of the various isoenzymes of cyclooxygenase (including, but not limited to, cyclooxygenase-1 and -2), and drugs which inhibit both cyclooxygenase and lipoxygenase; for example the commercially available NSAIDs aceclofenac, acemetacin, acetaminophen, acetaminosalol, acetyl-salicylic acid, acetyl-salicylic-2-amino-4-picoline-acid, 5-aminoacetylsalicylic acid, alclofenac, aminoprofen, amfenac, ampyrone, ampiroxicam, anileridine, bendazac, benoxaprofen, bermoprofen, α-bisabolol, bromfenac, 5-bromosalicylic acid acetate, bromosaligenin, bucloxic acid, butibufen, carprofen, celecoxib, cromoglycate, cinmetacin, clidanac, clopirac, sodium diclofenac, diflunisal, ditazol, droxicam, enfenamic acid, etodolac, etofenamate, felbinac, fenbufen, fenclozic acid, fendosal, fenoprofen, fentiazac, fepradinol, flufenac, flufenamic acid, flunixin, flunoxaprofen, flurbiprofen, glutametacin, glycol salicylate, ibufenac, ibuprofen, ibuproxam, indomethacin, indoprofen, isofezolac, isoxepac, isoxicam, ketoprofen, ketorolac, lornoxicam, loxoprofen, meclofenamic acid, mefenamic acid, meloxicam, mesalamine, metiazinic acid, mofezolac, montelukast, mycophenolic acid, nabumetone, naproxen, niflumic acid, nimesulide, olsalazine, oxaceprol, oxaprozin, oxyphenbutazone, paracetamol, parsalmide, perisoxal, phenyl-acethyl-salicylate, phenylbutazone, phenylsalicylate, pirazolac, piroxicam, pirprofen, pranoprofen, protizinic acid, reserveratol, salacetamide, salicylamide, salicylamide-O-acetic acid, salicylsulphuric acid, salicin, salicylamide, salsalate, sulindac, suprofen, succibutazone, tamoxifen, tenoxicam, theophylline, tiaprofenic acid, tiaramide, ticlopridine, tinoridine, tolfenamic acid, tolmetin, tropesin, xenbucin, ximoprofen, zaltoprofen, zomepirac, tomoxiprole, zafirlukast and cyclosporine. Additional NSAID genera and particular NSAID compounds are disclosed in U.S. Pat. No. 6,297,260, incorporated entirely by reference (especially in the generic formulas of its claim 1 and the recitation of specific list of NSAID's contained therein and in claim 3), and thiazulidene NSAIDs disclosed in International Patent Application WO 01/87890, incorporated herein by reference in its entirety. Preferred are flufenamic acid, flunixin and celecoxib.

The term “C₁-C₄alkyl” as used herein, refers to saturated, straight or branched-chain hydrocarbon radicals containing between one and four carbon atoms. Examples of “C₁-C₄alkyl” radicals include; methyl, ethyl, propyl, isopropyl, n-butyl, isobutyl, tert-butyl.

The term “substituted alkyl” as used herein, refers to a “C₁-C₄ alkyl” group as previously defined, substituted by independent replacement of one, two, or three of the hydrogen atoms thereon with substituents including, but not limited to: halogen (such as fluoro, chloro or bromo); OH; oxo; C₁-C₄ alkylamino (such as N-methylamino or N-ethylamino); —NC(O)—C₁-C₄-alkyl (such as NC(O)-methyl); C₁-C₄-alkylamino (such as dimethylamino, diethylamino or di-isopropylamino); C₁-C₄ alkyloxy (such as methoxy or ethoxy); C₃-C₆ cycloalkyloxy (such as cyclopropoxy or cyclohexyloxy).

The term “alkyloxy” or “alkoxy”, as used herein, refers to a straight or branched chain C₁₋₄alkyl group, as previously defined, attached to the parent molecular moiety through an oxygen atom containing the specified number of carbon atoms. For example, C₁₋₄alkoxy means a straight or branched alkoxy containing at least 1, and at most 4, carbon atoms. Examples of “alkoxy” as used herein include, but are not limited to, methoxy, ethoxy, propoxy, prop-2-oxy, butoxy, but-2-oxy, 2-methylprop-1-oxy and 2-methylprop-2-oxy.

The term “C₁₋₄alkylene” as used herein refers to a linear or branched saturated hydrocarbon linker group which may be unsubstituted or substituted by C₁₋₄alkyl (such as methyl), —NHC(═O)CH₃, or oxo. Examples of such groups include methylene, methylmethylene, ethylene, propylene and butylene and the like.

The term “C₂₋₄alkenylene” as used herein refers to a linear or branched hydrocarbon linker group containing one or more carbon-carbon double bonds. Examples of alkenylene groups include ethenylene and propenylene and the like, suitably ethenylene.

The term “halogen” refers to a fluorine, chlorine, bromine or iodine atom.

The term “aryl”, as used herein, refers to a mono-, bicyclic or tricyclic carbocyclic ring system having at least one aromatic rings including, but not limited to, phenyl, azulenyl, naphthyl, fluorenyl, tetrahydronaphthyl, indanyl, idenyl, anthracenyl and the like.

The term “substituted aryl”, as used herein, refers to an aryl group, as previously defined, substituted by independent replacement of one, two, three or four of the hydrogen atoms thereon with substituents including, but not limited to unsubstituted or substituted C₁₋₄ alkyl (such as methyl or ethyl), unsubstituted or substituted C₃₋₆ cycloalkyl (such as cyclopropyl or cyclohexyl), halogen (such as fluoro, chloro or bromo), OH, NO₂, C₁₋₄ alkyloxy (such as methyloxy or ethyloxy), C₃₋₆ cycloalkyloxy (such as cyclopropyloxy or cyclohexyloxy), C₁₋₄ alkylamino (such as methylamino or ethylamino), C₁₋₄ dialkylamino (such as dimethylamino or diethylamino), C₃₋₆ cycloalkylamino (such as cyclopropylamino or cyclohexylamino).

As used herein, the term “3 to 14 membered heterocycle” means, unless otherwise stated, a stable 3, 4, 5, 6, 7, 8, 9, 10, 11, 12, 13, or 14-membered monocyclic, bicyclic or tricyclic ring (recognizing that rings with certain numbers of members cannot be bicyclic or tricyclic, e.g., a 3-membered ring can only be a monocyclic ring), any of which is saturated, unsaturated, or aromatic, and consists of carbon atoms and one or more ring heteroatoms, e.g., 1 or 1-2 or 1-3 or 1-4 heteroatoms, independently selected from the group consisting of nitrogen, oxygen, and sulfur, and including any bicyclic or tricyclic group in which any of the above-defined heterocyclic rings is fused to a second ring (e.g., a benzene ring). When a nitrogen atom is included in the ring it is either N or NH, depending on whether or not it is attached to a double bond in the ring (i.e., a hydrogen is present if needed to maintain the tri-valency of the nitrogen atom). The nitrogen atom may be substituted or unsubstituted (i.e., N or NR³ wherein R³ is H or C₁₋₄ alkyl as defined above). The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quaternized. Fused rings are also included (e.g. quinolinyl, iso quinolinyl, tetrahydroquinolinyl, 1H-purin-6-yl, phenothiazinyl, acridinyl or phenoxazinyl).

The term “substituted 3-14 membered heterocycle”, as used herein, refers to a 3-14 membered heterocycle group, as previously defined, substituted on 1-3 ring carbon atoms by independent replacement of one, two or three of the hydrogen atoms thereon with substituents including, but not limited to, unsubstituted or substituted C₁₋₄ alkyl (such as methyl or ethyl), unsubstituted or substituted C₃₋₆ cycloalkyl (such as cyclopropyl or cyclohexyl), halogen (such as fluoro, chloro or bromo), OH, NO₂, C₁₋₄ alkyloxy (such as methyloxy or ethyloxy), C₃₋₆ cycloalkyloxy (such as cyclopropyloxy or cyclohexyloxy), C₁₋₄ alkylamino (such as methylamino or ethylamino), C₁₋₄ dialkylamino (such as dimethylamino or diethylamino), C₃₋₆ cycloalkylamino (such as cyclopropylamino or cyclohexylamino).

As used herein, the term “aromatic heterocycle” or “heteroaryl” is intended to mean a stable 5, 6, 7, 8, 9, 10, 11, 12, 13 or 14-membered monocyclic or bicyclic aromatic ring (recognizing that rings with certain numbers of members cannot be a bicyclic aromatic, e.g., a 5-membered ring can only be a monocyclic aromatic ring), which consists of carbon atoms and one or more heteroatoms, e.g., 1 or 1-2 or 1-3 or 1-4 heteroatoms, independently selected from the group consisting of nitrogen, oxygen, and sulfur. In the case of bicyclic heterocyclic aromatic rings, only one of the two rings needs to be aromatic (e.g., 2,3-dihydroindole), though both may be (e.g., quinoline). The second ring can be fused as defined above for heterocycles. The nitrogen atom may be substituted or unsubstituted (i.e., N or NR³ wherein R³ is H or C₁₋₄ alkyl as defined above).

Examples of heterocycles include, but are not limited to, acridinyl, benzimidazolyl, benzofuranyl, 2,3-dihydrobenzofuranyl, benzothiofuranyl, benzothiophenyl, benzoxazolyl, benzo[1,3]dioxolyl, benzo[1,3]dioxanyl benzoxazolinyl, benzthiazolyl, benztriazolyl, benzisoxazolyl, benzisothiazolyl, benzo[1,2,5]thiadiazolyl, benzimidazolinyl, 3,4-dihydro-2H-benzo[b][1,4]dioxepinyl, 4,5,6,7-tetrahydro-benzo[b]thiophenyl, carbazolyl, 4aH-carbazolyl, cinnolinyl, decahydroquinolinyl, dihydrofuro[2,3-b]tetrahydrofuran, furanyl, furazanyl, imidazolidinyl, imidazolinyl, imidazolyl, 1H-indazolyl, indolenyl, indolinyl, indolyl, 3H-indolyl, isatinoyl, isobenzofuranyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

The term “lower alcohol”, as used herein, refers to a C₁₋₄alcohol, such as for example, methanol, ethanol, propanol, isopropanol, butanol, t-butanol, and the like.

The term “inert solvent”, as used herein, refers to a solvent that cannot react with the dissolved compounds including non-polar solvent such as hexane, toluene, diethyl ether, diisopropylether, chloroform, ethyl acetate, THF, dichloromethane; polar aprotic solvents such as acetonitrile, acetone, N,N-dimethylformamide, N,N-dimethylacetamide, dimethyl sulfoxide, pyridine, and polar protic solvents such as lower alcohol, acetic acid, formic acid and water.

Compounds of the Formula (Ia) include:
9a-{3-[(quinolin-2-yl-methyl)amino]propyl}-9-deoxo-9-dihydro-9a-aza-9a-homoerythromycin A;
9a-{3-[(quinolin-2-yl-methyl)amino]propyl}-3-O-decladinosyl-9-deoxo-9-dihydro-9a-aza-9a-homoerythromycin A;
9a-{3-[(quinolin-2-yl-methyl)amino]propyl}-3-O-decladinosyl-5-O-dedesosaminyl-9-deoxo-9-dihydro-9a-aza-9a-homoerythromycin A;
9a-{3-[(quinolin-3-yl-methyl)amino]propyl}-9-deoxo-9-dihydro-9a-aza-9a-homoerythromycin A;
9a-{3-[(quinolin-3-yl-methyl)amino]propyl}-3-O-decladinosyl-9-deoxo-9-dihydro-9a-aza-9a-homoerythromycin A;
9a-{3-[(quinolin-3-yl-methyl)amino]propyl}-3-O-decladinosyl-5-O-dedesosaminyl-9-deoxo-9-dihydro-9a-aza-9a-homoerythromycin A;
9a-[3-({[2-(ethyloxy)-naphthalen-1-yl]methyl}amino)propyl]-9-deoxo-9-dihydro-9a-aza-9a-homoerythromycin A;
9a-{3-[(naphtalen-1-yl-methyl)amino]propyl}-9-deoxo-9-dihydro-9a-aza-9a-homoerythromycin A;
9a-{3-[(naphtalen-1-yl-methyl)amino]propyl}-3-O-decladinosyl-9-deoxo-9-dihydro-9a-aza-9a-homoerythromycin A;
9a-{3-[(quinolin-4-yl-methyl)amino]propyl}-9-deoxo-9-dihydro-9a-aza-9a-homoerythromycin A;
9a-{3-[(quinolin-4-yl-methyl)amino]propyl}-3-O-decladinosyl-9-deoxo-9-dihydro-9a-aza-9a-homoerythromycin A;
9a-{2-[(naphtalen-1-yl-methyl)amino]ethyl}-9-deoxo-9-dihydro-9a-aza-9a-homoerythromycin A;
9a-{3-[methyl-(naphtalen-1-yl-methyl)amino]propyl}-9-deoxo-9-dihydro-9a-aza-9a-homoerythromycin A;
9a-{3-[(7-chloro-quinolin-4-yl)amino]propyl]}-9-deoxo-9-dihydro-9a-aza-9a-homoerythromycin A;
9a-{3-[(7-chloro-quinolin-4-yl)amino]propyl]}-3′N-demethyl-9-deoxo-9-dihydro-9a-aza-9a-homoerythromycin A;
9a-[3-(quinolin-4-yl-amino)propyl]-9-deoxo-9-dihydro-9a-aza-9a-homoerythromycin A;
9a-{3-[(7-chloro-quinolin-4-yl)amino]propyl]}-3-O-decladinosyl-9-deoxo-9-dihydro-9a-aza-9a-homoerythromycin A;
9a-[3-(quinolin-4-yl-amino)propyl]-3-O-decladinosyl-9-deoxo-9-dihydro-9a-aza-9a-homoerythromycin A;
9a-[3-(quinolin-4-yl-amino)propyl]-3′-N-demethyl-9-deoxo-9-dihydro-9a-aza-9a-homoerythromycin A;
9a-[3-(1H-purin-6-yl-amino)propyl]-9-deoxo-9-dihydro-9a-aza-9a-homoerythromycin A;
9a-{3-[(3-phenylpropanoyl)amino]propyl}-9a-aza-9-deoxo-9-dihydro-9a-homoerythromycin A formiate salt;
9a-{3-[(4-phenylbutanoyl)amino]propyl}-9a-aza-9-deoxo-9-dihydro-9a-homoerythromycin A formiate salt;
9a-{3-[(naphtalen-1-yl-acetyl)amino]propyl}-9a-aza-9-deoxo-9-dihydro-9a-homoerythromycin A formiate salt;
9a-{3-[(phenylacetyl)amino]propyl}-9a-aza-9-deoxo-9-dihydro-9a-homoerythromycin A formiate salt;
9a-{3-[(5-phenylpentanoyl)amino]propyl}-9a-aza-9-deoxo-9-dihydro-9a-homoerythromycin A formiate salt;
9a-{3-[(naphtalen-2-yl-acetyl)amino]propyl}-9a-aza-9-deoxo-9-dihydro-9a-homoerythromycin A formiate salt;
9a-(3-{[(4-methyl-1,3-oxazol-5-yl)carbonyl]amino}propyl)-9a-aza-9-deoxo-9-dihydro-9a-homoerythromycin A formiate salt;
9a-(3-{[(4-methyl-1,3-oxazol-5-yl)carbonyl]amino}propyl)-3-O-decladinosyl-5-O-dedesosaminyl-9a-aza-9-deoxo-9-dihydro-9a-homoerythromycin A formiate salt;
9a-{3-[(naphtalen-1-yl-acetyl)amino]propyl}-3-O-decladinosyl-9a-aza-9-deoxo-9-dihydro-9a-homoerythromycin A formiate salt;
9a-[3-({(2S)-2-[6-(methyloxy)-naphthalen-2-yl]propanoyl}amino)propyl]-3-O-decladinosyl-9a-aza-9-deoxo-9-dihydro-9a-homoerythromycin A formiate salt;
9a-{1-[(phenylmethyl)amino]propanoyl}-9-deoxo-9-dihydro-9a-aza-9a-homoerythromycin A formiate salt;
9a-{1-[(2-phenylethyl)amino]propanoyl}-9-deoxo-9-dihydro-9a-aza-9a-homoerythromycin A formiate salt;
9a-{1-[(3-phenylpropyl)amino]propanoyl}-9-deoxo-9-dihydro-9a-aza-9a-homoerythromycin A formiate salt;
9a-{1-[(4-phenylbutyl)amino]propanoyl}-9-deoxo-9-dihydro-9a-aza-9a-homoerythromycin A formiate salt;
9a-{1-[(1S)-1-(1-naphthalenyl)ethyl]amino)propanoyl}-9-deoxo-9-dihydro-9a-aza-9a-homoerythromycin A formiate salt;
9a-{1-(2-naphthalenylmethyl)amino]propanoyl}-9-deoxo-9-dihydro-9a-aza-9a-homoerythromycin A formiate salt;
9a-{1-[(1S)-1-(1-naphthalenyl)ethyl]amino)propanoyl}-9-deoxo-9-dihydro-9a-aza-9a-homoerythromycin A diacetate salt;
9a-{1-[(1S)-1-(1-naphthalenyl)ethyl]amino)propanoyl}-9-deoxo-9-dihydro-9a-aza-9a-homoerythromycin A;
9a-[3-(quinolin-4-yl-amino)propyl]-9-deoxo-9-dihydro-9a-aza-9a-homoerythromycin A triacetate salt;
9a-{3-[(3-phenylpropanoyl)amino]propyl}-9a-aza-9-deoxo-9-dihydro-9a-homoerythromycin A;
9a-{3-[(3-phenylpropanoyl)amino]propyl}-9a-aza-9-deoxo-9-dihydro-9a-homoerythromycin A diacetate salt;
9a-{1-[(3-phenylpropyl)amino]propanoyl}-9-deoxo-9-dihydro-9a-aza-9a-homoerythromycin A;
9a-{1-[(3-phenylpropyl)amino]propanoyl}-9-deoxo-9-dihydro-9a-aza-9a-homoerythromycin A diacetate salt;
9a-{3-[(naphtalen-2-yl-acetyl)amino]propyl}-9a-aza-9-deoxo-9-dihydro-9a-homoerythromycin A;
9a-{3-[(naphtalen-2-yl-acetyl)amino]propyl}-9a-aza-9-deoxo-9-dihydro-9a-homoerythromycin A diacetate salt;
9a-{3-[(quinolin-4-yl-methyl)amino]propyl}-3-O-decladinosyl-9-deoxo-9-dihydro-9a-aza-9a-homoerythromycin A diacetate salt;
9a-{3-[(naphtalen-2-yl-methyl)amino]propyl}-9-deoxo-9-dihydro-9a-aza-9a-homoerythromycin A;
9a-{3-[(1,2,3,4-tetrahydro-quinolin-4-yl-methyl)amino]propyl}-9-deoxo-9-dihydro-9a-aza-9a-homoerythromycin A;
9a-{3-[(7-chloro-quinolin-4-yl)amino]propyl]}-3-O-decladinosyl-5-O-dedesosaminyl-9-deoxo-9-dihydro-9a-aza-9a-homoerythromycin A;
9a-(3-{[3-(quinolin-4-yl)propanoyl]amino}propyl)-9-deoxo-9-dihydro-9a-aza-9a-homoerythromycin A;
9a-[3-({[4-(methyloxy)phenyl]acetyl}amino)propyl]-9-deoxo-9-dihydro-9a-aza-9a-homoerythromycin A formiate salt;
9a-[3-({[2,4,5-trifluoro-3-(methyloxy)phenyl]carbonyl}amino)propyl]-9-deoxo-9-dihydro-9a-aza-9a-homoerythromycin A formiate salt;
9a-{3-[(N-acetyl-4-fluorophenylalanyl)amino]propyl}-9-deoxo-9-dihydro-9a-aza-9a-homoerythromycin A formiate salt;
9a-(3-{[(3-nitrophenyl)acetyl]amino}propyl)-9-deoxo-9-dihydro-9a-aza-9a-homoerythromycin A formiate salt;
9a-(3-{[(3-chlorophenyl)acetyl]amino}propyl)-9-deoxo-9-dihydro-9a-aza-9a-homoerythromycin A formiate salt;
9a-(3-{[(4-chlorophenyl)acetyl]amino}propyl)-9-deoxo-9-dihydro-9a-aza-9a-homoerythromycin A formiate salt;
9a-(3-{[(4-nitrophenyl)acetyl]amino}propyl)-9-deoxo-9-dihydro-9a-aza-9a-homoerythromycin A formiate salt;
9a-{3-[(4-oxo-4-phenylbutanoyl)amino]propyl}-9-deoxo-9-dihydro-9a-aza-9a-homoerythromycin A formiate salt;
9a-(3-{[(5-chloro-2-nitrophenyl)carbonyl]amino}propyl)-9-deoxo-9-dihydro-9a-aza-9a-homoerythromycin A formiate salt;
9a-(3-{[(2-nitrophenyl)acetyl]amino}propyl)-9-deoxo-9-dihydro-9a-aza-9a-homoerythromycin A formiate salt;
9a-(3-{[(4-fluorophenyl)acetyl]amino}propyl)-9-deoxo-9-dihydro-9a-aza-9a-homoerythromycin A formiate salt;
9a-(3-{[(2-fluorophenyl)acetyl]amino}propyl)-9-deoxo-9-dihydro-9a-aza-9a-homoerythromycin A formiate salt;
9a-(3-{[(4-fluorophenyl)propanoyl]amino}propyl)-9-deoxo-9-dihydro-9a-aza-9a-homoerythromycin A formiate salt;
9a-{3-[(2-phenylpropanoyl)amino]propyl}-9-deoxo-9-dihydro-9a-aza-9a-homoerythromycin A formiate salt;
9ₐ-(3-{[(2,4-dichlorophenyl)carbonyl]amino}propyl)-9-deoxo-9-dihydro-9a-aza-9a-homoerythromycin A formiate salt;
9a-(3-{[3-(3-nitrophenyl)-2-propenoyl]amino}propyl)-9-deoxo-9-dihydro-9a-aza-9a-homoerythromycin A formiate salt;
9a-(3-{[4-(4-nitrophenyl)butanoyl]amino}propyl)-9-deoxo-9-dihydro-9a-aza-9a-homoerythromycin A formiate salt;
9a-(3-{[(4-bromophenyl)carbonyl]amino}propyl)-9-deoxo-9-dihydro-9a-aza-9a-homoerythromycin A formiate salt;
9a-(3-{[(2E)-3-(quinolin-3-yl)-2-propenoyl]amino}propyl)-9-deoxo-9-dihydro-9a-aza-9a-homoerythromycin A;
9a-{3-[(7-chloro-quinolin-4-yl)amino]propyl]}-9-deoxo-9-dihydro-9a-aza-9a-homoerythromycin A triacetate salt;
9a-{3-[(quinolin-4-yl-methyl)amino]propyl}-9-deoxo-9-dihydro-9a-aza-9a-homoerythromycin A diacetate salt;
9a-{3-[(7-chloro-quinolin-4-yl)amino]propyl]}-3-O-decladinosyl-9-deoxo-9-dihydro-9a-aza-9a-homoerythromycin A triacetate salt;
   and/or pharmaceutically acceptable derivatives thereof.

Compounds of Formula (I) include:
9a-{3-[(pyridine-4-yl-methyl)amino]propyl}-9-deoxo-9-dihydro-9a-aza-9a-homoerythromycin A;
9a-{3-[(pyridine-4-yl-methyl)amino]propyl}-3-O-decladinosyl-9-deoxo-9-dihydro-9a-aza-9a-homoerythromycin A;
9a-{3-[(pyridine-3-yl-methyl)amino]propyl}-9-deoxo-9-dihydro-9a-aza-9a-homoerythromycin A;
9a-{3-[(pyridine-3-yl-methyl)amino]propyl}-3-O-decladinosyl-9-deoxo-9-dihydro-9a-aza-9a-homoerythromycin A;
9a-{3-[(pyridin-2-yl-methyl)amino]propyl}-9-deoxo-9-dihydro-9a-aza-9a-homoerythromycin A;
9a-{3-[(3-phenylpropyl)amino]propyl}-9-deoxo-9-dihydro-9a-aza-9a-homoerythromycin A;
9a-{3-[(2-phenylethyl)amino]propyl}-9-deoxo-9-dihydro-9a-aza-9a-homoerythromycin A;
   and/or pharmaceutically acceptable derivatives thereof.

“Treating” or “treatment” of malaria includes
i. preventing or delaying the appearance of clinical symptoms of malaria developing in a mammal that has been in contact with the parasite.
ii. inhibiting the malaria, i.e., arresting, reducing or delaying the development of malaria or a relapse thereof or at least one clinical or subclinical symptom thereof, or
iii. relieving or attenuating one or more of the clinical or subclinical symptoms of malaria.

The benefit to a subject to be treated is either statistically significant or at least perceptible to the patient or to the physician.

“Prophylactic treatment” of malaria includes treating subjects who are at risk of developing malaria. This includes the treatment of subjects who have been exposed to malaria-bearing mosquitoes, the treatment of subjects who intend to travel to a country where malaria is endemic and the treatment of subjects who otherwise risk exposure to malaria-bearing mosquitoes.

“Maintenance therapy” is preventive therapy that follows successful initial treatment of the acute phase of the illness where regular (usually smaller) doses of the drug are delivered to the patient to prevent recurrence and worsening of the disease. The Plasmodium vivax and P. ovale parasites have dormant liver stages that can remain silent for years. Maintenance therapy for these strains is particularly important. The hallmarks of the acute phase include symptoms like chills and fever.

“Subject” refers to an animal, in particular a mammal and more particularly to a human or a domestic animal or an animal serving as a model for a disease (e.g., mouse, monkey, etc.). In one aspect, the subject is a human. As used herein, the term patient is used synonymously with subject.

A “therapeutically effective amount” means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated and will be ultimately at the discretion of the attendant physician.

### Pharmaceutical Compositions

While it is possible that, for use in the methods of the invention, a compound of formula I may be administered as the bulk substance, it is preferable to present the active ingredient in a pharmaceutical formulation, for example, wherein the agent is in admixture with a pharmaceutically acceptable carrier selected with regard to the intended route of administration and standard pharmaceutical practice.

The term “carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition. The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

It will be appreciated that pharmaceutical compositions for use in accordance with the present invention may be in the form of oral, parenteral, transdermal, inhalation, sublingual, topical, implant, nasal, or enterally administered (or other mucosally administered) suspensions, capsules or tablets, which may be formulated in conventional manner using one or more pharmaceutically acceptable carriers or excipients.

There may be different composition/formulation requirements depending on the different delivery systems. It is to be understood that not all of the compounds need to be administered by the same route. Likewise, if the composition comprises more than one active component, then those components may be administered by the same or different routes. By way of example, the pharmaceutical composition of the present invention may be formulated to be delivered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be delivered by multiple routes.

The present invention further relates to pharmaceutical formulations containing a therapeutically effective quantity of a compound of formula I or one of its salts mixed with a pharmaceutically acceptable vehicle. The pharmaceutical formulations of the present invention can be liquids that are suitable for oral, mucosal and/or parenteral administration, for example, drops, syrups, solutions, injectable solutions that are ready for use or are prepared by the dilution of a freeze-dried product but are preferably solid or semisolid as tablets, capsules, granules, powders, pellets, pessaries, suppositories, creams, salves, gels, ointments; or solutions, suspensions, emulsions, or other forms suitable for administration by the transdermal route or by inhalation.

The compounds of the invention can be administered for immediate-, delayed-, modified-, sustained-, pulsed- or controlled-release applications.

In one aspect, oral compositions are slow, delayed or positioned release (e.g., enteric especially colonic release) tablets or capsules. This release profile can be achieved without limitation by use of a coating resistant to conditions within the stomach but releasing the contents in the colon or other portion of the GI tract wherein a lesion or inflammation site has been identified. Or a delayed release can be achieved by a coating that is simply slow to disintegrate. Or the two (delayed and positioned release) profiles can be combined in a single formulation by choice of one or more appropriate coatings and other excipients. Such formulations constitute a further feature of the present invention.

Suitable compositions for delayed or positioned release and/or enteric coated oral formulations include tablet formulations film coated with materials that are water resistant, pH sensitive, digested or emulsified by intestinal juices or sloughed off at a slow but regular rate when moistened. Suitable coating materials include, but are not limited to, hydroxypropyl methylcellulose, ethyl cellulose, cellulose acetate phthalate, polyvinyl acetate phthalate, hydroxypropyl methylcellulose phthalate, polymers of metacrylic acid and its esters, and combinations thereof. Plasticizers such as, but not limited to polyethylene glycol, dibutylphthalate, triacetin and castor oil may be used. A pigment may also be used to color the film. Suppositories are be prepared by using carriers like cocoa butter, suppository bases such as Suppocire C, and Suppocire NA50 (supplied by Gattefossé Deutschland GmbH, D-Weil am Rhein, Germany) and other Suppocire type excipients obtained by interesterification of hydrogenated palm oil and palm kernel oil (C8-C18 triglycerides), esterification of glycerol and specific fatty acids, or polyglycosylated glycerides, and whitepsol (hydrogenated plant oils derivatives with additives). Enemas are formulated by using the appropriate active compound according to the present invention and solvents or excipients for suspensions. Suspensions are produced by using micronized compounds, and appropriate vehicle containing suspension stabilizing agents, thickeners and emulsifiers like carboxymethylcellulose and salts thereof, polyacrylic acid and salts thereof, carboxyvinyl polymers and salts thereof, alginic acid and salts thereof, propylene glycol alginate, chitosan, hydroxypropylcellulose, hydroxypropylmethylcellulose, hydroxyethylcellulose, ethylcellulose, methylcellulose, polyvinyl alcohol, polyvinyl pyrrolidone, N-vinylacetamide polymer, polyvinyl methacrylate, polyethylene glycol, pluronic, gelatin, methyl vinyl ether-maleic anhydride copolymer, soluble starch, pullulan and a copolymer of methyl acrylate and 2-ethylhexyl acrylate lecithin, lecithin derivatives, propylene glycol fatty acid esters, glycerin fatty acid esters, sorbitan fatty acid esters, polyoxyethylene sorbitan fatty acid esters, polyethylene glycol fatty acid esters, polyoxyethylene hydrated caster oil, polyoxyethylene alkyl ethers, and pluronic and appropriate buffer system in pH range of 6.5 to 8. The use of preservatives, masking agents is suitable. The average diameter of micronized particles can be between 1 and 20 micrometers, or can be less than 1 micrometer. Compounds can also be incorporated in the formulation by using their water-soluble salt forms.

Alternatively, materials may be incorporated into the matrix of the tablet e.g. hydroxypropyl methylcellulose, ethyl cellulose or polymers of acrylic and metacrylic acid esters. These latter materials may also be applied to tablets by compression coating.

Pharmaceutical compositions can be prepared by mixing a therapeutically effective amount of the active substance with a pharmaceutically acceptable carrier that can have different forms, depending on the way of administration. Pharmaceutical compositions can be prepared by using conventional pharmaceutical excipients and methods of preparation. The forms for oral administration can be capsules, powders or tablets where usual solid vehicles including lactose, starch, glucose, methylcellulose, magnesium stearate, di-calcium phosphate, mannitol may be added, as well as usual liquid oral excipients including, but not limited to, ethanol, glycerol, and water. All excipients may be mixed with disintegrating agents, solvents, granulating agents, moisturizers and binders. When a solid carrier is used for preparation of oral compositions (e.g., starch, sugar, kaolin, binders disintegrating agents) preparation can be in the form of powder, capsules containing granules or coated particles, tablets, hard gelatin capsules, or granules without limitation, and the amount of the solid carrier can vary (between 1 mg to 1 g). Tablets and capsules are the preferred oral composition forms.

Pharmaceutical compositions containing compounds of the present invention may be in any form suitable for the intended method of administration, including, for example, a solution, a suspension, or an emulsion. Liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soybean oil, coconut oil, olive oil, safflower oil, cottonseed oil, and the like. For parenteral administration, the carrier can also be an oily ester such as ethyl oleate, isopropyl myristate, and the like. Compositions of the present invention may also be in the form of microparticles, microcapsules, liposomal encapsulates, and the like, as well as combinations of any two or more thereof.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral compositions include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable odorants for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants include, but are not limited to, sodium lauryl sulfate and polysorbates.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetriacetic acid (EDTA), thiourea, tocopherol and butyl hydroxyanisole.

The compounds of the invention may also, for example, be formulated as suppositories e.g., containing conventional suppository bases for use in human or veterinary medicine or as pessaries e.g., containing conventional pessary bases.

The compounds according to the invention may be formulated for topical administration, for use in human and veterinary medicine, in the form of ointments, creams, gels, hydrogels, lotions, solutions, shampoos, powders (including spray or dusting powders), pessaries, tampons, sprays, dips, aerosols, drops (e.g., eye ear or nose drops) or pour-ons.

For application topically to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax, sorbitan monostearate, a polyethylene glycol, liquid paraffin, polysorbate 60, cetyl esters wax, cetearyl alcohol, 2-octyldodecanol, benzyl alcohol, and water. Such compositions may also contain other pharmaceutically acceptable excipients, such as polymers, oils, liquid carriers, surfactants, buffers, preservatives, stabilizers, antioxidants, moisturizers, emollients, colorants, and odorants.

Examples of pharmaceutically acceptable polymers suitable for such topical compositions include, but are not limited to, acrylic polymers; cellulose derivatives, such as carboxymethylcellulose sodium, methylcellulose or hydroxypropylcellulose; natural polymers, such as alginates, tragacanth, pectin, xanthan and cytosan.

As indicated, the compound of the present invention can be administered intranasally or by inhalation and is conveniently delivered in the form of a dry powder inhaler or an aerosol spray presentation from a pressurized container, pump, spray or nebulizer with the use of a suitable propellant, e.g., a hydrofluoroalkane such as 1,1,1,2-tetrafluoroethane (HFA 134AT) or 1,1,1,2,3,3,3-heptafluoropropane (HFA 227EA), or a mixture thereof. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant, e.g., sorbitan trioleate.

Capsules and cartridges (made, for example, from gelatin) for use in an inhaler or insufflator may be formulated to contain a powder mix of the compound and a suitable powder base such as lactose or starch.

For topical administration by inhalation the compounds according to the invention may be delivered for use in human or veterinary medicine via a nebulizer.

The pharmaceutical compositions of the invention may contain from 0.01 to 99% weight per volume of the active material. For topical administration, for example, the composition will generally contain from 0.01-10%, more preferably 0.01-1% of the active material.

A therapeutically effective amount of the compound of the present invention can be determined by methods known in the art. The therapeutically effective quantities will depend on the age and on the general physiological condition of the patient, the route of administration and the pharmaceutical formulation used. It will also be determine by the strain of malaria parasite that has infected the subject. The therapeutic doses will generally be between about 10 and 2000 mg/day and preferably between about 30 and 1500 mg/day. Other ranges may be used, including, for example, 50-500 mg/day, 50-300 mg/day, 100-200 mg/day. The amount of the compound required for prophylactic treatment, referred to as a prophylactically-effective dosage, is generally the same as described for therapeutic treatment.

Administration may be once a day, twice a day, or more often, and may be decreased during a maintenance phase of the disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art.

### Method of Preparation:

Compounds of Formula (I) and pharmaceutically acceptable derivatives thereof may be prepared by the general methods outlined hereinafter, said methods constituting a further aspect of the invention. In the following description, the groups R¹, R², R³, R⁴, X, A, Q and m have the meaning defined for the compounds of Formula (I) unless otherwise stated.

It will be appreciated by those skilled in the art that it may be desirable to use protected derivatives of intermediates used in the preparation of the compounds of Formula (I). Protection and deprotection of functional groups may be performed by methods known in the art. Hydroxyl or amino groups may be protected with any hydroxyl or amino protecting group (for example, as described in Green and Wuts. Protective Groups in Organic Synthesis. John Wiley and Sons, New York, 1999). The protecting groups may be removed by conventional techniques. For example, acyl groups (such as alkanoyl, alkoxycarbonyl and aryloyl groups) may be removed by solvolysis (e.g., by hydrolysis under acidic or basic conditions). Arylmethoxycarbonyl groups (e.g., benzyloxycarbonyl) may be cleaved by hydrogenolysis in the presence of a catalyst such as palladium-on-carbon.

The synthesis of the target compound is completed by removing any protecting groups, which are present in the penultimate intermediate using standard techniques, which are well-known to those skilled in the art. The final product is then purified, as necessary, using standard techniques such as silica gel chromatography, HPLC on silica gel, and the like or by recrystallization.

The compounds of Formula (I) wherein X is NR³ and R³ is hydrogen, and Q is C₁₋₄alkylene as described herein and pharmaceutically acceptable derivatives thereof can be prepared from the compound of Formula (IV)

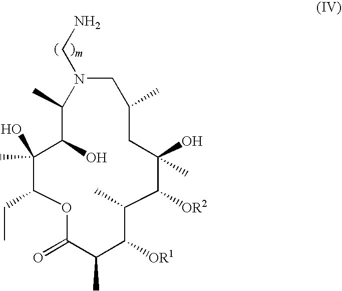

by reductive alkylation with aldehyde of formula (V),

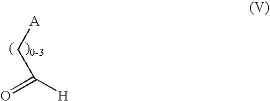

using reducing agent in lower alcohol (such as methanol), dichlohomethane, or some other inert solvent, at a temperature from about 0° to about reflux temperature of the solvent. Suitable reducing agents are for example metalborohydrides (such as sodium borohydride) or hydrogen in the presence of catalyst such as palladium on carbon or platinum on carbon.

The compounds of Formula (I), wherein X is NR³ and R³ is C₁₋₄ alkyl as described herein and pharmaceutically acceptable derivatives thereof can be prepared from the compound of Formula (I), wherein X is NR³ and R³ is hydrogen by reductive alkylation with aldehyde of formula (VIa) or (VIb) using suitable reducing agents in a solvent such as methanol, halohydrocarbones (e.g. dichloromethane or chloroform) or in DMF. Suitable reducing agents are for example metalborohydrides (such as sodium borohydride) or hydrogen in the presence of catalyst such as palladium on carbon or platinum on carbon.

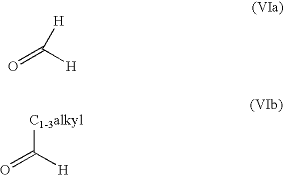

In a further embodiment of the invention the compounds of Formula (I), wherein Q is a bond and X is NR³ may be prepared by reaction of compound of Formula (IV) with compound of formula A-L (VII), wherein L is suitable leaving group. Suitable leaving groups for this reaction include halogen (e.g. chlorine, bromine or iodine). The reaction is suitably carried out in a solvent such as halohydrocarbon (e.g. dichloromethane), an ether (e.g. tetrahydrofuran, dimethoxyethane), acetonitrile or ethyl acetate and the like, dimethylsulphoxide, N,N-dimethylformamide, 1-methyl-pyrrolidone and in the presence of base. Examples of the bases which may be used include organic base such as diisopropylethylamine, triethylamine, 1,8-diazabicyclo[5.4.0.]undec-7-ene (DBU), or inorganic base such as potassium hydroxide, ammonium hydroxide, sodium hydride, sodium hydroxide, potassium hydride and the like. The reaction is preferably carried out at the temperature from 0° C. to 120° C.

In another embodiment the compound of Formula (I), wherein X is NHC(O) may be prepared starting from compound of Formula (IV) with suitable activated derivative of carboxylic acid of Formula HOC(O)(CH₂)₁₋₄A (VIII). Suitable activated derivatives of the carboxyl group include the corresponding acyl halide, mixed anhydride or activated ester such as a thioester. The reaction is suitably carried out in a suitable aprotic solvent such as a halohydrocarbon (e.g. dichloromethane) or N,N-dimethylformamide optionally in the presence of a tertiary organic base such as dimethylaminopyridine or triethylamine or in the presence of inorganic base (eg. sodium hydroxide) and at a temperature within the range of 0° to 120° C.

In a further embodiment reaction between compound of Formula (IV) and compound of Formula (VIII) may be carried out in the in the presence of carbodiimides such as dicyclohexylcarbodiimide (DCC), 1,8-diazabicyclo[5.4.0.]undec-7-ene (DBU) or 1-(3-dimethylaminopropyl)-3-ethylcarbodiimide (EDC).

In yet another embodiment the compound of Formula (I), wherein X is C(O)NH may be prepared starting from compound of Formula (IX) with amine of Formula A-(CH₂)₁₋₄—NH₂ (X). The reaction is suitably carried out in a suitable inert solvent such as a halohydrocarbon (e.g. dichloromethane) or N,N-dimethylformamide, lower alcohol (e.g. tert-butanol, iso-propanol, ethanol or methanol) optionally in the presence of EDC or a organic base such as dimethylaminopyridine, triethylamine or DBU, in the presence of inorganic base (eg sodium hydroxide, lithium hydroxide and potassium hydroxide) and at a temperature within the range of 0° to 120° C.

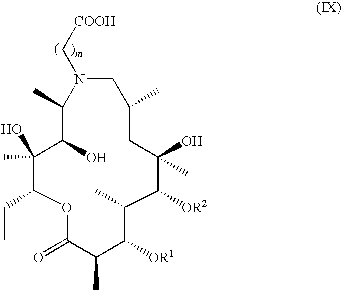

The compound of Formula (IV) may be prepared by reaction of 9-deoxo-9-dihydro-9a-aza-9a-homoerythromycin A (J. Chem. Soc. Perkin Trans. I (1986) pages 1881-1890) with nitrile-containing electrophiles of Formula (XI), wherein L is suitable leaving group followed by reduction of nitrile to amino group. Suitable leaving groups for this reaction include halogen (e.g. chlorine, bromine or iodine), OTs or OMs group.

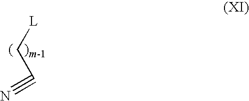

The compound of Formula (IX) may be prepared by reaction of 9-deoxo-9-dihydro-9a-aza-9a-homoerythromycin A (J. Chem. Soc. Perkin Trans. I (1986) pages 1881-1890) with compound of Formula (XII), wherein L is suitable leaving group followed by ester hydrolysis under basic conditions. Suitable leaving groups for this reaction include halogen (e.g. chlorine, bromine or iodine)

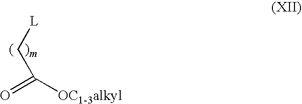

In a particular embodiment the compound of Formula (IX), wherein m is 2 may be prepared by reaction of 9-deoxo-9-dihydro-9a-aza-9a-homoerythromycin A with methyl or ethyl acrylate followed by ester hydrolysis under basic conditions.

Compounds of Formula (V), (VIa), (VIb), (VII), (VIII), (X), (XI), (XII) and 9-deoxo-9-dihydro-9a-aza-9a-homoerythromycin A are commercially available or may be readily prepared by methods well known in the art.

Pharmaceutically acceptable acid addition salts, which also represent an object of the present invention, were obtained by reaction compound of Formula (I) with an at least equimolar amount of the corresponding inorganic or organic acid such as hydrochloric acid, hydroiodic acid, sulfuric acid, phosphoric acid, acetic acid, trifluoroacetic acid, propionic acid, benzoic acid, benzenesulfonic acid, methane sulfonic acid, laurylsulfonic acid, stearic acid, palmitic acid, succinic acid, ethylsuccinic acid, lactobionic acid, oxalic acid, salicylic acid and similar acid, in a solvent inert to the reaction. Addition salts are isolated by evaporating the solvent or, alternatively, by filtration after a spontaneous precipitation or a precipitation by the addition of a non-polar cosolvent.

Compounds of the Formula (I) and pharmaceutically acceptable addition salts with inorganic or organic acids thereof possess an antimalarial activity in vitro.

### Biological Assays

The potential for the compounds of the present invention to have a therapeutic benefit in the treatment and/or prophylaxis of malaria may be demonstrated, for example, using one or more of the following assays.

### In Vitro Screening Protocol I

The in vitro screens for intrinsic antimalarial activity were based on modifications of the procedures described by Desjardins R E, Canfield C J, Haynes J D, Chulay J D. (Quantitative assessment of antimalarial activity in vitro by a semiautomated microdilution technique. Antimicrob Agents Chemother. 16 (6) (1979) 710-718), Chulay J D, Haynes J D, Diggs C L. (Plasmodium falciparum: Assessment of in vitro growth by [3H]hypoxanthine incorporation Exp. Parasitol. 55 (1983) 138-146.), and Milhous W K, Weatherly N F, Bowdre J H, Desjardins R E. (In vitro activities of and mechanisms of resistance to antifol antimalarial drugs Antimicrob. Agents Chemother. 27 (4) (1985) 525-530). The system was limited to the assessment of the intrinsic activity against the erythrocytic asexual life cycle (blood schizontocides). Two Plasmodium falciparum clones from CDC/Indochina III (W-2) and CDC/Sierra Leone I (D-6) (Oduola A M, Weatherly N F, Bowdre J H, Desjardins R E. Plasmodium falciparum: cloning by single-erythrocyte micromanipulation and heterogeneity in vitro. Exp Parasitol. 66 (1) (1988) 86-95) were used for all assays. TM91C235, a multiple drug resistant isolate from Thailand (Antimicrob. Agents Chemother. 46 (8) (2002) 2627 and Antimicrob. Agents Chemother. 43 (3) (1999) 598-602), was used for the prescreening assay. W-2 is resistant to chloroquine, quinine, and pyrimethamine and susceptible to mefloquine. D-6 tends to be more resistant to mefloquine and susceptible to chloroquine, quinine, and pyrimethamine. All documents cited in this paragraph are incorporated by reference in their entirety.

All parasites were maintained in continuous long term cultures in RPMI-1640 medium supplemented with 6% washed human A positive (A+)(erythrocytes, 25 mM Hepes, 32 nM NaHCO₃, and 10% heat inactivated A+ human plasma or ALBUMAX® (lipid-rich bovine serum albumin; Invitrogen, Carlsbad, Calif.). All cultures and assays were conducted at 37° C. under an atmosphere of 5% CO₂ and 5% O₂, with a balance of N₂.

### PreScreening Assay

The prescreening assay uses TM91C235 diluted at a 0.4% parasitemia in a 1% hematocrit in folic acid free and p-aminobenzoic acid free media RPMI-1640 and ALBUMAX®. One mg of the compound is typically dissolved in 100 μl of dimethyl sulfoxide (DMSO). The compound is further diluted in folate free (FF) culture medium with ALBUMAX® for the first initial starting concentration. The rest of the stock drug solution was kept at −70° C. The isolate was preexposed, in duplicate, at three concentrations (25,000 ng/ml, 2,500 ng/ml, and 25 ng/ml) of the test compound for 48 hr in a 96-well microtiter plate (MTP) using the BIOMEK® 2000 automated laboratory workstation. Each MTP contains chloroquine as control to assess the relative activity of the compound and to monitor the response of TM91C235.

After the preincubation, [³H]-hypoxanthine was added to each well of the MTP. (The assay relies on the incorporation of radiolabeled hypoxanthine by the parasites, which indicates reproduction, and inhibition of isotope incorporation was attributed to activity of known or candidate antimalarial drugs). After 72 hours of total incubation time, the MTP were frozen to lyse the erythrocytes and parasites. The parasite DNA was recovered by harvesting the lysate onto glass-fiber filter plates using a Packard FilterMate™ Cell Harvester. The radioactivity was counted on a Packard TopCount™ microplate scintillation counter. The results were recorded as counts per minutes (CPM) per well at each drug concentration divided by the arithmetic mean of the CPM from the three untreated infection parasite control wells.

### Serial Dilution Assay

If a compound did not affect parasite growth at 25,000 ng/ml, it was classified as inactive. If a compound suppressed greater than two standard deviations from the arithmetic mean of the untreated infection controls at 25,000 ng/ml, but less than 50% at 2,500 ng/ml, the compound was designated as partially active. However, if a compound suppressed greater than 50% of the incorporation of [³H]-hypoxanthine relative to untreated infection control parasites at 2,500 ng/ml, the compound was classified as fully active and was further evaluated by two-fold serial dilutions to determine the IC₅₀ value (50% inhibitory concentration).

The serial dilution assay was conducted using the same assay conditions and stock solution of the compound used for the preliminary screen, described above. Both the D-6 and the W-2 clones were used. The compounds were diluted two-fold over 11 different concentration ranges with a starting concentration that was based on the preliminary screen. For each drug, the concentration response profile was determined and 50% inhibitory concentrations (IC₅₀) were determined by using a non-linear, logistic dose response analysis program. If the results from this assay did not agree with the concentration ranges of the preliminary screen, the assay was repeated. For each assay, the IC₅₀ for each clone was determined against the known antimalarials chloroquine and mefloquine. These control values established the compound's relative parasite susceptibility profile compared to known antimalarials.

IC₅₀s can be similarly determined for drug-resistant isolates/clones from a wide variety of geographic locations by using different isolates/clones in the assays described herein. The assays described above can be repeated using both samples according to Formula I and isolates/clones from different malaria strains to determine the antilmalarial activity of the compounds. For Example, the above assays can be used to determine the IC₅₀ values for malarial strains TM91C235 (reported to be resistant to mefloquine, chloroquine, and quinine), D6 (reported to be resistant to mefloquine), and W2 (reported to be resistant to chloroquine).

### In Vitro Screening Protocol II

The protocol described here is a modification of the in vitro screening protocol I and the details of the methodology are given below.

### I. Materials

### Parasite

Plasmodium falciparum strains 3D7A and W2.

### Culture Medium.

The culture medium comprised RPMI 1640 with 25 mM HEPES, sodium bicarbonate and glutamine (GIBCO™ cat. ref.: 52400), supplemented with 10% of pooled human sera AB (Bioreclamation HMSRM-AB)) and HT supplement (0.15 mM hypoxanthine and 24 μM thymidine), (GIBCO™ cat. ref.: 41065). Human sera were decomplemented 30 min. at 56° C., aliquoted and stored frozen at −20° C. until use in this culture medium.

This culture medium (“complete medium”) was usually prepared fresh just before use and pre-warmed to 37° C.

### Red Blood Cells

Red blood cells AB− stock suspensions were prepared from whole blood bags coming from incomplete blood donation, provided by the Spanish Red Cross (<25 days after sampling). This “whole blood” was aliquoted and stored at 4° C.

To prepare red blood cells for the assay, the whole blood was centrifuged and washed 3 times with RPMI without serum. The upper phase, containing white blood cells was removed. The washed red blood cells were kept as a 50% suspension in complete medium. The prepared cells were stored at 4° C. and were employed in the assay at any time up to 4 days after preparation.

### II. Compounds

### Compound Preparation

Test compounds were dissolved at 2 mg/ml in 100% DMSO on the day of the assay. If necessary, complete dissolution was achieved by gentle heating (the mixture was heated at a temperature <37° C.) and sonication (sonication bath).

Before test compounds were added to the parasites, the percentage of DMSO in the compound solution was reduced by further dilutions of the solution with culture medium prepared in the same way as described above for complete medium, but which did not contain hypoxanthine. The final concentration of DMSO in the assay plates was not permitted to exceed 0.2%, so that it did not produce any detectable undesired effects on the development of the parasite.

For IC₅₀ determinations, 10 serial 2-fold dilutions were prepared in complete medium in the presence of a constant amount of DMSO. Any obvious signs of insolubility of the stock solutions in 100% DMSO or precipitation when these solutions were diluted in assay media, were recorded.

III. Plasmodium falciparum Culture (Parasite)

Plasmodium falciparum strains were maintained in complete medium at 5% of hematocrit in continuous culture using a method adapted from Trager W. and Jensen J. B. (Human malaria parasites in continuous culture Science 193 (4254) (1976) 673-675) and Trager W. (Cultivation of malaria parasites Methods Cell Biol. 45 (1994) 7-26).

The parasitemia was calculated by counting the percentage of parasitized erythrocytes by optical microscopy. Thin films of blood were made every day from each culture flask, fixed with methanol and stained for 10 min. in Giemsa (Merck, cat ref.: 1.09204) at 10% in buffered water pH 7.2. The glass slides were observed and counted with an optical microscope (Nikon, Eclipse E200) equipped with a 100× immersion oil objective.

The culture was maintained at 5% of hematocrit, with a daily change of medium and was diluted when parasitemia had reached about 5%. The parasite population was asynchronous and showed a regular rate of growth of 3 to 3.5 times the initial number of parasites daily.

Growth was achieved in culture flasks (canted neck, Corning) incubated at 37° C. under low oxygen atmosphere (5% CO₂, 5% O₂, 95% N₂).

### IV. IC₅₀ Assay

[³H] Hypoxanthine incorporation assay was conducted using a method adapted from Desjardins R. E. et al. (Quantitative assessment of antimalarial activity in vitro by a semiautomated microdilution technique. Antimicrob. Agents Chemother., 16 (6) (1979) 710-718). The assays were performed in 96 wells flat bottom microplates.

1. Serial dilutions of each test compound (25 μl of a 5× solution/well) were deposited in one row of assay microtiter plate. Compounds of this invention were tested in this assay. Chloroquine and Azithromycin were used as control compounds for each assay.

2. The inoculum was prepared as a suspension of parasitized red blood cells (PRBCs) at 1.5% of hematocrit and 0.4% of parasitemia in culture medium prepared in the same way as described above for complete medium, but which did not contain hypoxanthine. 100 μl of the resulting suspension was distributed into each well of columns 1-11 of assay microtiter plate leading to a final volume of 125 μl per well, at 1.2% of hematocrit and 0.4% of parasitemia/well.

3. In each plate, 2 columns were reserved for control wells:
Column 11 (comprising wells A11-H11): Positive control wells: Untreated PRBCs to compare with cultures treated with test compounds.
Column 12 (comprising wells A12-H12):
Background value wells: Uninfected RBCs—blank control to obtain the background reading from RBCs without parasites (at 1.2% of hematocrite value).

4. The plates were incubated at 37° C. under low oxygen atmosphere. After 48 hours of incubation 25 μl of radiolabelled [³H] hypoxanthine suspension (Amersham Biosciences, Ref. TRK74) (prepared in pre-warmed complete medium without hypoxanthine at 0.008 mCi/ml and yielding a final concentration of 0.2 μCi/well) were added in each well and the plates were incubated during 24 additional hours. Incorporation was stopped by freezing the plates overnight at −80° C.

5. The growth was quantified by measuring the level of incorporation of [³H]-hypoxanthine into the nucleic acids of the parasite. After thawing the plates, the content of the wells was harvested on glass fibre filters (Wallac, cat ref: 1450-421) with a semi-automated cell-harvester (Harvester 96, TOMTEC). The filters were dried and treated with a Melt-on scintillator (Meltilex® A, PerkinElmer cat ref.: 1450-441). Incorporation of radioactivity was measured with a β-counter (Wallac Microbeta, PerkinElmer).

The assays were repeated at least three independent times.

### V. Analysis of the Data

The value of each well was corrected by subtracting the background value from the absolute value. Background was calculated for each plate as the average value in counts per minute (cpm) of the uninfected control wells.

For each concentration of each test compound, the percentage of inhibition was then calculated by comparison with the value obtained from a control wells (average value of cpm from wells located in column 11) containing untreated PRBCs.

For each compound, non-linear regression fit (sigmoid dose-response curve) using GaphPad Prism 4.0 software is adjusted to obtain an IC₅₀ value, corresponding to the concentration which inhibits 50% of parasite development.

Results were expressed as the average IC₅₀ value±standard deviation of at least 3 independent experiments performed on different days.

### Evaluation of Pharmacokinetic Parameters

Evaluation of the pharmacokinetic parameters of test compounds may be performed in CD-1 mice following intravenous (IV) and oral gavage (OG) administration with serial tail vein sampling. The pharmacokinetics and bioavailability test compounds are evaluated after a single intravenous or oral dose to male CD-1 mice. Serial blood samples are collected from each animal via the tail vein. Blood levels of the test compounds are then determined by LC/MS/MS. Following analysis, pharmacokinetic parameters such as DNAUC, Cmax, half-life, clearance and volume of distribution are calculated.

All dosing solutions are prepared fresh on the day of dosing. Dosing solutions are prepared by direct reconstitution of pre-weighed compounds to a final concentration of 2.5 mg/mL. The same dosing solutions are used for both intravenous and oral gavage administration. Vehicles which may be used for each dosing solution include saline (for up to 10 mg/mL), or a mixture of saline, ethanol and acetic acid (the amount of acetic acid is adjusted for each compound in order to achieve complete dissolution).

The pharmacokinetics of test compounds is evaluated in male CD-1 mice. The study is not blinded. Each route of administration is dosed as N=3. Surgically modified mice (jugular vein catheter for the intravenous dose groups only) are housed one per cage, while non-surgically modified mice are housed up to three per cage. Animals are supplied with water and a commercial rodent diet ad libitum prior to study initiation. Food is then withheld from the animals for a minimum of twelve hours before the study and during the study, until food is returned at four hours postdose. Water is supplied ad libitum. For intravenous dosing, the dosing volume is 2 mL/kg for a total dose of 5 mg/kg, and for oral dosing, the dosing volume is 10 mL/kg for a total dose of 25 mg/kg.

Sampling times are as follows for the IV dosing: 5 and 20 minutes, 1, 3, 6, 24 and 30 hours postdose and sampling times are as follows for the OG dosing: 15 and 30 minutes, 1, 3, 6, 24 and 30 hours postdose.

At each timepoint up to 24 hours, 25 μL of blood is collected from the tail vein. At the 30 hour timepoint, samples are collected by cardiac puncture. Each 25 μL blood sample is then placed in a labeled polypropylene tube, and 25 μL of HPLC water is added. These samples are briefly vortexed and then stored in a freezer set to maintain −60° C. to −80° C. Samples remain frozen until thawed for analysis.

Samples are analyzed using the precipitation method (acetonitrile containing an internal standard—100 ng/mL clarithromycin). In brief, the test compounds are extracted from the blood samples via acetonitrile precipitation and analyzed by LC/MS/MS. To determine accuracy and precision, the analytical method for each test compound is subjected to a one-day pre-study validation run. All concentrations are expressed as the free base concentration. A dilution factor of 2 is applied to all samples to account for the addition of HPLC water during sample collection from the animals.

One standard curve, with a minimum of eight points per curve, and a minimum of six quality control samples (QCs) at three concentrations are dispersed throughout each analytical run. At least ⅝ standards are to be within ±20% of nominal except at the lower limit of quantitation, which is to be within ±25% of nominal. Standards not meeting accuracy criteria are removed from the calibration curve. All QCs are to be within ±20% of nominal to be accepted. At least ⅔ of the batch QCs and at least one at each level must pass the acceptance criteria in order for the run to be accepted.

Individual blood concentrations versus time data for each test compound are subjected to non-compartmental analysis using the pharmacokinetic program WinNonlin v. 4.1 software (Pharsight Co., Mountain View, Calif. 94040). Nominal dosing solution concentrations are used in all calculations.

### In Vivo Malaria Mouse Screen

### Thompson Test

The modified Thompson Test in vivo mouse screen tests compounds for blood schizonticidal activity.

The Thompson test is performed as follows: four-five week old male CD-1 mice weighing 16-17 g, purchased from Charles River, are placed 5 per cage and allowed to acclimate for 4-7 days before being infected. They are maintained at 24° C. with a 12 hour light and 12 hour darkness cycle. The mice are fed a standard Ralston Purina™ mouse chow and given water ad-libidum. The cages, corncob bedding, and water bottles are changed biweekly.

Plasmodium berghei, KBG 173 strain, drug-sensitive, is used to infect mice (6 mice per group) on day 0 of the experiment. Non-treated controls are run with every experiment. Positive control groups are included as needed.

Each compound is ground with a mortar and pestle. The formulation of the compound will depend on the compound dissolving properties and the route of compound administration. The procedures required to run an in vivo drug screen extend over 31 days. All days listed below are relative to Day 0, the day the test mice are infected with the malaria parasite.

At day 0 animals are weighed using the In vivo Manager application. The application, running on a Windows 2000 notebook computer, interfaces directly with a balance connected to the computer's serial port. The mice are inoculated intraperitoneally with 5×10⁴ erythrocytes infected with a drug-sensitive strain of Plasmodium berghei (KBG 173 strain). The inoculum is obtained from a donor mouse having a parasitemia between 5-10%. At days 3, 4, and 5 animals are weighed and compounds are administered either PO or SC according to the weight of the mice. Test compounds are given in a volume of 10 mL/Kg of mouse weight. Compounds are administered twice a day, 6 hours apart, for three days starting on the third day post-infection. At day 6, blood smears are taken and parasitemia is determined using Giemsa staining and animals are weighed. At day 7 animals are weighed. On days 10, 13, 17, 20, 24, 27, and 31 blood smears are taken and animals weighed. Mice losing more that 20% of body weight at any time during the study are euthanized.

### Definitions and Test Result Interpretation of the Thompson Test:

Mean Survival Time Controls (MSTC) is the mean day of survival, post-infection, for the infected, nontreated, negative control group.

Mean Survival Time Treated (MSTT) is the mean day of survival, post-infection, for infected, treated groups at each experimental compound at dose level.

Maximum Tolerated Dose (MTD) is the highest daily dose with no toxic deaths. A toxic death is an animal that: dies or is sacrificed before the MSTC (Mean Survival Time Controls), is judged to be a toxic death by the investigator. If toxic deaths are not recorded, MTD is reported as greater than the highest dose tested. If one or more animals dies of toxicity at the lowest dose tested, MTD is reported as less than the lowest dose.

Minimum Active Dose (MAD) is the lowest daily dose that extends the mean survival time by a factor of two relative to the MSTC. To calculate the MAD, the mean survival times for the control (MSTC) and test animals (MSTT) are calculated for each dosage group. The MSTT values for each dose are compared to the MSTC. The lowest dose with an MSTT that equals or exceeds the MSTC×2 is considered the Minimum Active Dose. Animals dying from toxicity are excluded from this analysis.

Minimum Curative Dose (MCD) is the lowest daily dose that cured at least one animal based on the blood examination taken the last day of the experiment (i.e. day 31 for the Thompson Test): cured animals have negative blood smears on the last day of the experiment, failed animals have positive blood smears on the last day of the experiment. If none of the animals are cured, the MCD is reported as more than the highest dose tested. If all animals are cured, MCD is reported as less than the lowest dose tested.

Curative Dose 50 is the daily dose of compound that cures 50% of the mice. Animals that have a negative blood smear on the last day of the experiment are considered cured. If a particular daily dose cured 50% of the animals, the dose is reported as the CD50. Otherwise, the CD50 is estimated from linear regression using the ‘dose-percent survivors’ pairs in the experiment. If survivorship in all groups is <50%, the CD50 is reported as greater than the highest dose tested. If survivorship in all groups is >50%, the CD50 is reported as less than the lowest dose tested. Only animals that die from malaria are included in this analysis.

Suppressive Dose 50 and 90 are the daily dose of compound that suppresses parasitemia by 50% and 90% relative to the infected non-treated controls. The SD50 is determined from data collected on day 6 post infection after three days of treatment and before the negative controls start to die. To calculate an SD50 and SD90, the mean percent parasitemia for the infected, non-treated controls is calculated and this value is normalized to 100% using a constant (Mean % Parasitemia×C=100%). The day 6 parasitemia for each animal is then normalized by multiplying it by C. These Dose-Normalized pairs are analyzed by non-linear regression to determine the SD50 and SD90. Since the sample size in animal tests is often small, the regression analysis is only run if: (a) there are at least four animals with suppression values <50%, if this condition is not met, the SD50 and SD90 values are reported as greater than the maximum dose of compound tested; (b) there are at least four animals with suppression values >=50%, if this condition is not met, the SD50 and SD90 values are reported as less than the maximum dose of compound tested.

### Presumptive Causal Prophylactic Test

The presumptive causal prophylactic test determines if test compounds have activity against either the sporozoite or exoerythrocytic (EE) stages of Plasmodium yoelii in mice. If all of the sporozoites or EE stages are killed, then blood stream parasites will not appear. If some numbers of these asexual tissue stages are killed then there will be a reduction in parasitemia. The mice eventually self cure and most of the mice survive. The compounds will be listed as either active or inactive based on observed parasitemias.

The presumptive causal prophylactic test may yield false positive results because of the relatively short preerythrocytic stage of the parasite (two days) and the unknown biological half-life of the test compound. It is, therefore, considered to be a test for presumptive activity and a positive result must be confirmed in another system such as Plasmodium cynomolgi in rhesus monkeys.

The presumptive causal prophylactic test is performed as follows: Four to five week old male CD-1 mice weighing 16-17 g, purchased from Charles River, are placed 5 per cage and allowed to acclimate for 4-7 days before being treated and infected. The animals are maintained at 24° C. with a 12 hours light and 12 hours darkness cycle. The mice are fed a standard Ralston Purina™ mouse chow and given water ad-libidum. The cages, corncob bedding, and water bottles are changed biweekly.

Each test compound is ground with a mortar & pestle. Compounds to be administered orally (PO) are suspended in 0.5% hydroxyethylcellulose-0.1% Tween 80. Those given subcutaneously (SC) are suspended in peanut oil. Each compound is prepared at 3 different dose levels.

Plasmodium yoelii 17XNL strain, is used to infect mice that will be used to infect the mosquitoes from which the sporozoites are isolated. An inoculum of 2.5×105 sporozoites per 0.1 mL are used to inoculate the test mice described above, 4 hours after administration of the test compound. The EE stage in the liver exists for only two days and the hypnozoite stage does not exist. Mice often self-cure from blood stage infections. Infected, non-drug-treated controls are run with every experiment to validate the viability of the sporozoites. While these sporozoites usually produce patent infections, some mice may remain blood negative. Caution must be taken when judging a compound as prophylactic when the patency rate in the negative controls is less than 100%. The patency rate must be >80% to consider the test successful. Positive control groups are included occasionally. Additional control mice are treated with Primaquine or Tafenoquine, which are prophylactically effective against the sporozoite and exoerythrocytic (EE) stages of Plasmodium yoelii.

### In Vivo Malaria Rhesus Presumptive Causal Prophylactic Test

Note: The Rhesus Causal Prophylactic Test, CP, was not implemented until 2001. Neither Sweeney (1991) nor Davidson et al. (1981) mention it.

The Rhesus Presumptive Causal Prophylactic Test is used to determine if test compounds have activity against either the sporozoite and/or exoerythrocytic (EE) stages of Plasmodium cynomolgi in Rhesus monkeys.

Briefly, healthy Indian Rhesus monkeys, Macaca mulatta, weighing 2-4 kg of either sex are used. Efforts are made to obtain an equal sex distribution and to keep animals in each test as uniform as possible. Prior to use, each animal undergoes a quarantine for at least five weeks during which time they are tuberculin-tested and treated with thiabendazole. Only malaria free monkeys are used. Usually, two monkeys are used for each dose.

Monkeys are often used in multiple experiments. If a monkey relapses, its infection is cleared with a radical treatment of primaquine and chloroquine before enrollment into a subsequent experiment.

Prior to administration compounds are dissolved in distilled water. If a compound is insoluble in distilled water it is solubilized in methylcellose, DMSO or HEC Tween. Drug concentrations are based on the body weight of each monkey which is determined the day before the first treatment.

Sporozoites of Plasmodium cynomolgi bastianelli isolated from laboratory infected mosquitoes are used for infection (see method below). Monkeys are infected with 0.5-1.5×106 sporozoites intravenously on day 0. Experimental monkeys are treated with compound on days −1, 0, and 1 relative to the day of infection. Negative control monkeys are given vehicle on the same days. To determine parasitemia, thin films are made from peripheral blood and stained with Giemsa. The number of infected RBCs per 500 RBCs is determined and converted to a percentage. If parasites are not found in 500 RBCs, then 1,000 RBCs are counted.

In non-treated monkeys, a rapidly rising parasitemia develops after a 7-9 day prepatent period. The test is considered valid when the controls develop parasitemia. All monkeys that become parasitemic are given a radical treatment with primaquine in combination with chloroquine. Blood smears to determine parasitemia are taken daily through day 20 post infection, and every 2-3 days thereafter.

The criteria for compound activity and toxicity are as follows. A compound is labeled as prophylactic if the monkeys show negative blood films for 30 days after splenectomy, or for 100 days in intact monkeys. The lowest total dose, mg/Kg body weight, resulting in negative blood films is reported as the MCD, Minimum Curative Dose. If the MCD is equal to the lowest total dose tested, then it is reported as ‘<=’ the value of the lowest dose. If the highest dose tested is not prophylactic, then the MCD is reported as ‘>’ the value of the highest total dose.

A compound is not considered prophylactic if parasitemia appears within 30 days post-infection in splenectomized monkeys, or within 100 days post infection in intact monkeys. A compound is toxic if the investigator records a death, sign or symptom from compound toxicity rather than from malaria or an accident. The lowest total dose that causes toxicity is the MTD, Minimum Tolerated Dose. If the MTD is equal to the lowest total dose tested, then it is reported as ‘<=’ the value of the lowest dose. If the highest dose tested is non-toxic, then the MTD is reported as ‘>’ the value of the highest dose.

### Raising and Infecting the Mosquitoes

Cages of non-infected Anopheles dirus are kept in a room maintained at 27° C. They are allowed to feed on non-infected mice to obtain enough blood needed to produce eggs. Jars with wet cotton and moist paper towels are placed in the mosquito cages. Female mosquitoes lay their eggs on the moist paper towels. The eggs are collected and placed in enamel pans containing water. The eggs hatch and develop into larvae. The larvae are fed a liver powder suspension (2.5% liver powder in water). When the pupae have fully developed, they are placed in empty jars, which are then placed in empty mosquito cages. After the adult mosquitoes emerge from the pupal stage the jars are removed. The mosquito cages containing mosquitoes to be infected are transferred to a room maintained at 21° C. The female mosquitoes are allowed to feed on an anesthetized Rhesus monkey with circulating gametocytes of P. cynomolgi.

The mosquitoes are maintained in this cool room for 17 days and then are taken for sporozoite isolation. During the last 4 days a solution of PenStrep is fed to the mosquitoes to kill as many bacteria in their guts as possible. These mosquitoes are then aspirated into a plastic bag that is heat-sealed. This bag is placed on a freezing table to immobilize the mosquitoes. The bag is opened and the female mosquitoes are collected while the males are discarded.

The infected females are ground with a mortar and pestle in a 1:1 monkey serum-saline solution. Twenty more mL of saline is added to the mortar and the suspension is filtered to remove large pieces of mosquitoes. The sporozoites in the saline suspension are then counted and diluted to get an inoculum of 2.5×105 sporozoites per 0.1 mL. This is then inoculated intravenously into the test monkeys on day 0.

### In Vitro Inhibition of Liver-Stage Development Assay

The Inhibition of Liver-Stage Development Assay (ILSDA) is an in vitro model for evaluating the efficacy of drugs against the exoerythrocytic stages of Plasmodium sp. in the liver. A modification of the method described by Sacci J B, 2002, Methods in Molecular Medicine, Vol 72, Malaria Methods and Protocols, p. 517-520 may be used. To enhance visualization of exoerythrocytic liver stage parasites, a clonal line of P. berghei (PbFluspo) that was stably transformed with green fluorescent protein (GFP), an autonomously fluorescent marker [Natarajan et al., Cellular Microbiology, 3 (6) (2001), 371-379] is used. Sporozoites obtained from mosquitoes infected with the P. berghei-GFP are used to infect a human hepatocellular carcinoma cell line, HepG2, at a 1:1 ratio in 8-well LabTek chamber slides. After a three-hour incubation to allow for invasion, the HepG2 cells are washed to remove sporozoites that have not invaded. The cultures are then treated with test compounds at 3 doses (ten-fold serial dilutions) for 48 hrs, and liver stage parasites are counted by fluorescence microscopy. Percent parasite inhibition is determined as follows: (control GFP count−experimental GFP count/control GFP count)×100. Primaquine, a known causal prophylactic drug, is run simultaneously as a positive control.

### EXAMPLES

The following abbreviations are used in the text: DCM for dichloromethane, DMSO for dimethyl sulfoxide, EtOAc for ethyl acetate, MeOH for methanol, EtOH for ethanol and THF for tetrahydrofuran, PS-CDI for N-cyclohexylcarbodiimide-N′-propyloxymethylpolystirene, LiB(CH₂CH₃)₃H for lithium triethylboro hydride, Et₃N or TEA for triethylamine, HOBT for 1-hydroxy benzotriazole hydrate, HOAc for acetic acid, DCC for dicyclohexylcarbodiimide, DBU for 1,8-diazabicyclo[5.4.0.]undec-7-ene, EDC for 1-(3-dimethylaminopropyl)-3-ethylcarbodiimide, EDCxHCl for 1-(3-dimethylaminopropyl)-3-ethylcarbodiimide hydrochloride.

The compounds and process of the present invention will be better understood in connection with the following examples, which are intended as an illustration only and not limiting the scope of the invention. Various changes and modifications to the disclosed embodiments will be apparent to those skilled in the art and such changes and modifications including, without limitation, those relating to the chemical structures, substituents, derivatives formulations and/or methods of the invention may be made without departing from the spirit of the invention and the scope of the appended claims.

Where reactions are described as having been carried out in a similar manner to earlier, more completely described reactions, the general reaction conditions used were essentially the same. Work up conditions used were of the types standard in the art, but may have been adapted from one reaction to another. In the procedures that follow, reference to the product of a Description or Example by number is typically provided. This is provided merely for assistance to the skilled chemist to identify the starting material used. The starting material may not necessarily have been prepared from the batch referred to. All reactions were either carried out under nitrogen or may be carried out under nitrogen, unless otherwise stated.

### Examples

9-deoxo-9-dihydro-9a-aza-9a-homoerythromicin A, 9-deoxo-9-dihydro-9a-aza-3-O-decladinosyl-9a-homoerythromicin A and 9-deoxo-9-dihydro-9a-aza-3-O-decladinosyl-5-O-dedesosaminyl-9a-homoerythromicin A may be prepared by procedure as described in J. Chem. Soc. Perkin Trans. I (1986) pages 1881-1890. 9a-(γ-aminopropyl)-9a-aza-9-deoxo-9-dihydro-9a-homoerythromycin A, 9a-(γ-aminopropyl)-9a-aza-9-deoxo-9-dihydro-3-O-decladinosyl-9a-homoerythromycin A may be prepared by procedure as described in international patent application WO 02/055531 A1. 9a-(γ-aminopropyl)-9a-aza-9-deoxo-9-dihydro-3-O-decladinosyl-5-O-dedesosaminyl-9a-homoerythromycin A may be prepared by procedure as described in international patent application WO 2004/094449 A1.

### Intermediates:

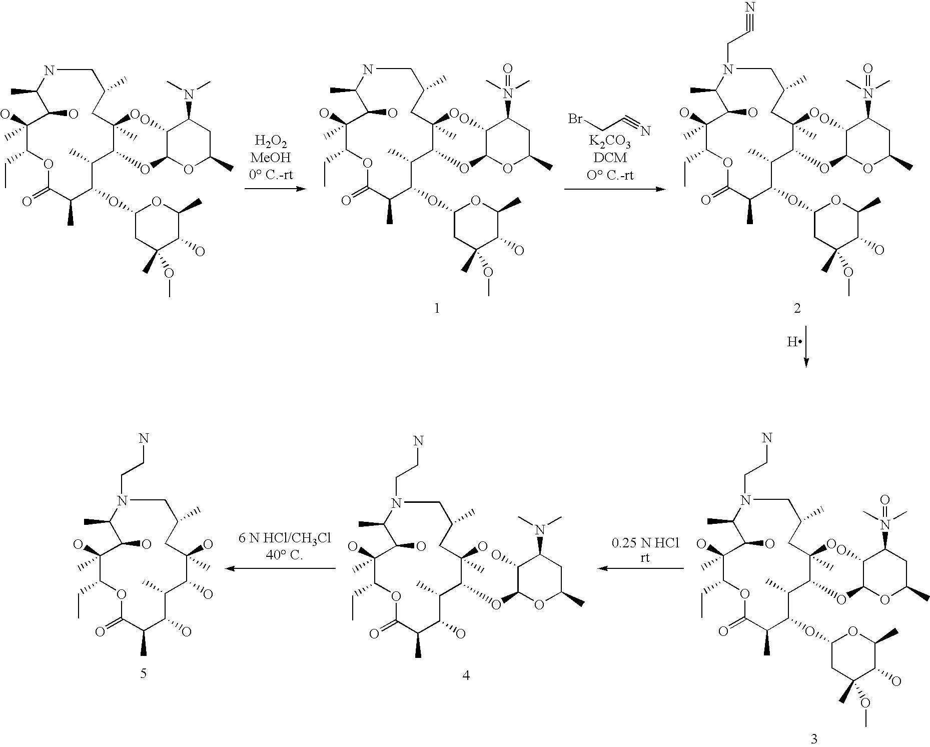

### Intermediate 1

### 9-Deoxo-9-dihydro-3′-N-oxide-9a-aza-9a-homoerythromycin A

To a solution of 9-deoxo-9-dihydro-9a-aza-9a-homoerythromycin A (20 g, 27.21 mmol) in MeOH (80 ml) at 0° C., a 30% water solution of H₂O₂ (30 ml) was added dropwise over 30 min. The reaction mixture was stirred for an additional 1.5 hour at room temperature. After detection of complete transformation the reaction mixture was poured into ice water (400 ml) and DCM (200 ml). A saturated water solution of Na₂S₂O₃ (150 ml) was added to remove excess of H₂O₂. The layers were separated and the water layer extracted with DCM (2×200 ml). Combined organic layers were evaporated under reduced pressure and the residue was precipitated from DCM-diisopropylether yielding the title product (21.5 g, 94.3% yield); MS (ES+) m/z 751.6 [M+H]⁺.

¹³C NMR (125 MHz, pyridine)/δ: 177.3, 101.9, 96.2, 82.8, 77.6, 77.4, 76.9, 75.8, 73.2, 73.0, 72.8, 72.2, 71.9, 65.5, 65.0, 56.2, 55.8, 50.8, 48.6, 44.7, 42.3, 41.9, 34.2, 33.8, 29.1, 27.2, 21.3, 20.8, 20.5, 20.4, 18.3, 16.6, 14.1, 13.5, 10.4, 8.8.

### Intermediate 2

### 9a-Cyanomethyl-9-deoxo-9-dihydro-3′-N-oxide-9a-aza-9a-homoerythromycin A

To a DCM (200 ml) solution of Intermediate 1, (20 g, 26.63 mmol) K₂CO₃ (7.35 g, 53.26 mmol) was added and the reaction mixture was stirred for 10 minutes at room temperature. Then bromoacetonitrile (3.71 ml, 53.26 mmol) was added and the reaction mixture was stirred overnight at room temperature. The reaction mixture was washed with brine yielding after evaporation 20 g of the crude product. Precipitation from water yielded the title product (7.1 g, 31.31% yield); MS (ES+) m/z 790.6 [M+H]⁺.

¹³C NMR (125 MHz, pyridine)/δ: 176.7, 116.9, 101.7, 94.5, 83.3, 77.5, 77.3, 76.7, 75.6, 74.9, 73.8, 73.0, 72.6, 71.4, 65.8, 65.0, 63.2, 60.8, 50.9, 48.6, 44.0, 41.7, 41.5, 36.8, 34.2, 33.7, 30.8, 25.6, 21.2, 20.7, 20.5, 20.4, 20.4, 18.1, 17.1, 13.7, 10.4, 9.1.

### Intermediate 3

### 9a-(β-Aminoethyl)-9-deoxo-9-dihydro-9a-aza-9a-homoerythromycin A

To the solution of Intermediate 2 (3 g, 3.80 mmol) in THF (25 ml), LiB(CH₂CH₃)₃H (10 ml, 1 M THF solution) was added dropwise over 20 minutes at −20° C. The reaction was stirred for 10 minutes at −20° C. to complete conversion. To the reaction mixture water (50 ml) and DCM (50 ml) were added and gradient extraction was performed at pH 4.5 and 10. Evaporation of the combined organic extracts at pH 10 yielded 1.6 g of the crude product. Column chromatography using elution system DCM/MeOH/NH₄OH=90:9:0.5 yielded the title product (0.87 g, 29.5% yield); MS (ES+) m/z 778.5 [M+H]⁺.

¹³C NMR (125 MHz, pyridine)/δ: 177.8, 103.8, 96.9, 84.6, 80.2, 79.2, 78.4, 75.5, 75.4, 75.1, 74.1, 72.3, 70.4, 68.7, 66.6, 66.2, 62.9, 55.0, 50.1, 46.1, 41.9, 41.8, 41.0, 30.9, 30.4, 36.1, 27.6, 23.2, 22.3, 22.1, 20.1, 20.0, 17.7, 16.5, 11.8, 10.9, 9.0.

### Intermediate 4

### 9a-(β-Aminoethyl)-3-O-decladinosyl-9-deoxo-9-dihydro-9a-aza-9a-homoerythromycin A

A solution of Intermediate 3 (1.5 g, 1.93 mmol) in 0.25 N HCl (50 ml) was stirred for 20 hours at room temperature. To the reaction mixture DCM (50 ml) was added and gradient extraction was performed at pH 1.1 and 9.5. Evaporation of the combined organic extracts at pH 9.5 yielded 0.98 g of crude product. Column chromatography using elution system DCM/MeOH/NH₄OH=90:9:1.5 yielded the title product (0.76 g, 62.71% yield); MS (ES+) m/z 620.6 [M+H]⁺.

¹³C NMR (75 Mhz, DMSO)/δ: 174.61, 102.24, 83.30, 76.15, 75.86, 75.20, 73.66, 73.30, 70.16, 67.82, 64.36, 43.63, 40.14, 37.39, 35.84, 30.31, 29.38, 25.67, 21.03, 20.87, 20.66, 16.53, 15.87, 10.27, 8.16.

### Intermediate 5

### 9a-(β-Aminoethyl)-3-O-decladinosyl-5-O-dedesosaminyl-9-deoxo-9-dihydro-9a-aza-9a-homoerythromycin A

A solution of Intermediate 4 (620 mg, 1 mmol) was dissolved in 6 N HCl (20 ml) and CHCl₃ (10 ml) and reaction mixture was stirred at reflux temperature for 40 hours. Gradient extraction was performed at pH 2, 5, 8 and 10.5 with CHCl₃. Evaporation of organic extracts at pH 10.5 gave the title product (300 mg); MS (ES+) m/z 462.9 [M+H]⁺.

### Intermediate 6

### 3-(9-Deoxo-9-dihydro-9a-aza-9a-homoerythromycin A) Propionic Acid Methyl Ester

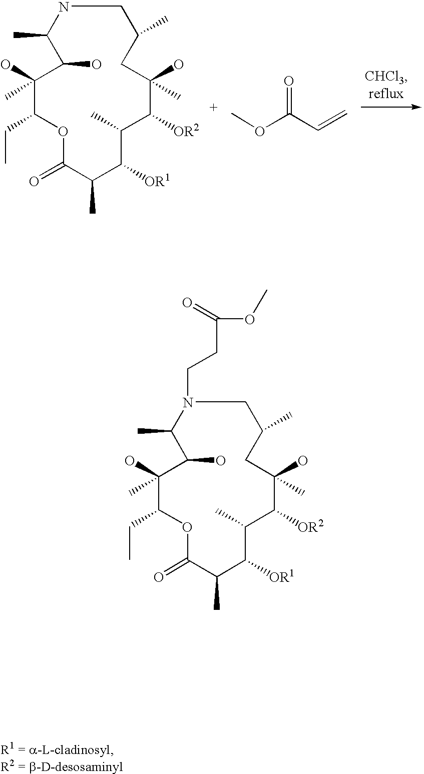

To a solution of 9-deoxo-9-dihydro-9a-aza-9a-homoerythromycin A (4.0 g, 5.44 mmol) in CHCl₃ (80.0 mL) methyl acrylate (24.5 mL, 272.1 mmol) was added. Reaction mixture was stirred under reflux (60° C.) for 2 days. After evaporation of organic solvent crude title product (4.58 g) was obtained.

Crude product was purified using Solid Phase Extraction (SPE) technique on a LC-Si (2 g) cartridge with the FlashMaster II instrument and gradient system for eluation: CH₂Cl₂/(MeOH:NH₄OH=9:1.5) in which MeOH:NH₄OH=9:1.5 was increased from 0 to 12% giving after evaporation of solvent yellowish the title product as powder (2.4 g, 53.7%); MS (ES+) m/z 821.5 [M+H]⁺.

### Intermediate 7

### 3-(9-Deoxo-9-dihydro-9a-aza-9a-homoerythromycin A) Propionic Acid Also Known as 9a-(γ-propionic acid)-9-deoxo-9-dihydro-9a-aza-9a-homoerythromycin A

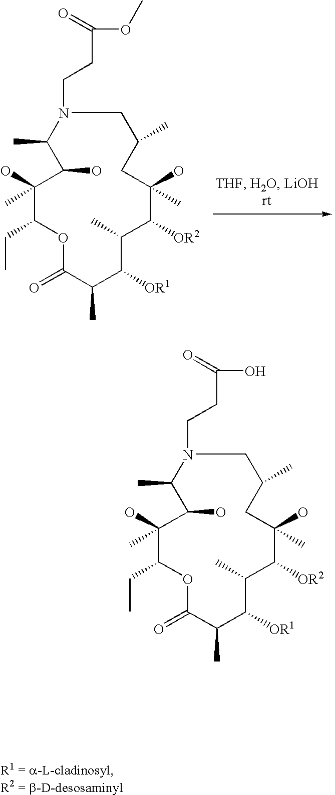

To a solution of Intermediate 6 (2.4 g, 2.92 mmol) in THF (25.0 mL) a solution of LiOH (282.1 mg, 6.72 mmol) in water (25.0 mL) was added. Reaction mixture was stirred at room temperature for 3 hours. Than brine was added to the reaction mixture (30 mL) and extracted with CH₂Cl₂ (3×30 mL). The combined organic extracts were dried over anhydrous Na₂SO₄. After evaporation the title product was obtained (2.30 g, 97.6%); MS (ES+) m/z 807.5 [M+H]⁺.

### Intermediate 8

### 3-(4-Quinolinyl)propanoic Acid

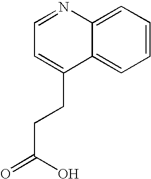

### a) Methyl (2E)-3-(4-quinolinyl)-2-propenoate

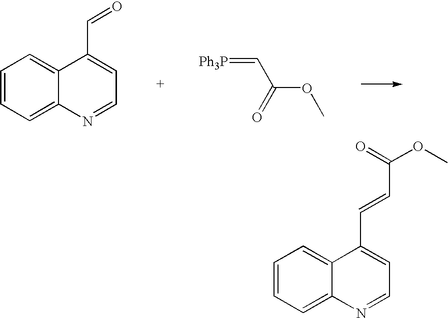

Quinolin 4-carbaldehyde (768 mg, 5 mmol) and methyl(triphenylphosphoranylidene)acetate (1.84 g, 5.5 mmol) were dissolved in toluene (15 mL). Reaction mixture was stirred at 80° C. for 2.5 hours. After cooling to room temperature reaction mixture was extracted with 1N HCl (2×20 mL). Aqueous extracts were washed with EtOAc (15 mL), alkalised with 1N NaOH and extracted with EtOAc (3×15 mL). Combined organic extracts were washed with brine (20 mL), dried over Na₂SO₄ and evaporated under reduced pressure to dryness affording the title product as white solid (1.06 g).

### b) Methyl 3-(quinolin-4-yl)propanoate

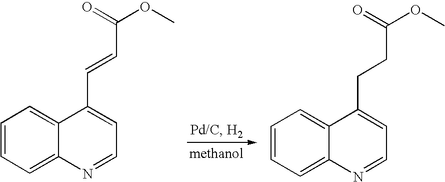

To solution of Intermediate 8a (650 mg, 3 mmol) in methanol (5 mL), 10% Pd/C (40 mg) was added and hydrogenation was performed at low pressure (balloon) over the night. After catalyst was filtered off, filtrate evaporated under the reduced pressure to dryness and the residue purified by column chromatography on silicagel (20 g) using solvent system for elution: hexanes:EtOAc=1:2 to give the title product as colourless oil (536 mg, yield 67%) which can be further purified, if needed by vacuum distillation.

### c) 3-(4-Quinolinyl)propanoic Acid

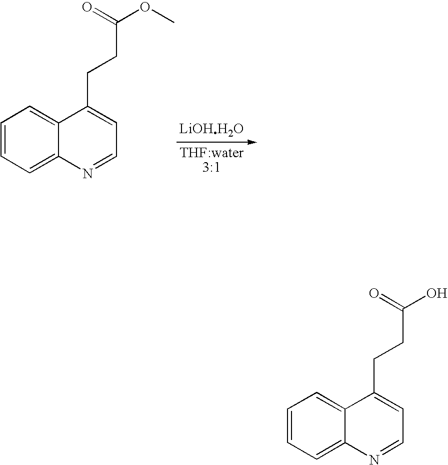

To solution of Intermediate 8b (520 mg, 23.41 mmol) in THF (1 mL), solution of lithium hydroxide monohydrate (111 mg, 2.65 mmol) in a 2:1 mixture of THF:water (3 mL) was added and the reaction mixture was stirred at room temperature overnight resulting in a thick suspension. To obtained suspension a small amount of water was added and stirring continued for approximately 10 minutes until precipitate dissolved. Then brine (10 mL) was added and pH adjusted from 10.5 to 7.04. The aqueous solution was evaporated to dryness and the residue extracted in a Soxlet apparatus using EtOAc as a solvent for 24 hours. Filtrate was evaporated under reduced pressure to dryness and the residue taken in hot acetone. The undissolved material was discarded and the while filtrate was concentrated under the reduced pressure and product triturated with the hexanes giving the title product as a white solid (70 mg, yield 14%); MS (ES+) m/z 202.0 [M+H]⁺.

¹H-NMR (300 MHz) (dmso-d₆) δ 12.5 (bs, 1H); 8.79 (d, J=4.4 Hz, 1H); 8.16 (d, J=8.3, 1H); 8.02 (dd, J=8.4 Hz, J=0.7 Hz, 1H); 7.76 (m, 1H); 7.64 (m, 1H); 7.38 (d, J=4.4 Hz, 1H); 3.35 (m, 2h); 2.71 (t, J=7.7 Hz, 1H).

### Intermediate 9

### (2E)-3-(3-Quinolinyl)-2-propenoic Acid Also Known as (2E)-3-Quinolin-3-yl-acrylic Acid

### a) Methyl (2E)-3-(3-quinolinyl)-2-propenoate

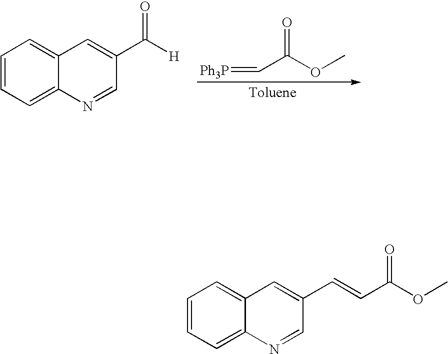

To a solution of 3-quinolinecarboxaldehyde (1.0 g, 6.48 mmol) in toluene (25 ml) (methoxycarbonylmethylene)-triphenylphosphorane (6.5 g, 19.5 mmol) was added and stirred at room temperature for 3 h. To the reaction H₂O (15 ml) and EtOAc (15 ml) were added, layers were separated and the H₂O one extracted twice with EtOAc (15 ml). The combined organic layers were dried, evaporated under reduced pressure and the obtained residue was purified by column chromatography on silicagel using solvent system: EtOAc/n-hexane=1:3 giving the title product (1 g).

### b) (2E)-3-(3-Quinolinyl)-2-propenoic Acid Also Known as (2E)-3-Quinolin-3-yl-acrylic Acid

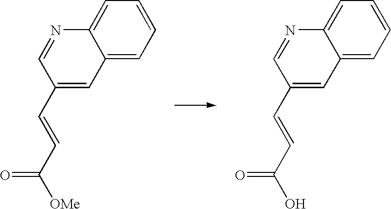

A solution of methyl (2E)-3-(3-quinolinyl)-2-propenoate (100.0 mg, 0.47 mmol) in 1M NaOH (2.0 mL) and EtOH (2.7 mL) was stirred for 2 hours under reflux. After cooling at room temperature, EtOH was evaporated and to the residue water (3.0 mL) and EtOAc (5.0 mL) were added. After separation of layers the pH of aqueous one was adjusted to 2 at which a yellowish precipitate was formed. The precipitate was filtered, washed with water and dried under vacuum at 50° C. giving the title compound (37.9 mg, yield 40.6%); MS (ES+) m/z 200.2 [M+H]⁺.

### Intermediate 10

### 9a-(γ-aminopropyl)-9a-aza-9-deoxo-9-dihydro-9a-homoerythromycin A

### a) 9a-Cyanoethyl-9-deoxo-9-dihydro-9a-aza-9a-homoerythromycin A

The suspension of 9-deoxo-9-dihydro-9a-aza-9a-homoerythromycin A (45 g, 61.23 mmol) in acrylonitrile (166 mL, 2.52 mol) was warmed up to 85° C. and stirred about 22 hours. After cooling the solvent was evaporated under reduced pressure. The residue was suspended three times with toluene and evaporated under reduced pressure giving the title compound (51.24 g); MS (ES+) m/z 788.03 [M+H]⁺.

### b) 9a-(γ-aminopropyl)-9a-aza-9-deoxo-9-dihydro-9a-homoerythromycin A

To a solution of 9a-cyanoethyl-9-deoxo-9-dihydro-9a-aza-9a-homoerythromycin A, Intermediate 10a (51.24 g, 65 mmol) in glacial acetic acid (240 mL), PtO₂ (4.5 g) was added, reaction mixture divided in two reaction vessels and each of them hydrogenated under pressure of 5 bar about 26 hours. The catalyst was removed by filtration and filtrate evaporated under reduced pressure. To the residue water (450 mL) and DCM (250 mL) were added, pH adjusted to 7.3, layers separated, water layer was additionally extracted twice with DCM (200 mL). Then to the water layer DCM (200 mL) was added, pH adjusted to 8.3, layers separated, water layer was additionally extracted twice with DCM (200 mL). The combined organic layers at pH 8.3 were washed with brine, dried over K₂CO₃, evaporated under reduced pressure giving the title compound (29.15 g); MS (ES+) m/z 792.3 [M+H]⁺.

### Examples 1 to 16

### General Procedure

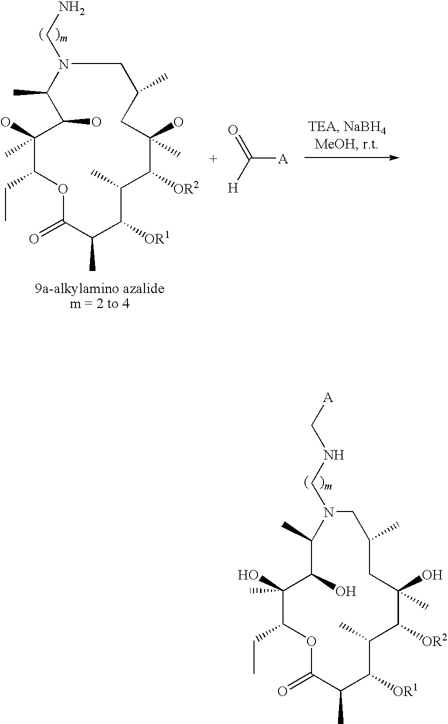

To the degassed solution of corresponding aldehyde (1 equiv.) in MeOH (8 to 30 mL), Et₃N (0.3 equiv.) and 9a-alkylamino azalide (m=2 to 4) (1.2 equiv.) were added and the reaction mixture was stirred at room temperature for 2 hours. Afterwards NaBH₄ (2 equiv.) was added and the reaction mixture was stirred for further 16-24 hours. Solvent was evaporated, the residue dissolved in DCM (20 ml), water (10 ml) was added and the layers were separated. The organic layer was washed with brine (3×20 ml), dried over K₂CO₃ and evaporated under reduced pressure. The crude product was purified using solid phase extraction technique (SPE 5 g) on a LC-Si (2 g) cartridge and gradient system for eluation: DCM/(MeOH:NH₄OH=9:1.5) in which MeOH:NH₄OH=9:1.5 was increased from 0 to 10% giving after evaporation of solvent corresponding compound specified in Table 1.

TABLE 1

| | | | | | | | purity |
|---|---|---|---|---|---|---|---|
| | | | | | | | % |
| | | | | | | | HPLC- |
| | | | | | MS | | MS |
| | | | | | (ES+) | | area |
| Example | m | A | R¹ | R² | m/z | mass/mg | % |
| 1 | 3 | 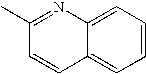 | α-L-cladinose | β-D-desosamine | 933.32[M + H]⁺,calcd.933.25 | 72 | 93.97 |
| 2 | 3 | 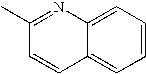 | H | β-D-desosamine | 775.37[M + H]⁺,calcd.778.05 | 120 | 84.89 |
| 3 | 3 | 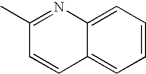 | H | H | 618.19[M + H]⁺,calcd.617.83 | 28 | 94.03 |
| 4 | 3 | 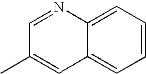 | α-L-cladinose | β-D-desosamine | 933.39[M + H]⁺,calcd.933.25 | 32 | 86.56 |
| 5 | 3 | 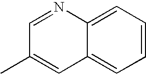 | H | β-D-desosamine | 775.29[M + H]⁺,calcd.775.05 | 80 | 95.65 |
| 6 | 3 | 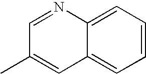 | H | H | 618.21[M + H]⁺,calcd.617.83 | 30 | 88.71 |
| 7 | 3 | 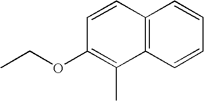 | α-L-cladinose | β-D-desosamine | 976.37[M + H]⁺,calcd.976.31 | 210 | 89.55 |
| 8 | 3 | 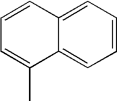 | α-D-cladinose | β-D-desosamine | 933.39[M + H]⁺,calcd.933.25 | 38 | 90.39 |
| 9 | 3 | 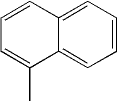 | H | β-D-desosamine | 774.65[M + H]⁺,calcd.774.06 | 85 | 88.43 |
| 10 | 3 | 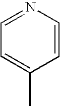 | α-L-cladinose | β-D-desosamine | 883.82[M + H]⁺,calcd.883.19 | 45 | 86.45 |
| 11 | 3 | 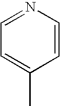 | H | β-D-desosamine | 725.61[M + H]⁺,calcd.724.99 | 53 | 95.62 |
| 12 | 3 | 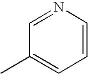 | α-L-cladinose | β-D-desosamine | 883.60[M + H]⁺,calcd.883.19 | 69 | 94.8 |
| 13 | 3 | 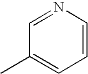 | H | β-D-desosamine | 725.4[M + H]⁺,calcd.724.99 | 52 | 98.1 |
| 14 | 3 | 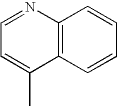 | α-L-cladinose | β-D-desosamine | 933.8[M + H]⁺,calcd.933.25 | 100 | 85.76 |
| 15 | 3 | 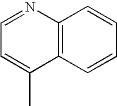 | H | β-D-desosamine | 775.68[M + H]⁺,calcd.775.05 | 150 | 85.43 |
| 16 | 2 | 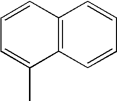 | α-L-cladinose | β-D-desosamine | 918.4[M + H]⁺,calcd.918.23 | 31 | 92.12 |

### Example 1

### 9a-{3-[(Quinolin-2-yl-methyl)amino]propyl}-9-deoxo-9-dihydro-9a-aza-9a-homoerythromycin A

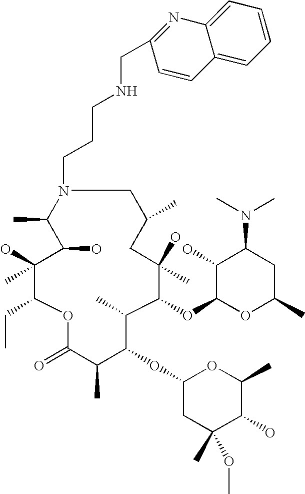

¹³C-NMR (125 MHz, DMSO) δ: 176.0, 146.7, 136.7, 129.7, 128.3, 127.9, 127.0, 126.4, 120.6, 101.6, 95.2, 82.8, 78.3, 77.2, 76.5, 75.5, 74.2, 73.7, 72.6, 70.0, 66.7, 64.9, 64.6, 54.8, 52.8, 48.7, 46.3, 45.3, 44.3, 39.9, 34.8, 30.2, 28.9, 28.2, 27.5, 25.5, 22.2, 21.2, 21.1, 20.9, 18.5, 17.9, 15.2, 10.8, 9.4, 8.9.

### Example 2

### 9a-{3-[(Quinolin-2-yl-methyl)amino]propyl}-3-O-decladinosyl-9-deoxo-9-dihydro-9a-aza-9a-homoerythromycin A

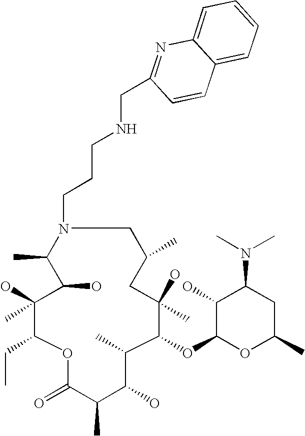

¹³C-NMR (75 MHz, DMSO) δ: 175.3, 160.9, 146.9, 136.08, 129.3, 128.4, 127.7, 126.8, 125.8, 120.6, 103.3, 88.8, 76.3, 76.1, 74.2, 73.2, 70.2, 69.1, 68.3, 65.7, 65.3, 64.4, 55.0, 46.8, 43.8, 36.5, 30.3, 29.4, 28.3, 26.4, 24.5, 21.5, 21.1, 21.0, 20.5, 17.5, 15.5, 10.6, 8.3.

### Example 3

### 9a-{3-[(Quinolin-2-yl-methyl)amino]propyl}-3-O-decladinosyl-5-O-dedesosaminyl-9-deoxo-9-dihydro-9a-aza-9a-homoerythromycin A

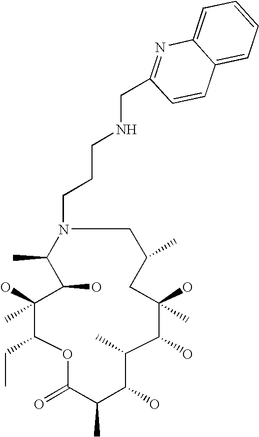

¹³C-NMR (125 MHz, DMSO) δ: 174.9, 161.0, 146.9, 136.1, 129.3, 128.4, 127.7, 126.9, 125.8, 120.6, 81.8, 78.4, 75.9, 74.2, 55.1, 47.1, 43.4, 34.8, 29.7, 28.3, 25.9, 21.7, 21.3, 17.7, 15.5, 10.6, 7.8, 7.3.

### Example 4

### 9a-{3-[(Quinolin-3-yl-methyl)amino]propyl}-9-deoxo-9-dihydro-9a-aza-9a-homoerythromycin A

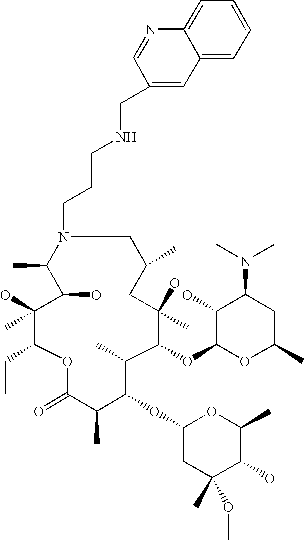

¹³C-NMR (125 MHz, DMSO) δ: 176.1, 151.7, 146.7, 134.4, 133.0, 128.8, 128.6, 127.7, 127.5, 126.5, 101.8, 95.1, 82.6, 78.1, 77.3, 76.6, 75.3, 74.1, 73.6, 72.7, 70.5, 67.0, 64.8, 64.5, 50.3, 48.7, 44.2, 40.2, 34.8, 29.9, 28.9, 28.3, 22.5, 21.3, 21.1, 20.9, 18.4, 17.8, 15.1, 10.8, 9.4, 8.3.

### Example 5

### 9a-{3-[(Quinolin-3-yl-methyl)amino]propyl}-3-O-decladinosyl-9-deoxo-9-dihydro-9a-aza-9a-homoerythromycin A

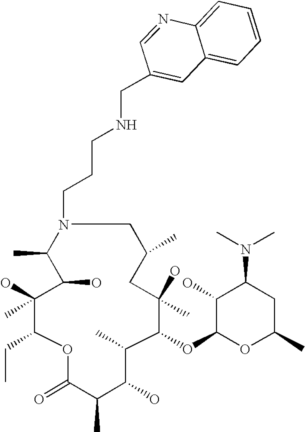

¹³C-NMR (75 MHz, DMSO) δ: 175.3, 151.8, 146.7, 134.3, 133.4, 128.8, 128.6, 127.7, 127.5, 126.4, 103.3, 76.4, 76.1, 74.2, 73.2, 70.2, 68.4, 64.4, 50.5, 46.4, 43.8, 76.4, 76.1, 74.2, 73.2, 70.2, 68.3, 64.4, 54.8, 50.5, 46.5, 43.8, 40.3, 36.6, 30.3, 29.5, 28.2, 26.3, 21.5, 21.1, 21.0, 17.5, 10.6, 8.3, 6.0.

### Example 6

### 9a-{3-[(Quinolin-3-yl-methyl)amino]propyl}-3-O-decladinosyl-5-O-dedesosaminyl-9-deoxo-9-dihydro-9a-aza-9a-homoerythromycin A

### Example 7

### 9a-[3-({[2-(Ethyloxy)-naphthalen-1-yl]methyl}amino)propyl]-9-deoxo-9-dihydro-9a-aza-9a-homoerythromycin A

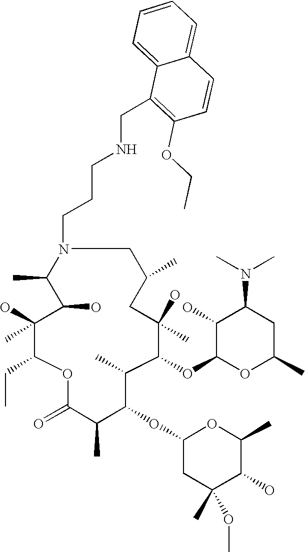

¹³C-NMR (75 MHz, DMSO) δ: 176.1, 154.2, 133.0, 128.7, 128.6, 128.1, 126.2, 123.5, 123.1, 120.8, 114.7, 101.9, 95.1, 82.6, 78.2, 77.3, 76.7, 75.1, 74.2, 73.5, 72.6, 70.6, 67.0, 64.8, 64.5, 64.4, 54.8, 48.7, 46.3, 44.2, 42.0, 40.3, 34.8, 29.9, 28.9, 28.5, 27.2, 26.8, 22.4, 21.4, 21.1, 20.9, 18.4, 17.8, 15.2, 14.9, 10.8, 9.4, 8.5.

### Example 8

### 9a-{3-[(Naphtalen-1-yl-methyl)amino]propyl}-9-deoxo-9-dihydro-9a-aza-9a-homoerythromycin A

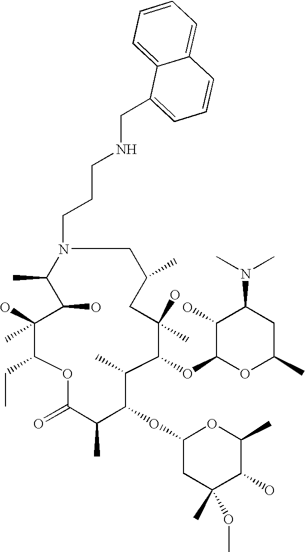

¹³C-NMR (75 MHz, DMSO) δ: 176.2, 135.5, 133.3, 131.4, 128.3, 127.2, 125.9, 125.8, 125.5, 125.3, 123.9, 101.8, 98.3, 95.0, 82.6, 78.0, 77.3, 76.6, 75.2, 74.2, 73.5, 72.6, 70.5, 67.0, 64.8, 64.5, 63.4, 59.3, 54.6, 50.1, 48.7, 48.6, 47.0, 44.2, 40.2, 34.7, 29.9, 28.9, 28.3, 27.1, 26.7, 22.5, 21.4, 21.1, 20.9, 15.0, 10.9, 9.4, 8.6.

### Example 9

### 9a-{3-[(Naphtalen-1-yl-methyl)amino]propyl}-3-O-decladinosyl-9-deoxo-9-dihydro-9a-aza-9a-homoerythromycin A

### Example 10

### 9a-{3-[(Pyridine-4-yl-methyl)amino]propyl}-9-deoxo-9-dihydro-9a-aza-9a-homoerythromycin A

### Example 11

### 9a-{3-[(Pyridine-4-yl-methyl)amino]propyl}-3-O-decladinosyl-9-deoxo-9-dihydro-9a-aza-9a-homoerythromycin A

### Example 12

### 9a-{3-[(Pyridine-3-yl-methyl)amino]propyl}-9-deoxo-9-dihydro-9a-aza-9a-homoerythromycin A

### Example 13

### 9a-{3-[(Pyridine-3-yl-methyl)amino]propyl}-3-O-decladinosyl-9-deoxo-9-dihydro-9a-aza-9a-homoerythromycin A

### Example 14

### 9a-{3-[(Quinolin-4-yl-methyl)amino]propyl}-9-deoxo-9-dihydro-9a-aza-9a-homoerythromycin A

### Example 15

### 9a-{3-[(Quinolin-4-yl-methyl)amino]propyl}-3-O-decladinosyl-9-deoxo-9-dihydro-9a-aza-9a-homoerythromycin A

### Example 16

### 9a-{2-[(Naphtalen-1-yl-methyl)amino]ethyl}-9-deoxo-9-dihydro-9a-aza-9a-homoerythromycin A

### Example 17

### 9a-{3-[Methyl-(naphtalen-1-yl-methyl)amino]propyl}-9-deoxo-9-dihydro-9a-aza-9a-homoerythromycin A

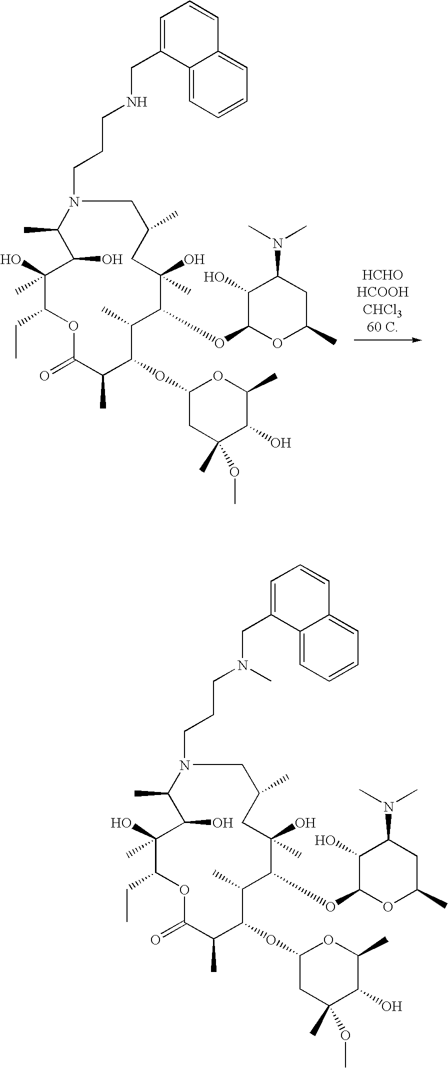

To the solution of Example 7 (0.250 g, 0.268 mmol) in CHCl₃ (3 ml), 36% aqueous solution of formaldehyde (0.041 ml, 0.531 mmol) and HCOOH (0.040 ml, 1.06 mmol) were added and the reaction mixture was stirred at reflux for 22 hours. To the reaction mixture water was added and the pH was adjusted to 10. The layers were separated, the water extracted with DCM and the organic layer was washed with brine, dried over K₂CO₃ and evaporated in vacuum yielding the title compound (0.20 g, Y=70.2%); MS (ES+) m/z 946.66 [M+H]⁺ (calcd. 946.29).

HPLC-MS (area 89.03%).

### Example 18

### 9a-{3-[(Pyridin-2-yl-methyl)amino]propyl}-9-deoxo-9-dihydro-9a-aza-9a-homoerythromycin A

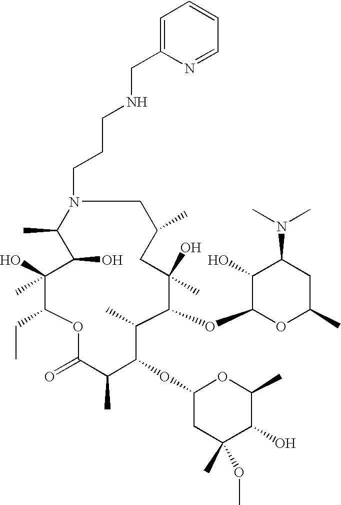

To a solution of pyridine-2-carbaldehyde (11.3 mg, 0.105 mmol) in MeOH (1.50 mL), 9a-(γ-aminopropyl)-9a-aza-9-deoxo-9-dihydro-9a-homoerythromycin A (100.0 mg, 0.126 mmol) was added. Reaction mixture was stirred at room temperature. After 2 hours Pd/C (30 mg) was added and reaction mixture was hydrogenated under pressure of 4.5 bar overnight. The catalyst was removed by filtration over celite and filtrate concentrated in vacuo affording the crude title product (84.0 mg).

Crude product was purified using Solid Phase Extraction (SPE) technique on a LC-Si (2 g) cartridge with the FlashMaster II instrument and gradient system for eluation: CH₂Cl₂/(MeOH:NH₄OH=9:1.5) in which MeOH:NH₄OH=9:1.5 was increased from 0 to 9% giving after evaporation of solvent the title compound as yellowish powder (64.1 mg, 57.6%).

Additional purification was performed on preparative LC-MS (XTerra Prep RP₁₈ column, 5 μm, 19×100 mm) using gradient system for elution: (0.1% HCOOH in H₂O/CH₃CN) in which HCOOH:CH₃CN was changed from 95:5 to 60:40 to give the title compound as formiate salt (11.1 mg); MS (ES+) m/z 883.5 [M+H]⁺ (calculated 883.6).

### Example 19

### 9a-{3-[(3-Phenylpropyl)amino]propyl}-9-deoxo-9-dihydro-9a-aza-9a-homoerythromycin A

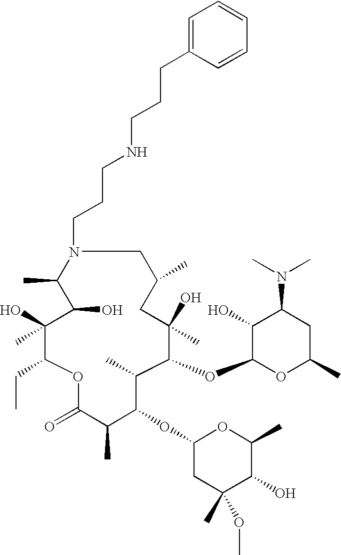

To a solution of 3-phenylpropionaldehyde (13.8 μL, 0.105 mmol) in MeOH (1.0 mL), Et₃N (4.4 μL, 0.032 mmol) and 9a-(γ-aminopropyl)-9a-aza-9-deoxo-9-dihydro-9a-homoerythromycin A (100.0 mg, 0.126 mmol) in MeOH (4.0 mL) were added. Reaction mixture was stirred at room temperature. After 2 hours Pd/C (30 mg) and MeOH (5.0 mL) were added and reaction mixture was hydrogenated under pressure of 3.0 bar overnight.

The catalyst was removed by filtration over celite and filtrate concentrated in vacuo affording the oily title product (120.0 mg).

Crude product was purified using Solid Phase Extraction (SPE) technique on a LC-Si (2 g) cartridge with the FlashMaster II instrument and gradient system for eluation: CH₂Cl₂/(MeOH:NH₄OH=9:1.5) in which MeOH:NH₄OH=9:1.5 was increased from 0 to 9% giving after evaporation of solvent white powder (48.5 mg).

Additional purification was performed on preparative LC-MS (XTerra Prep RP₁₈ column, 5 μm, 19×100 mm) using gradient system for elution: (0.1% HCOOH in H₂O/CH₃CN) in which HCOOH:CH₃CN was changed from 95:5 to 70:30 to give the title compound as formiate salt (8.47 mg, 7.4%); MS (ES+) m/z 910.4 [M+H]⁺ (calculated 910.6).

### Example 20

### 9a-{3-[(2-Phenylethyl)amino]propyl}-9-deoxo-9-dihydro-9a-aza-9a-homoerythromycin A

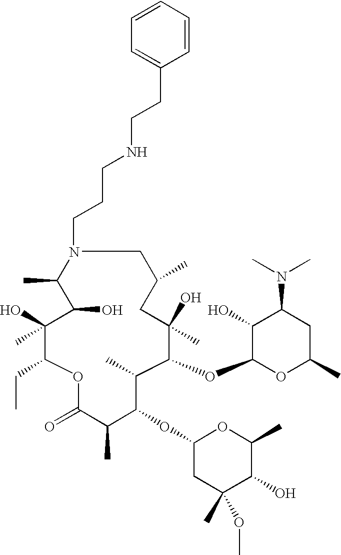

To a solution of phenylacetaldehyde (13.0 μL, 0.126 mmol) in MeOH (1.0 mL), Et₃N (4.4 μL, 0.032 mmol) and solution of 9a-(γ-aminopropyl)-9a-aza-9-deoxo-9-dihydro-9a-homoerythromycin A (100.0 mg, 0.126 mmol) in MeOH (4.0 mL) were added. Reaction mixture was stirred at room temperature. After 2 hours Pd/C (30 mg) and MeOH (5.0 mL) were added and reaction mixture was hydrogenated under pressure of 3.0 bar overnight.

The catalyst was removed by filtration over celite and filtrate concentrated in vacuo affording the oily title product (100.0 mg).

The purification was performed on preparative LC-MS (XTerra Prep RP₁₈ column, 5 μm, 19×100 mm) using gradient system for elution: (0.1% HCOOH in H₂O/CH₃CN) in which HCOOH:CH₃CN was changed from 95:5 to 70:30 to give the title compound as formiate salt (12.4 mg, 10.98%); MS (ES+) m/z 896.5 [M+H]⁺ (calculated 896.6).

### Example 21A

### 9a-{3-[(7-Chloro-quinolin-4-yl)amino]propyl}-9-deoxo-9-dihydro-9a-aza-9a-homoerythromycin A; and

### Example 21B

### 9a-{3-[(7-Chloro-quinolin-4-yl)amino]propyl}-3′N-demethyl-9-deoxo-9-dihydro-9a-aza-9a-homoerythromycin A

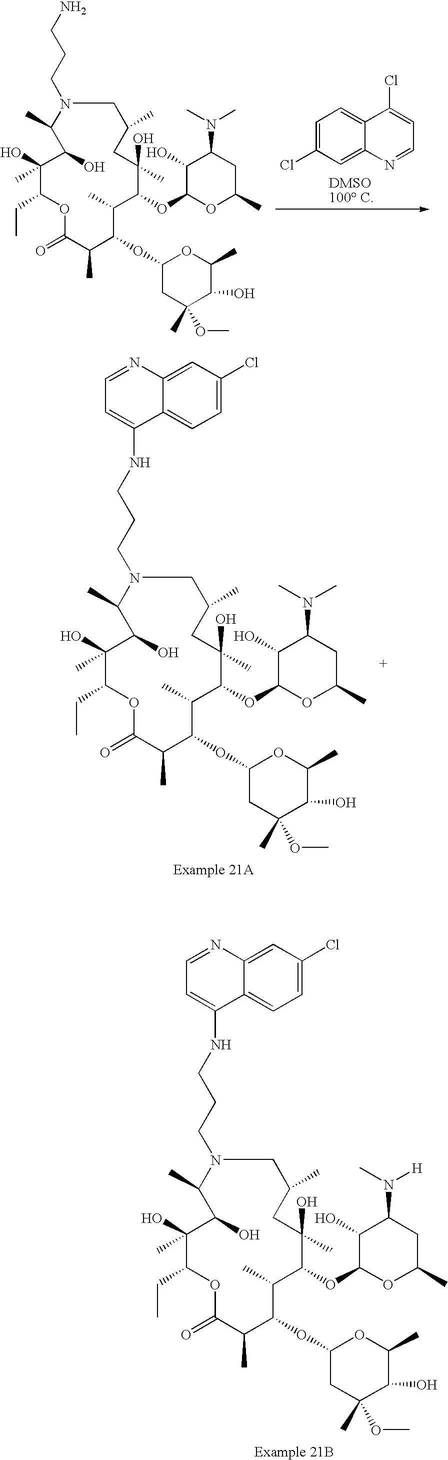

### Method A:

To a solution of 9a-(γ-aminopropyl)-9a-aza-9-deoxo-9-dihydro-9a-homoerythromycin A (4.09 g, 5.05 mmol) in DMSO (12 mL), 4,7-dichloroquinoline (3.0 g, 15.15 mmol) was added. The reaction mixture was stirred at 100° C. for 14 hours and then H₂O (100 mL) and EtOAc (100 mL) were added. The layers were separated (addition of a little amount of 2-propanol aids separation) and the EtOAc layer was washed twice with H₂O (100 mL) and evaporated in vacuum. N-hexane (200 mL) was added to the residue and mixture heated to reflux temperature and then allowed to cool. The precipitated solid was collected by filtration and purified by column chromatography on silicagel using solvent system: DCM:MeOH=7:3 giving Example 21A (86.5 mg) and 55 mg of material which was further purified using solvent system EtOAc:triethylamine=10:0.7 to give a further 44.4 mg of Example 21A and Example 21B (30 mg).

Example 21A was then precipitated from EtOAc:n-hexane yielding 1.2 g.

MS (ES+) m/z 953.71 [M+H]⁺

¹³C-NMR (75 MHz, DMSO-d₆) δ/ppm: 176.3, 152.0, 150.2, 149.3, 133.4, 127.7, 124.3, 124.1, 117.4, 102.0, 98.8, 95.4, 77.5, 76.6, 75.1, 74.4, 72.6, 70.6, 67.1, 64.9, 64.8, 48.9, 48.7, 41.0, 40.5, 34.9, 30.0, 25.7, 21.4, 21.4, 21.0, 18.6, 18.3, 11.0, 9.3.

Example 21B

MS (ES+) m/z 939.65 [M+H]⁺

¹³C-NMR (75 MHz, CDCl₃) δ/ppm: 177.6, 151.8, 150.1, 134.9, 128.5, 125.3, 121.5, 124.1, 117.3, 103.4, 99.0, 96.8, 85.5, 80.1, 78.4, 77.8, 75.3, 75.3, 75.0, 74.7, 72.8, 68.9, 65.9, 64.7, 60.4, 60.0, 49.6, 49.4, 45.7, 41.5, 40.8, 40.5, 37.2, 35.1, 33.2, 29.2, 27.9, 22.6, 21.5, 21.4, 21.1, 18.3, 18.3, 16.6, 15.8, 11.3, 10.2, 8.3.

### Method B (Alternative Preparation of Example 21A):

To a solution of 9a-(γ-aminopropyl)-9a-aza-9-deoxo-9-dihydro-9a-homoerythromycin A (7.5 g, 9.47 mmol) in DMSO (50 mL), 4,7-dichloroquinoline (5.8 g, 29.3 mmol) and diisopropyethyl amine (2.36 mL, 13.2 mmol) were added. The reaction mixture was stirred at 100° C. for 16 hours and then H₂O (500 mL) and EtOAc (300 mL) were added. After layers were separated, EtOAc one was washed twice with H₂O (200 mL), dried over K₂CO₃ and evaporated in vacuum. The residue was precipitated from EtOAc-n-hexane. The precipitated solid was filtered (yielding 4.6 g of precipitate) and purified by column chromatography on silicagel using solvent system: DCM:MeOH:NH₃=90:9:0.5) giving Example 21A (1.02 g);

MS (ES+) m/z 953.71 [M+H]⁺.

### Example 22

### 9a-[3-(Quinolin-4-yl-amino)propyl]-9-deoxo-9-dihydro-9a-aza-9a-homoerythromycin A

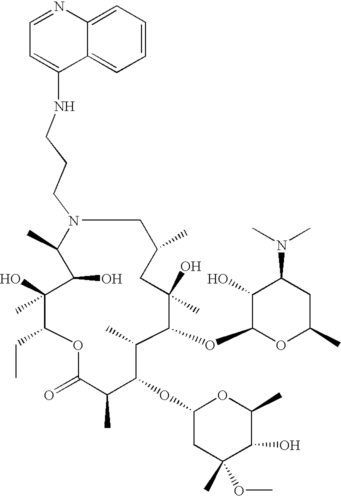

To a solution of Example 21A (50 mg, 0.05 mmol) in EtOH (15 mL), 10% Pd/C (30 mg) was added and the reaction mixture was hydrogenated in Parr apparatus at 4 bar of hydrogen pressure for 24 hours. The catalyst was filtered off over Celite and solvent evaporated under reduced pressure. Product was purified by column chromatography (SP column 5 g, eluent: DCM:MeOH:NH₃=90:9:0.5) yielding the title compound (44 mg); MS (ES+) m/z 919.6 [M+H]⁺.

¹³C-NMR (75 MHz, DMSO-d₆) δ/ppm: 176.4, 150.8, 150.0, 148.3, 129.1, 128.9, 123.7, 122.0, 102.2, 98.4, 95.1, 82.8, 78.1, 77.5, 76.5, 75.1, 74.4, 73.6, 72.8, 70.7, 67.1, 64.9, 64.7, 62.7, 59.8, 48.9, 48.8, 44.3, 41.1, 40.5, 40.4, 40.3, 34.7, 30.0, 27.9, 27.2, 25.8, 22.4, 21.4, 21.3, 20.9, 18.4, 18.1, 14.9, 11.0, 9.4, 9.4.

### Example 23

### 9a-{3-[(7-Chloro-quinolin-4-yl)amino]propyl]}-3-O-decladinosyl-9-deoxo-9-dihydro-9a-aza-9a-homoerythromycin A

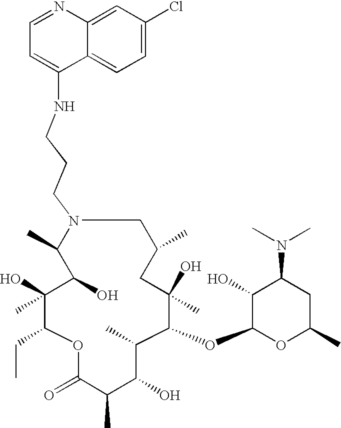

To a solution of 9a-(γ-aminopropyl)-3-O-decladinosyl-9a-aza-9-deoxo-9-dihydro-9a-homoerythromycin A (1.5 g, 1.89 mmole) in DMSO (4.5 mL), 4,7-dichloroquinoline (0.75 g. 3.39 mmole) was added. The reaction mixture was stirred at 100° C. for 16 hours. To the reaction mixture EtOAc (50 mL) was added which resulted in precipitation. The precipitate was removed by filtration, to the filtrate water (50 mL) was added and pH adjusted to 9. The layers were separated and water layer extracted once more with EtOAc. The combined EtOAc layers were evaporated in vacuum giving 1.3 g of residue which was precipitated from EtOAc-n-hexane giving the title product (341 mg) which was further purified by column chromatography on silicagel using solvent system EtOAc/TEA=10:1 (320 mg) and then precipitated from EtOAc:n-hexane yielding the title compound (217 mg); MS (ES+) m/z 795.60 [M+H]⁺.

¹³C-NMR (75 MHz, DMSO-d₆) δ/ppm: 175.2; 151.9; 150.0; 148.9; 133.1; 127.4; 124.4; 123.8; 117.3; 103.7; 98.9; 90.4; 76.6; 76.3; 76.1; 74.4; 73.3; 70.3; 68.4; 64.5; 61.9; 58.1; 50.8; 43.9; 41.1; 40.5; 39.1; 36.6; 30.2; 29.2; 27.1; 26.6; 21.5; 21.3; 21.0; 17.8; 16.1; 10.6; 8.3; 8.0.

### Example 24

### 9a-[3-(Quinolin-4-yl-amino)propyl]-3-O-decladinosyl-9-deoxo-9-dihydro-9a-aza-9a-homoerythromycin A

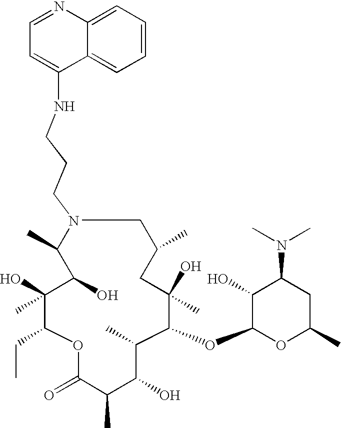

According to the procedure described for Example 22 starting from the Example 23 (0.312 g, 0.39 mmol) the crude title product was obtained. After purification by column chromatography (SP column 10 g, eluent: EtOAc-Et₃N=10:0.7) the title compound was obtained (223 mg); MS (ES+) m/z 761.3 [M+H]⁺.

¹³C-NMR (75 MHz, DMSO-d₆) δ/ppm: 176.4, 150.8, 150.0, 148.3, 129.1, 128.9, 123.7, 122.0, 102.2, 98.4, 95.1, 82.8, 78.1, 77.5, 76.5, 75.1, 74.4, 73.6, 72.8, 70.7, 67.1, 64.9, 64.7, 62.7, 59.8, 48.9, 48.8, 44.3, 41.1, 40.5, 40.4, 40.3, 34.7, 30.0, 27.9, 27.2, 25.8, 22.4, 21.4, 21.3, 20.9, 18.4, 18.1, 14.9, 11.0, 9.4, 9.4.

### Example 25

### 9a-[3-(Quinolin-4-yl-amino)propyl]-3′-N-demethyl-9-deoxo-9-dihydro-9a-aza-9a-homoerythromycin A

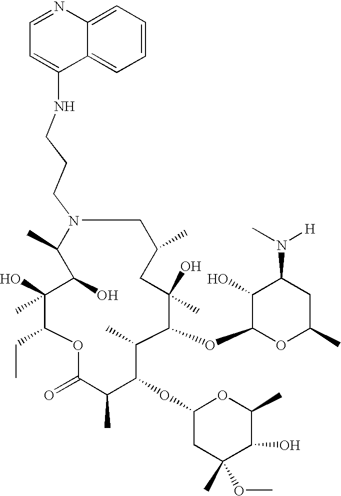

In a solution of Example 22 (0.5 g, 0.54 mmol) in MeOH (20 mL), sodium acetate (0.2 g, 2.7 mmol) and iodine (0.15 g, 0.6 mmol) were added. The reaction mixture was stirred and irradiated with a 500 W halogen lamp for 4 hours, during that time reaction mixture wormed up to 40-50° C. The MeOH was evaporated under reduce pressure, to the residue ethyl acetate (50 mL) and 10% NaHCO₃ solution (50 mL) were added. The ethyl acetate solution was extracted with 10% sodium thiosulphate (3×20 mL), then dried with K₂CO₃, the organic phase was evaporated under reduced pressure and the crude product purified by column chromatography on silicagel using solvent system DCM:MeOH:NH₃=90:9:0.5) to yield the title compound (0.22 g); MS (ES+) m/z 905.4 [M+H]⁺.

¹³C-NMR (75 MHz, DMSO-d₆) δ/ppm: 176.5, 150.8, 150.7, 148.3, 129.0, 128.8, 123.9, 121.8, 118.9, 101.5, 98.0, 95.0, 77.8, 77.4, 76.5, 75.2, 74.4, 73.7, 73.6, 72.8, 66.7, 64.9, 62.7, 60.5, 59.7, 48.8, 48.5, 44.3, 41.0, 40.6, 40.5, 37.5, 34.9, 33.1, 28.1, 27.2, 25.5, 22.4, 21.4, 21.3, 21.0, 18.5, 18.2, 14.8, 11.0, 9.5, 9.3, 8.4.

### Example 26

### 9a-[3-(1H-Purin-6-yl-amino)propyl]-9-deoxo-9-dihydro-9a-aza-9a-homoerythromycin A

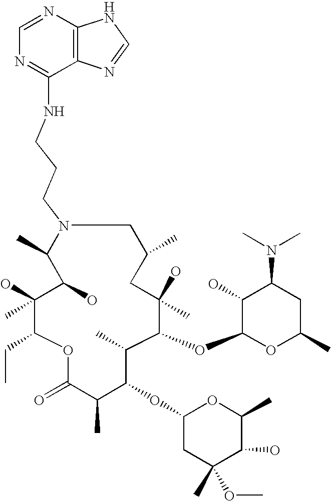

To a solution of 9a-(γ-aminopropyl)-9-deoxo-9-dihydro-9a-aza-9a-homoerythromycin A (0.5 g, 0.63 mmol) in n-butanole (2 mL), 6-chloro-9H-purine (0.56 g, 3.61 mmol) and diisopropylethyl amine (1 mL) were added. The reaction mixture was stirred at 80° C. for 18 hours, than H₂O (100 mL) and DCM (100 mL) were added, and the pH adjusted to 10, the layers were separated and the DCM layer was washed with brine (50 mL), dried over K₂CO₃ and evaporated in vacuum. The residue was precipitated from EtOAc-hexane and purified by column chromatography on silicagel using solvent system DCM:MeOH:NH₃=90:9:0.5 to yield the title compound (304 mg); MS (ES+) m/z 910.3 [M+H]⁺.

¹³C-NMR (75 MHz, DMSO-d₆) δ/ppm: δ(13C)/ppm: 176.7, 152.9, 139.0, 102.1, 95.1, 82.7, 78.0, 77.4, 76.4, 75.4, 74.4, 73.5, 72.7, 70.7, 66.8, 64.9, 64.7, 62.9, 60.5, 49.7, 49.0, 44.4, 41.2, 40.5, 40.4, 39.5, 35.0, 29.9, 28.2, 27.7, 27.3, 22.2, 21.5, 21.3, 21.0, 18.4, 18.2, 15.0, 10.9, 9.5, 9.4.

### Examples 27 to 33

### General Procedure

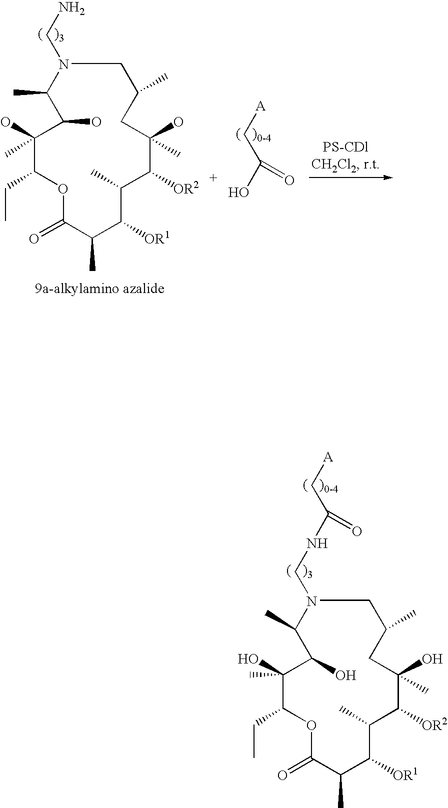 [0407] R¹=α-L-cladinosyl, H
R²=β-D-desosaminyl, H

PS-Carbodiimide resin (PS-CDI, loading: 1.2 mmol/g) (2 equiv) was added to a dry reaction vessel. The corresponding acid (1.5 equiv) dissolved in dry DCM (1.0 mL), was added to the dry resin. The mixture was stirred at room temperature for 1 hour upon which 9a-(γ-aminopropyl)-9-deoxo-9-dihydro-9a-aza-9a-homoerythromycine A (1 equiv) dissolved in dry DCM (0.5 mL) was added. The reaction mixture was stirred for 2 hours at room temperature, filtered and the resin was washed with DCM (4×1.5 mL). The filtrate was evaporated to dryness affording the crude product specified in Table 2 in form of base.

The purification was performed on preparative LC-MS (XTerra Prep RP₁₈ column, 5 μm, 19×100 mm) using gradient system for elution: (0.1% HCOOH in H₂O/CH₃CN) in which HCOOH:CH₃CN was changed from 95:5 to 50:50 to give the compounds specified in Table 2 as formiate salt.

TABLE 2

| | | | Purity | |
|---|---|---|---|---|
| | | | % | |
| Ex- | | MS | HPLC- | |
| am- | | (ES+) | MS | Yield |
| ple | Structure | m/z | area % | % |
| 27 | 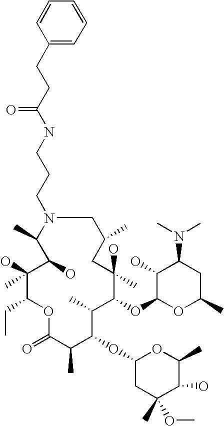 | 924.65[M + H]⁺,calculated924.62 | 96.73 | 24.6 |
| 28 | 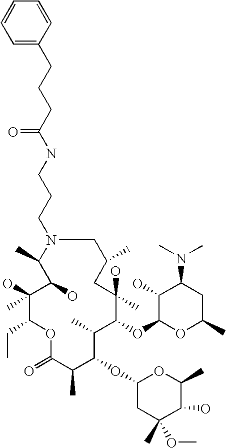 | 938.62[M + H]⁺,calculated938.63 | 95.96 | 37.0 |
| 29 | 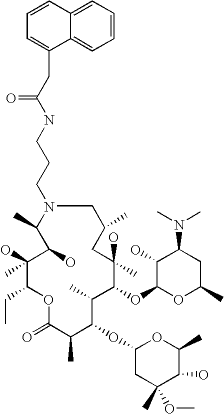 | 960.49[M + H]⁺,calculated960.62 | 98.42 | 43.2 |
| 30 | 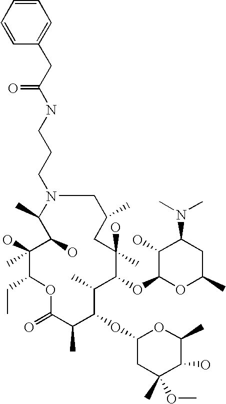 | 910.72[M + H]⁺,calculated910.60 | 97.7 | 42.8 |
| 31 | 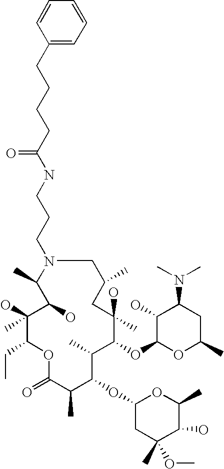 | 952.59[M + H]⁺,calculated952.65 | 99.23 | 48.3 |
| 32 | 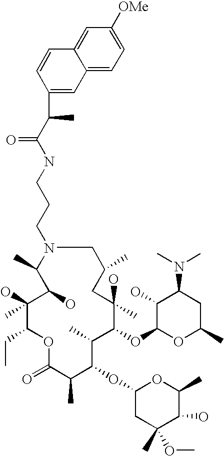 | 1004.60 [M + H]⁺,calculated100.64 | 99.19 | 32.1 |
| 33 | 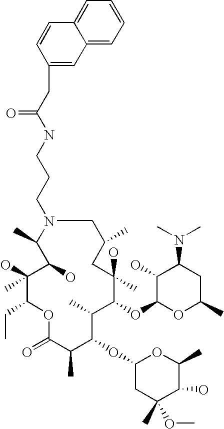 | 960.80[M + H]⁺,calculated960.62 | 95.9 | 41.2 |
| 34 | 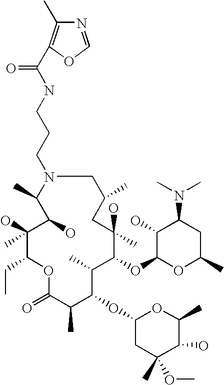 | 901.30[M + H]⁺,calculated901.16 | 95.8 | 38.5 |
| 35 | 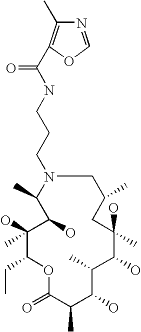 | 585.92[M + H]⁺,calculated585.74 | 94.3 | 29.4 |

### Example 27

### 9a-{3-[(3-Phenylpropanoyl)amino]propyl}-9a-aza-9-deoxo-9-dihydro-9a-homoerythromycin A Formiate Salt

### Example 28

### 9a-{3-[(4-Phenylbutanoyl)amino]propyl}-9a-aza-9-deoxo-9-dihydro-9a-homoerythromycin A Formiate Salt

### Example 29

### 9a-{3-[(Naphtalen-1-yl-acetyl)amino]propyl}-9a-aza-9-deoxo-9-dihydro-9a-homoerythromycin A Formiate Salt

### Example 30

### 9a-{3-[(Phenylacetyl)amino]propyl}-9a-aza-9-deoxo-9-dihydro-9a-homoerythromycin A Formiate Salt

### Example 31

### 9a-{3-[(5-Phenylpentanoyl)amino]propyl}-9a-aza-9-deoxo-9-dihydro-9a-homoerythromycin A Formiate Salt

### Example 32

### 9a-[3-({(2S)-2-[6-(methyloxy)-naphthalen-2-yl]propanoyl}amino)propyl]-9a-aza-9-deoxo-9-dihydro-9a-homoerythromycin A Formiate Salt

### Example 33

### 9a-{3-[(Naphtalen-2-yl-acetyl)amino]propyl}-9a-aza-9-deoxo-9-dihydro-9a-homoerythromycin A Formiate Salt

### Example 34

### 9a-(3-{[(4-Methyl-1,3-oxazol-5-yl)carbonyl]amino}propyl)-9a-aza-9-deoxo-9-dihydro-9a-homoerythromycin A Formiate Salt

### Example 35

### 9a-(3-{[(4-methyl-1,3-oxazol-5-yl)carbonyl]amino}propyl)-3-O-decladinosyl-5-O-dedesosaminyl-9a-aza-9-deoxo-9-dihydro-9a-homoerythromycin A Formiate Salt

### Examples 36 to 37

### General Procedure

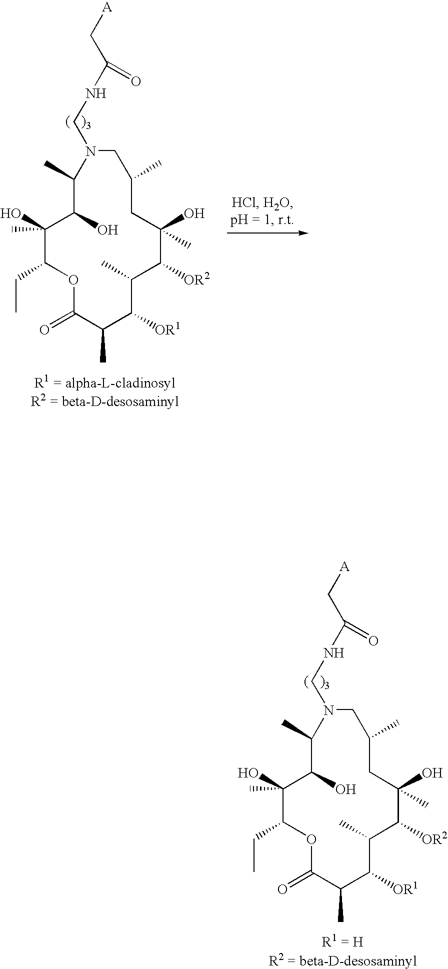

A solution of compound from Example 29 or Example 32 (0.063 mmol), in H₂O (2.0 mL) and 5 wt. % aqueous solution of HCl (3.0 mL) was stirred at room temperature overnight. Then DCM (5.0 mL) was added, pH value adjusted to 9.0 with 0.1 N aqueous solution of NaOH and layers were separated. The water layer was extracted with DCM (3×5 mL), the combined organic extracts washed with brine (10.0 mL), dried over anhydrous Na₂SO₄, filtered and concentrated to give the crude product specified in Table 3 in form of base.

The purification was performed on preparative LC-MS (XTerra Prep RP₁₈ column, 5 μm, 19×100 mm) using gradient system for elution: (0.1% HCOOH in H₂O/CH₃CN) in which HCOOH:CH₃CN was changed from 95:5 to 60:40 to give the compounds specified in Table 3 as formiate salt.

TABLE 3

| | | | Purity | |
|---|---|---|---|---|
| | | | % | |
| Ex- | | MS | HPLC- | |
| am- | | (ES+) | MS | Yield |
| ple | Structure | m/z | area % | % |
| 36 | 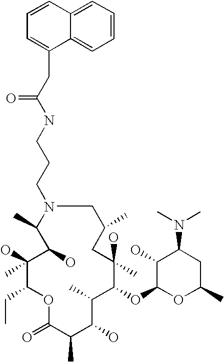 | 802.4[M + H]⁺,calculated802.5 | 99.36 | 47.9 |
| 37 | 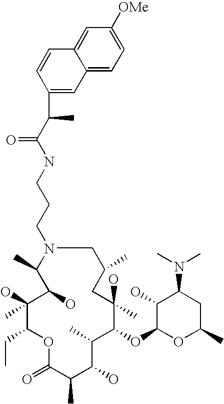 | 846.4[M + H]⁺,calculated846.5 | 98.18 | 58.0 |

### Example 36

### 9a-{3-[(Naphtalen-1-yl-acetyl)amino]propyl}-3-O-decladinosyl-9a-aza-9-deoxo-9-dihydro-9a-homoerythromycin A Formiate Salt

### Example 37

### 9a-[3-({(2S)-2-[6-(methyloxy)-naphthalen-2-yl]propanoyl}amino)propyl]-3-O-decladinosyl-9a-aza-9-deoxo-9-dihydro-9a-homoerythromycin A Formiate Salt

### Examples 38 to 43

### General Procedure

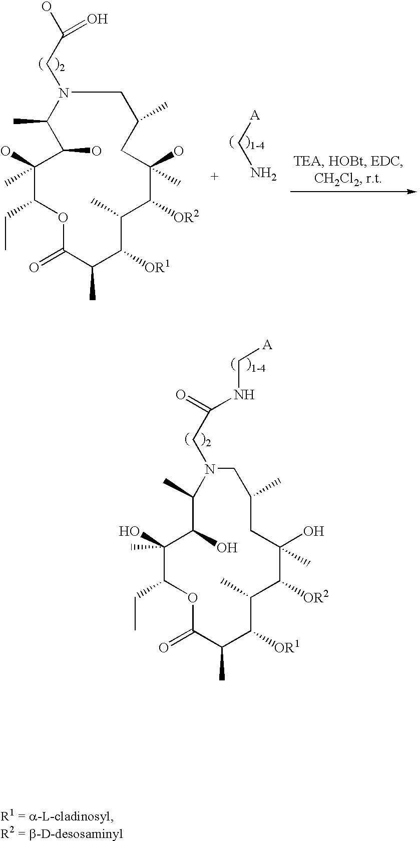

To a solution of Intermediate 7 (36.3 mg, 0.045 mmol) in dry DCM (3.0 mL), TEA (62.4 μL, 0.45 mmol), 1-hydroxy benzotriazole hydrate (HOBt) (12.2 mg, 0.09 mmol), EDC (34.5 mg, 0.18 mmol) and corresponding amine were added (0.0495 mmol). The reaction mixture was stirred at room temperature overnight. The solvent was evaporated to give the crude product specified in Table 4 in form of base.

The purification was performed on preparative LC-MS (XTerra Prep RP₁₈ column, 5 μm, 19×100 mm) using gradient system for elution: (0.1% HCOOH in H₂O/CH₃CN) in which HCOOH:CH₃CN was changed from 95:5 to 60:40 to give the compounds specified in Table 4 as formiate salt.

TABLE 4

| | | | Purity % | |
|---|---|---|---|---|
| | | MS | HPLC-MS | |
| Example | Structure | (ES+) m/z | area % | Yield % |
| 38 | 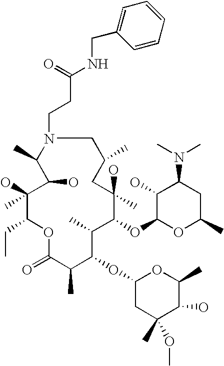 | 896.67[M + H]⁺,calculated896.59 | 93.60 | 34.5 |
| 39 | 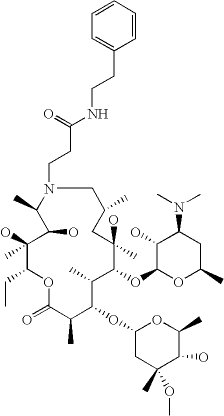 | 910.63[M + H]⁺,calculated910.60 | 96.17 | 40.5 |
| 40 | 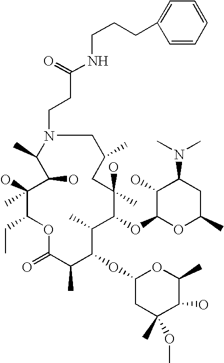 | 924.63[M + H]⁺,calculated924.62 | 96.83 | 39.0 |
| 41 | 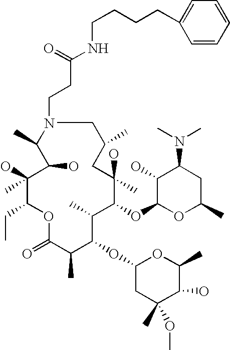 | 938.73[M + H]⁺,calculated938.63 | 91.45 | 38.6 |
| 42 | 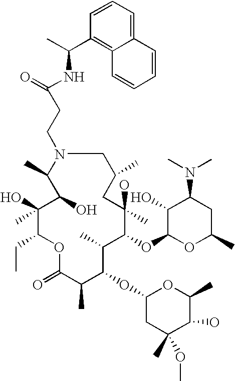 | 960.69[M + H]⁺,calculated960.62 | 97.42 | 34.5 |
| 43 | 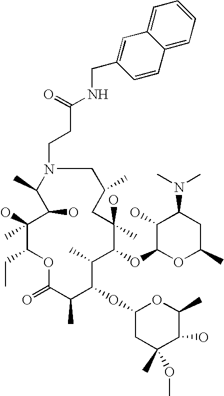 | 946.79[M + H]⁺,calculated946.60 | 94.99 | 38.1 |

### Example 38

### 9a-{1-[(Phenylmethyl)amino]propanoyl}-9-deoxo-9-dihydro-9a-aza-9a-homoerythromycin A Formiate Salt

### Example 39

### 9a-{1-[(2-Phenylethyl)amino]propanoyl}-9-deoxo-9-dihydro-9a-aza-9a-homoerythromycin A Formiate Salt

### Example 40

### 9a-{1-[(3-Phenylpropyl)amino]propanoyl}-9-deoxo-9-dihydro-9a-aza-9a-homoerythromycin A Formiate Salt

### Example 41

### 9a-{1-[(4-Phenylbutyl)amino]propanoyl}-9-deoxo-9-dihydro-9a-aza-9a-homoerythromycin A Formiate Salt

### Example 42

### 9a-{1-[(1S)-1-(1-Naphthalenyl)ethyl]amino)propanoyl}-9-deoxo-9-dihydro-9a-aza-9a-homoerythromycin A Formiate Salt

### Example 43

### 9a-{1-(2-Naphthalenylmethyl)amino]propanoyl}-9-deoxo-9-dihydro-9a-aza-9a-homoerythromycin A Formiate Salt

### Example 44

### 9a-{1-[(1S)-1-(1-Naphthalenyl)ethyl]amino)propanoyl}-9-deoxo-9-dihydro-9a-aza-9a-homoerythromycin A Diacetate Salt

### A) 9a-{1-[(1S)-1-(1-Naphthalenyl)ethyl]amino)propanoyl}-9-deoxo-9-dihydro-9a-aza-9a-homoerythromycin A

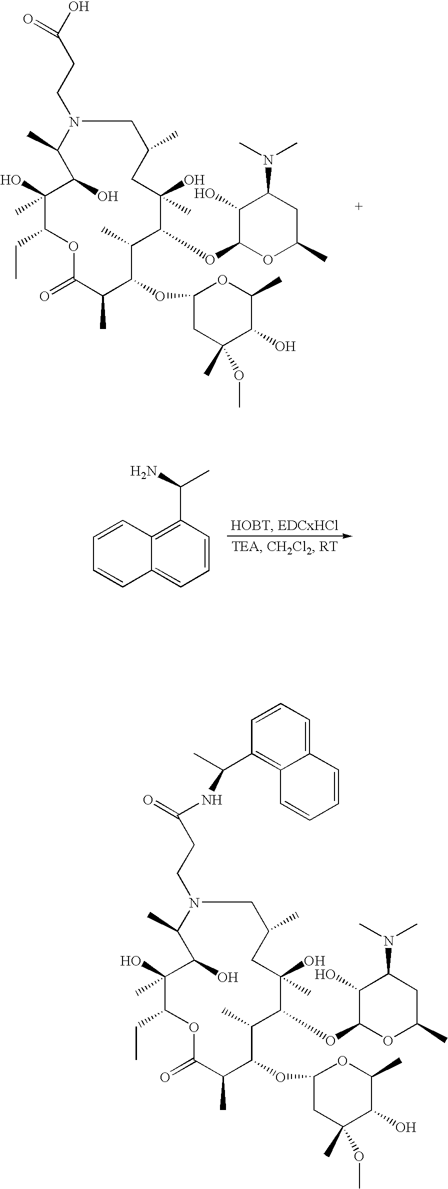

To a solution of 9a-(γ-propanoic acid)-9-deoxo-9-dihydro-9a-aza-9a-homoerythromycin A, Intermediate 7 (1.5 g, 1.86 mmol) in dry DCM (120.0 mL), TEA (2.58 mL, 18.6 mmol), HOBT (502.3 mg, 3.72 mmol), (1S)-1-(1-naphthalenyl)ethanamine (350.1 mg, 2.04 mmol) and EDCxHCl (1.43 g, 7.43 mmol) were added. The reaction mixture was stirred at room temperature overnight. Solvent was evaporated under reduced pressure giving 2.6 g of yellowish crude product which was purified by column chromatography (using solvent system. DCM:MeOH:NH₄OH=90:9:1.5) yielding the title compound (0.953 g, yield 53.4%); MS (ES+) m/z 960.58 [M+H]⁺.

### B) 9a-{1[(1S)-1-(1-naphthalenyl)ethyl]amino)propanoyl}-9-deoxo-9-dihydro-9a-aza-9a-homoerythromycin A Diacetate Salt

To a solution of Example 44A (Example 42 in form of base), (918 mg, 0.956 mmol) in EtOAc (3 mL) glacial HOAc (109.35 μL, 1.912 mmol) was added. After addition of n-hexane (50 mL) the title compound was precipitated as a white solid (969.3 mg, yield 93%); MS (ES+) m/z 960.57 [M+H]⁺.

### Example 45

### 9a-[3-(Quinolin-4-yl-amino)propyl]-9-deoxo-9-dihydro-9a-aza-9a-homoerythromycin A Triacetate Salt

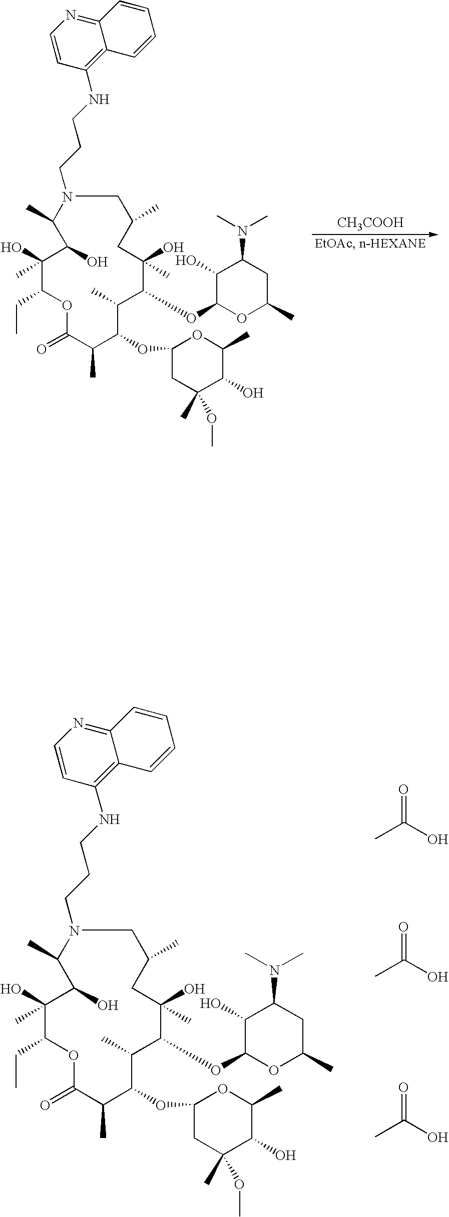

To a solution of 9a-[3-(quinolin-4-yl-amino)propyl]-9-deoxo-9-dihydro-9a-aza-9a-homoerythromycin, Example 22 (1.088 g, 1.18 mmol) in EtOAc (3.0 mL) glacial acetic acid (203.1 μL, 3.55 mmol) was added. After addition of n-hexane (50.0 ml) triacetate salt was precipitated. Precipitate was filtered off and dried under vacuum at room temperature giving the title compound as a white powder (1.13 g, yield 86.8%); MS (ES+) m/z 919.6 [M+H]⁺.

### Example 46

### 9a-{3-[(3-Phenylpropanoyl)amino]propyl}-9a-aza-9-deoxo-9-dihydro-9a-homoerythromycin A

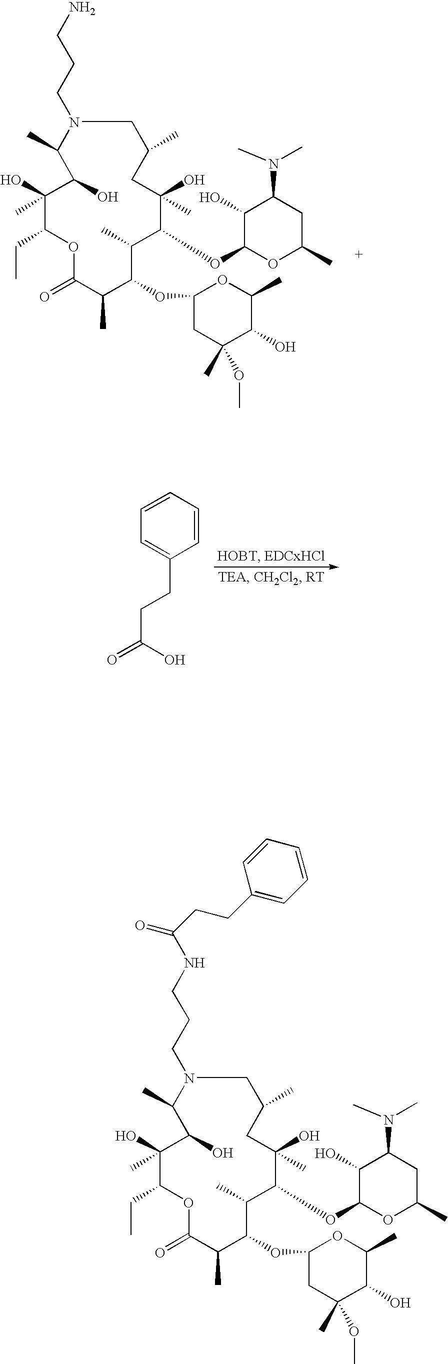

To a solution of 3-phenylpropanoic acid (284.4 mg, 1.89 mmol) in dry DCM (100.0 mL), TEA (2.63 mL, 18.9 mmol), HOBT (511.8 mg, 3.79 mmol), 9a-(γ-aminopropyl)-9a-aza-9-deoxo-9-dihydro-9a-homoerythromycin A (1.5 g, 1.89 mmol) and EDCxHCl (1.45 g, 7.58 mmol) were added and mixture was stirred at room temperature overnight. To reaction mixture water was added (50 mL) and extracted with DCM (3×50 mL). The combined organic extracts were washed with brine (50 mL), dried over anhydrous Na₂SO₄, evaporated under reduced pressure to give yellowish oily residue (2.23 g). Crude product was purified by column chromatography (using solvent system: DCM:MeOH:NH₄OH=90:7:0.5) and by subsequent precipitation from EtOAc/n-hexane giving the title compound (227.1 mg, yield 13.0%); MS (ES+) m/z 924.5 [M+H]⁺.

### Example 47

### 9a-{3-[(3-Phenylpropanoyl)amino]propyl}-9a-aza-9-deoxo-9-dihydro-9a-homoerythromycin A Diacetate Salt

To a solution of compound from Example 46 (642.9 mg, 0.696 mmol) in EtOAc (5.0 mL), glacial acetic acid (79.6 μL, 1.39 mmol) was added. After addition of n-hexane (70.0 ml) triacetate salt was precipitated. Precipitate was filtered off and dried under vacuum at room temperature giving the title compound as a white powder (665.3 mg, yield 91.5%); MS (ES+) m/z 924.5 [M+H]⁺.

### Example 48

### 9a-{1-[(3-Phenylpropyl)amino]propanoyl}-9-deoxo-9-dihydro-9a-aza-9a-homoerythromycin A

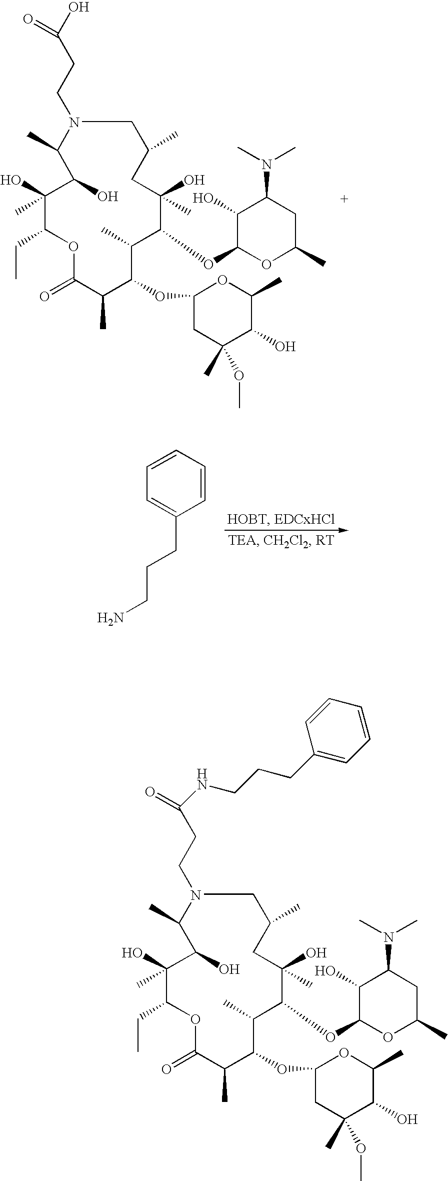

To a solution of 3-phenyl-1-propanamine (315.0 mg, 0.39 mmol) in dry DCM (16.0 mL), TEA (540.6 μL, 3.9 mmol), HOBT (105.4 mg, 0.78 mmol), 9a-(γ-aminopropyl)-9a-aza-9-deoxo-9-dihydro-9a-homoerythromycin A (53.0 mg, 0.39 mmol) and EDCxHCl (299.1 mg, 1.56 mmol) were added. The reaction mixture was stirred at room temperature overnight. The solvent was evaporated under reduced pressure giving yellowish oily residue (0.85 g). Crude product was purified using Solid Phase Extraction (SPE) technique on a LC-SI (20 g) cartridge with the FlashMaster II instrument and gradient system for eluation: DCM:(MeOH:NH₄OH=5:0.5) in which MeOH:NH₄OH=5:0.5 was increased from 0-7% giving after evaporation of solvent the title compound (244.6 mg, yield 67.9%); MS (ES+) m/z 924.4 [M+H]⁺.

### Example 49

### 9a-{1-[(3-Phenylpropyl)amino]propanoyl}-9-deoxo-9-dihydro-9a-aza-9a-homoerythromycin A Diacetate Salt

To a solution of 9a-{1-[(3-phenylpropyl)amino]propanoyl}-9-deoxo-9-dihydro-9a-aza-9a-homoerythromycin A, Example 48 (343.6 mg, 0.372 mmol) in DCM (2.0 mL) glacial acetic acid (42.5 μL, 0.744 mmol) was added. After addition of n-hexane (70.0 ml) diacetate salt was precipitated. Precipitate was filtered off and dried under vacuum at room temperature giving the title compound as a white powder (309.3 mg, yield 79.6%); MS (ES+) m/z 924.5 [M+H]⁺.

### Example 50

### 9a-{3-[(Naphtalen-2-yl-acetyl)amino]propyl}-9a-aza-9-deoxo-9-dihydro-9a-homoerythromycin A

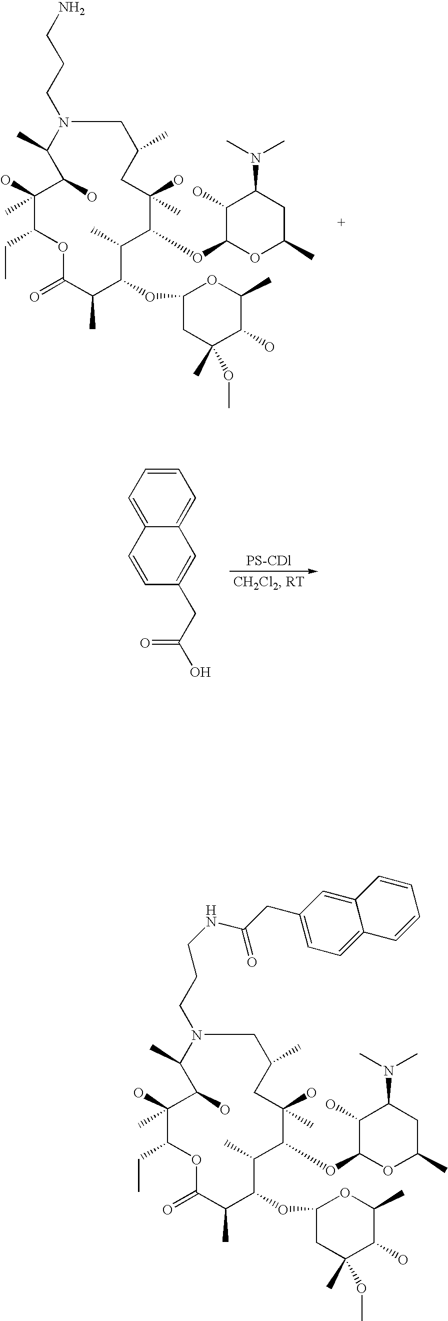

To dry PS-Carbodiimide resin (PS-CDI, loading 1.2 mmol/g) (673.4 mg, 0.81 mmol) 2-naphthalenylacetic acid (122.3 mg, 0.66 mmol) dissolved in dry DCM (10 mL) was added and obtained mixture was stirred at room temperature for 1 hour upon which amine 9a-(γ-aminopropyl)-9a-aza-9-deoxo-9-dihydro-9a-homoerythromycin A, dissolved in dry DCM (5 mL), was added. The reaction mixture was stirred at room temperature for 19 hours. Than additional amounts of 2-naphthalenylacetic acid (28.2 mg, 0.15 mmol) dissolved in dry DCM (5 mL) and PS-CDI (155.3 mg, 0.19 mmol) were added and stirring was continued for 4 hours at room temperature. After fresh amounts of 2-naphthalenylacetic acid (28.2 mg, 0.15 mmol) dissolved in dry DCM (5 mL) and PS-CDI (155.3 mg, 0.19 mmol) were added, stirring was continued for 43 hours at room temperature. The reaction mixture was filtered, resin washed with DCM (3×5 mL) and combined organic solvent evaporated under reduced pressure to give the crude product (463.8 mg). The crude product was precipitated from mixture of solvents EtOAc/n-hexane giving the title compound as a white powder (403.8 mg, yield 84.1%); MS (ES+) m/z 960.5 [M+H]⁺.

### Example 51

### 9a-{3-[(Naphtalen-2-yl-acetyl)amino]propyl}-9a-aza-9-deoxo-9-dihydro-9a-homoerythromycin A Diacetate Salt

To a solution of compound from Example 50 (381.6 mg, 0.397 mmol) in EtOAc (5.0 mL) glacial acetic acid (45.5 μL, 0.795 mmol) was added. After addition of n-hexane (70.0 ml) diacetate salt was precipitated. Precipitate was filtered and dried under vacuum at room temperature giving the title compound as a white powder (373.6 mg, yield 87.1%); MS (ES+) m/z 960.5 [M+H]⁺.

### Example 52

### 9a-{3-[(Phenylmethyl)amino]propyl}-9-deoxo-9-dihydro-9a-aza-9a-homoerythromycin A

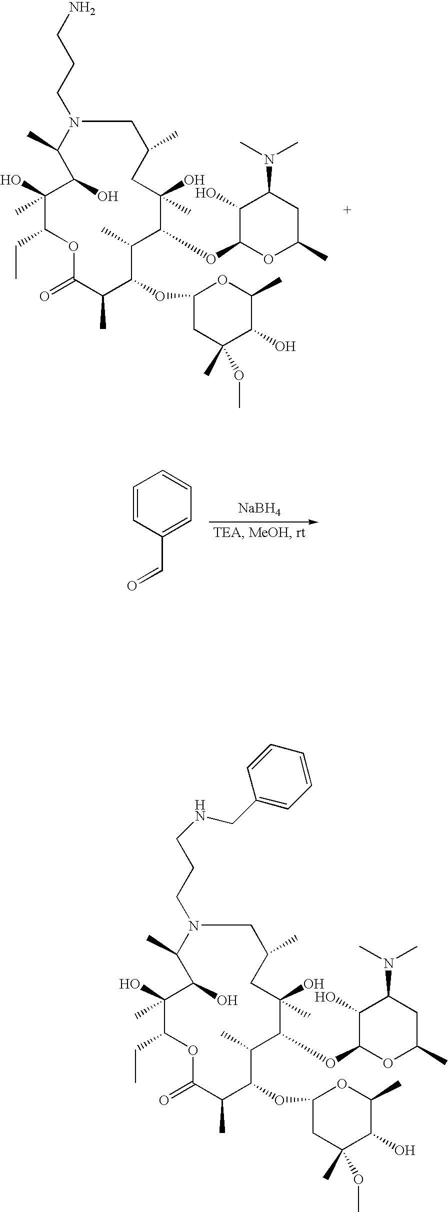

To a solution of benzyl aldehyde (31.9 μL, 0.316 mmol) in MeOH (15.0 mL), Et₃N (13.1 μL, 0.095 mmol) and 9a-(γ-aminopropyl)-9a-aza-9-deoxo-9-dihydro-9a-homoerythromycin A (300.0 mg, 0.379 mmol) were added. After 2 hours NaBH₄ (24.9 mg, 0.63 mmol) was added. Reaction mixture was stirred at room temperature overnight. Than, brine (2.5 mL) and triethanolamine (5.0 mL) were added and the resulting mixture was stirred for 30 minutes. The product was extracted with CH₂Cl₂ (3×15 mL). The combined organic extracts were washed with brine (15.0 mL), dried over anhydrous Na₂SO₄, filtered and concentrated give oily crude product (451.3 mg). The crude product was purified using Solid Phase Extraction (SPE) technique on a LC-Si (10 g) cartridge with the FlashMaster II instrument and gradient system for eluation: DCM/(MeOH/NH₄OH=9/1.5) in which MeOH/NH₄OH=9/1.5 was increased from 0 to 12% giving after evaporation of solvent the title compound (49.0 mg, yield 14.7%); MS (ES+) m/z 882.3 [M+H]⁺; HPLC-MS (area 89.35%).

Additional purification was performed on preparative LC-MS (XTerra Prep RP₁₈ column, 5 μm, 19×100 mm) using gradient system for elution: 0.1% HCOOH in H₂O/CH₃CN in which HCOOH:CH₃CN was changed from 95:5 to 60:40 to give the title product as formiate salt (25.4 mg, yield 7.6%); MS (ES+) m/z 882.5 [M+H]+HPLC-MS (area 90.13%).

### Example 53

### 9a-{3-[(Quinolin-4-yl-methyl)amino]propyl}-3-O-decladinosyl-9-deoxo-9-dihydro-9a-aza-9a-homoerythromycin A Diacetate Salt

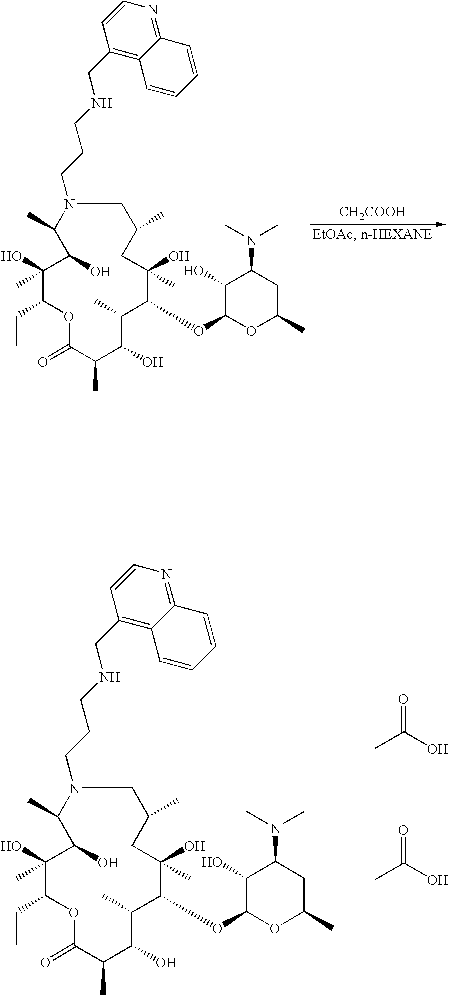

To a solution of 9a-{3-[(Quinolin-4-yl-methyl)amino]propyl}-3-O-decladinosyl-9-deoxo-9-dihydro-9a-aza-9a-homoerythromycin A, compound of Example 15 (562.0 mg, 0.73 mmol) in EtOAc (2.0 mL) glacial acetic acid (82.9 μL, 1.45 mmol) was added. After addition of n-hexane (50.0 ml) diacetate salt was precipitated. Precipitate was filtered and dried under vacuum at room temperature giving the title compound as a white powder (0.49 g, yield 75.5%); MS (ES+) m/z 775.4 [M+H]⁺.

### Example 54

### 9a-{3-[(Naphtalen-2-yl-methyl)amino]propyl}-9-deoxo-9-dihydro-9a-aza-9a-homoerythromycin A

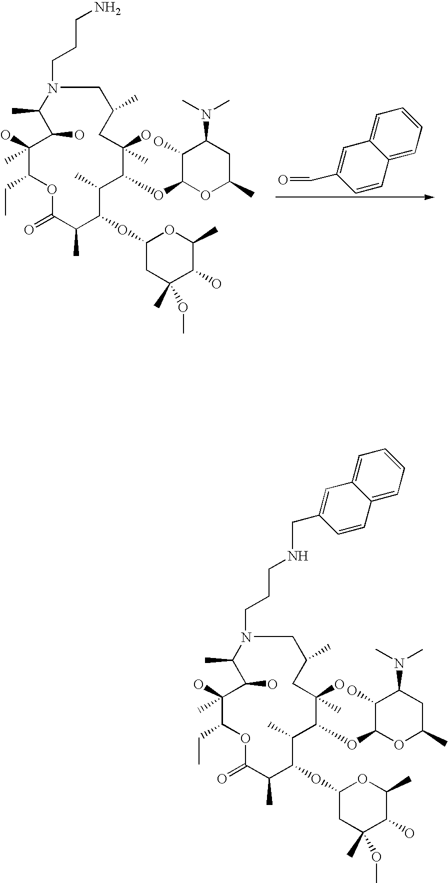

To the degassed solution of 2-naphtaldehyde (82 mg, 0.53 mmol) in MeOH (10 ml) 9a-(γ-aminopropyl)-9a-aza-9-deoxo-9-dihydro-9a-homoerythromycin A (0.5 g, 0.63 mmol) was added and the reaction mixture was stirred at room temperature for 1.5 hour. Afterwards NaBH₄ (40 mg, 1.06 mmol) was added and the reaction mixture was stirred for further 2 hours. Solvent was evaporated, the residue dissolved in DCM (20 ml), water (10 ml) was added and the layers were separated. The organic layer was washed with brine (3×20 ml), dried over K₂CO₃ and evaporated under reduced pressure. The crude product was purified on column chromatography using system for eluation: DCM:MeOH:NH₄OH=90:9:0.5 to give the title compound (0.177 g, yield 28%); MS (ES+) m/z 932.4 [M+H]⁺.

### Example 55

### 9a-{3-[(1,2,3,4-tetrahydro-quinolin-4-yl-methyl)amino]propyl}-9-deoxo-9-dihydro-9a-aza-9a-homoerythromycin A

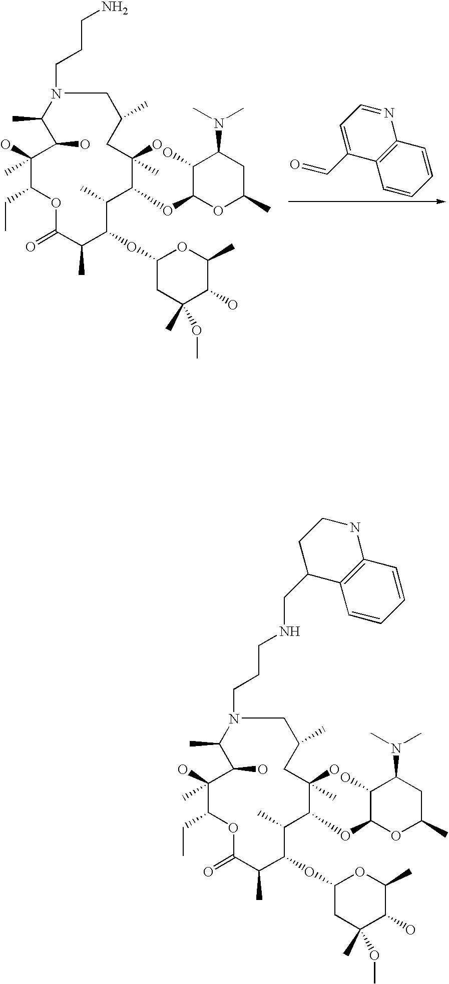

To the degassed solution of 9a-(γ-aminopropyl)-9a-aza-9-deoxo-9-dihydro-9a-homoerythromycin A (2.0 g, 2.22 mmol) in MeOH (50 ml), 4-quinolinecarbaldehyde (0.3 g, 1.85 mmol) and TEA (0.08 ml, 0.56 mmol) were added and the reaction mixture was stirred for 2 hours at room temperature. Than 10% Pd/C (0.5 g) was added and the reaction mixture was hydrogenated in Parr apparatus at 5 barr for 24 hours. After the catalyst was filtered off and solvent evaporated under reduced pressure, the crude product was purified by column chromatography using firstly DCM:MeOH:NH₄OH=90:9:1.5 for eluation and then EtOAc:TEA=10:0.7 yielding the title compound (0.167 g, yield 7%); MS (ES+) m/z 937.6 [M+H]⁺.

### Example 56

### 9a-{3-[(7-Chloro-quinolin-4-yl)amino]propyl]}-3-O-decladinosyl-5-O-dedesosaminyl-9-deoxo-9-dihydro-9a-aza-9a-homoerythromycin A

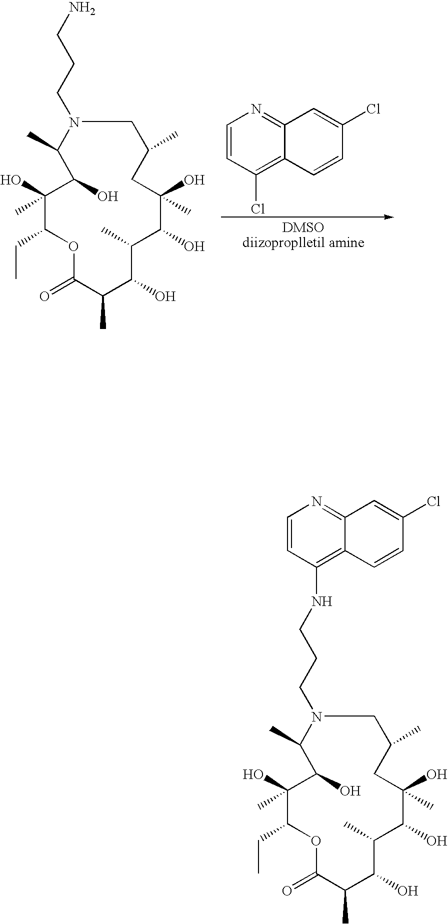

To a solution of 9a-(γ-aminopropyl)-3-O-decladinosyl-5-O-dedesosaminyl-9-deoxo-9-dihydro-9a-aza-9a-homoerythromicin A (1.4 g, 2.94 mmol) in DMSO (20 mL), 4,7-dichloroquinoline (1.8 g, 9.3 mmol) and diisopropyethyl amine (0.6 ml, 3.4 mmol) were added. The reaction mixture was stirred at 100° C. for 18 hours. Than H₂O (100 mL) and EtOAc (100 mL) were added. The solvents were separated and EtOAc layer was washed with H₂O (100 mL), dried over K₂CO₃ and evaporated in vacuum. The residue was precipitated from EtOAc-diisopropyl ether. The precipitated solid was filtered (0.7 g) and purified by column chromatography on silicagel using solvent system: DCM:MeOH:NH₃=90:9:0.5) giving the title compound (0.355 g) as a white powder; MS (ES+) m/z 638.3 [M+H]⁺.

### Example 57

### 9a-(3-{[3-(Quinolin-4-yl)propanoyl]amino}propyl)-9-deoxo-9-dihydro-9a-aza-9a-homoerythromycin A Formiate Salt

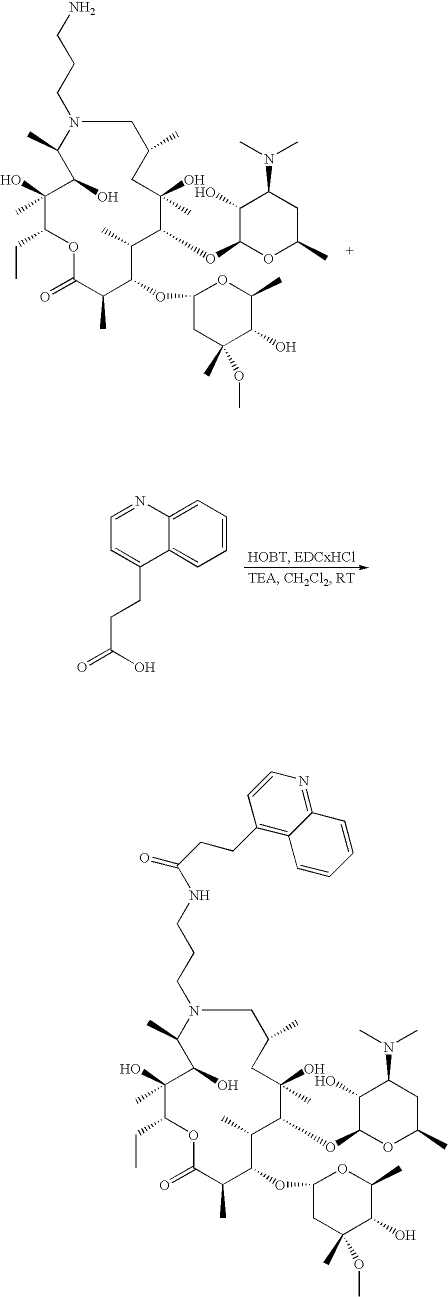

To a solution of 3-(4-quinolinyl)propanoic acid (12.7 mg, 0.063 mmol) in dry DCM (4.5 mL), TEA (87.3 μL, 0.63 mmol), HOBT (17.0 mg, 0.126 mmol), 9a-(γ-aminopropyl)-9a-aza-9-deoxo-9-dihydro-9a-homoerythromycin A (50.0 mg, 0.063 mmol) and EDCxHCl (48.3 mg, 0.252 mmol) were added. The reaction mixture was stirred at room temperature for 24 hours. After evaporation of solvent under reduced pressure the yellowish crude product (130.0 mg) was further purified on preparative LC-MS (XTerra Prep RP₁₈ column, 5 μm, 19×100 mm) using gradient system for elution: 0.1% HCOOH in H₂O/CH₃CN) in which HCOOH:CH₃CN was changed from 95:5 to 60:40 to give the title product as formiate salt (24.8 mg, yield 38.5%); MS (ES+) m/z 975.6 [M+H]⁺.

### Examples 58 to 75

### General Procedure

To dry PS-Carbodiimide resin (PS-CDI, loading: 1.2 mmol/g) (1.29 equiv) dry DCM (1.0 mL) and corresponding acid (1.29 equiv) dissolved in dry DCM (0.1 M solution) were added. The mixture was stirred at room temperature for 1 hour upon which 9a-(γ-aminopropyl)-9-deoxo-9-dihydro-9a-aza-9a-homoerythromycin A (1 equiv) dissolved in dry DCM (0.1 M solution) was added. The reaction mixture was stirred for 2 days at room temperature, filtered and the resin washed with DCM (3×1.0 mL). The combined organic solvent was evaporated under reduced pressure affording corresponding crude product 58-75 specified in Table 5 in form of base. The purification was performed on preparative LC-MS (XTerra Prep RP₁₈ column, 5 μm, 19×100 mm) using gradient system for elution (0.1% HCOOH in H₂O/CH₃CN) in which HCOOH:CH₃CN was changed from 95:5 to 50:40 to give the compounds specified in Table 5 as formiate salt.

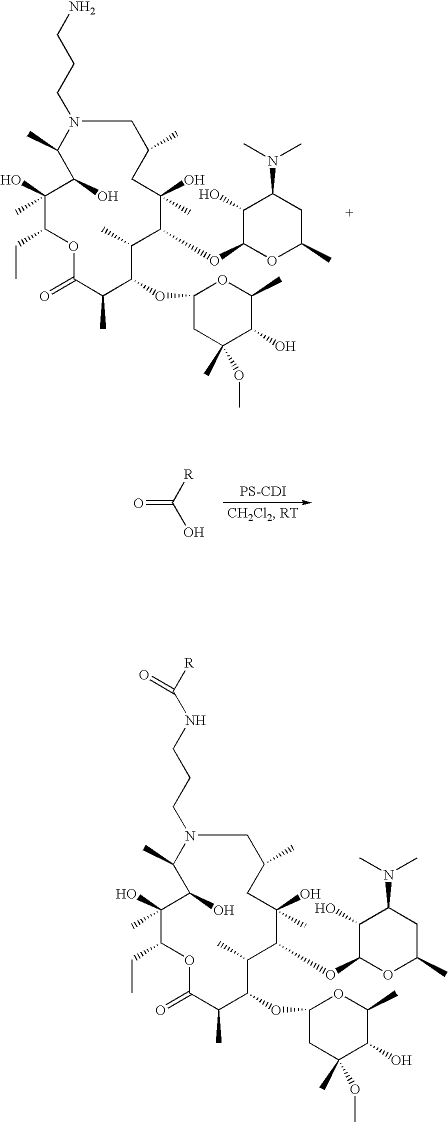

TABLE 5

| | | | | | Purity | |
|---|---|---|---|---|---|---|
| | | | | | % | |
| | | Starting | Isolated | MS | HPLC- | |
| | | acid | amide | (ES+) | MS | Yield |
| Example | R | (mg) | (mg) | m/z | area % | % |
| 58 | 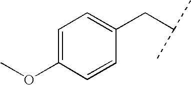 | 8.1 | 15.03 | 940.8[M + H]⁺,calcd.940.6 | 97.98 | 40.10 |
| 59 | 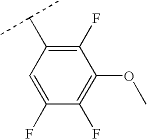 | 10.1 | 10.08 | 980.7[M + H]⁺,calcd.980.6 | 99.21 | 25.85 |
| 60 | 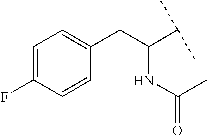 | 11.0 | 15.72 | 999.8[M + H]⁺,calcd.999.6 | 98.12 | 39.58 |
| 61 | 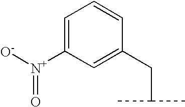 | 8.9 | 5.68 | 955.7[M + H]⁺,calcd.955.6 | 93.70 | 14.93 |
| 62 | 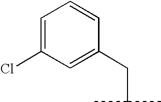 | 8.4 | 11.78 | 944.8[M + H]⁺,calcd.944.6 | 98.34 | 31.29 |
| 63 | 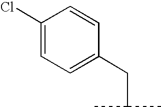 | 8.4 | 13.08 | 944.8[M + H]⁺,calcd.944.6 | 98.07 | 34.74 |
| 64 | 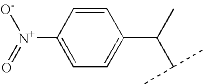 | 9.6 | 16.36 | 969.8[M + H]⁺,calcd.969.6 | 98.27 | 42.40 |
| 65 | 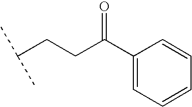 | 8.7 | 16.17 | 952.8[M + H]⁺,calcd.952.6 | 97.22 | 42.63 |
| 66 | 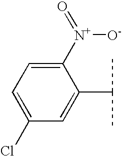 | 9.9 | 10.82 | 975.8[M + H]⁺,calcd.975.5 | 96.64 | 27.87 |
| 67 | 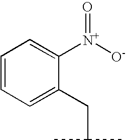 | 8.9 | 11.00 | 955.8[M + H]⁺,calcd.955.6 | 92.44 | 28.91 |
| 68 | 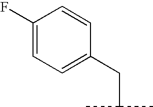 | 7.6 | 18.75 | 928.8[M + H]⁺,calcd.928.6 | 95.17 | 50.65 |
| 69 | 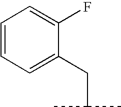 | 7.6 | 13.46 | 928.7[M + H]⁺,calcd.928.6 | 95.76 | 36.36 |
| 70 | 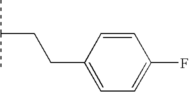 | 8.2 | 18.00 | 942.8[M + H]⁺,calcd.942.6 | 97.16 | 47.93 |
| 71 | 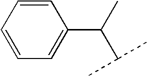 | 7.4 | 15.04 | 924.7[M + H]⁺,calcd.924.6 | 97.39 | 40.80 |
| 72 | 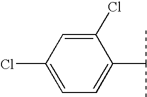 | 9.4 | 4.38 | 964.4[M + H]⁺,calcd.964.5 | 96.07 | 11.40 |
| 73 | 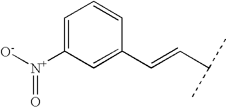 | 9.5 | 7.06 | 967.6[M + H]⁺,calcd.967.6 | 95.28 | 18.34 |
| 74 | 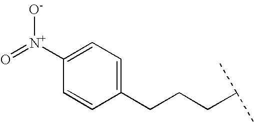 | 10.3 | 9.22 | 983.6[M + H]⁺,calcd.983.6 | 96.04 | 23.57 |
| 75 | 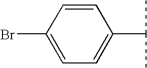 | 9.9 | 5.13 | 974.4[M + H]⁺,calcd.974.5 | 96.43 | 13.22 |

### Example 58

### 9a-[3-({[4-(Methyloxy)phenyl]acetyl}amino)propyl]-9-deoxo-9-dihydro-9a-aza-9a-homoerythromycin A Formiate Salt

### Example 59

### 9a-[3-({[2,4,5-Trifluoro-3-(methyloxy)phenyl]carbonyl}amino)propyl]-9-deoxo-9-dihydro-9a-aza-9a-homoerythromycin A Formiate Salt

### Example 60

### 9a-{3-[(N-Acetyl-4-fluorophenylalanyl)amino]propyl}-9-deoxo-9-dihydro-9a-aza-9a-homoerythromycin A Formiate Salt

### Example 61

### 9a-(3-{[(3-Nitrophenyl)acetyl]amino}propyl)-9-deoxo-9-dihydro-9a-aza-9a-homoerythromycin A Formiate Salt

### Example 62

### 9a-(3-{[(3-Chlorophenyl)acetyl]amino}propyl)-9-deoxo-9-dihydro-9a-aza-9a-homoerythromycin A Formiate Salt

### Example 63

### 9a-(3-{[(4-Chlorophenyl)acetyl]amino}propyl)-9-deoxo-9-dihydro-9a-aza-9a-homoerythromycin A Formiate Salt

### Example 64

### 9a-(3-{[(4-Nitrophenyl)acetyl]amino}propyl)-9-deoxo-9-dihydro-9a-aza-9a-homoerythromycin A Formiate Salt

### Example 65

### 9a-{3-[(4-Oxo-4-phenylbutanoyl)amino]propyl}-9-deoxo-9-dihydro-9a-aza-9a-homoerythromycin A Formiate Salt

### Example 66

### 9a-(3-{[(5-Chloro-2-nitrophenyl)carbonyl]amino}propyl)-9-deoxo-9-dihydro-9a-aza-9a-homoerythromycin A Formiate Salt

### Example 67

### 9a-(3-{[(2-Nitrophenyl)acetyl]amino}propyl)-9-deoxo-9-dihydro-9a-aza-9a-homoerythromycin A Formiate Salt

### Example 68

### 9a-(3-{[(4-Fluorophenyl)acetyl]amino}propyl)-9-deoxo-9-dihydro-9a-aza-9a-homoerythromycin A Formiate Salt

### Example 69

### 9a-(3-{[(2-Fluorophenyl)acetyl]amino}propyl)-9-deoxo-9-dihydro-9a-aza-9a-homoerythromycin A Formiate Salt

### Example 70

### 9a-(3-{[3-(4-Fluorophenyl)propanoyl]amino}propyl)-9-deoxo-9-dihydro-9a-aza-9a-homoerythromycin A Formiate Salt

### Example 71

### 9a-{3-[(2-phenylpropanoyl)amino]propyl}-9-deoxo-9-dihydro-9a-aza-9a-homoerythromycin A Formiate Salt

### Example 72

### 9a-(3-{[(2,4-Dichlorophenyl)carbonyl]amino}propyl)-9-deoxo-9-dihydro-9a-aza-9a-homoerythromycin A Formiate Salt

### Example 73

### 9a-(3-{[3-(3-Nitrophenyl)-2-propenyl]amino}propyl)-9-deoxo-9-dihydro-9a-aza-9a-homoerythromycin A Formiate Salt

### Example 74

### 9a-(3-{[4-(4-nitrophenyl)butanoyl]amino}propyl)-9-deoxo-9-dihydro-9a-aza-9a-homoerythromycin A Formiate Salt

### Example 75

### 9a-(3-{[(4-Bromophenyl)carbonyl]amino}propyl)-9-deoxo-9-dihydro-9a-aza-9a-homoerythromycin A Formiate Salt

### Example 76

### 9a-(3-{[(2E)-3-(Quinolin-3-yl)-2-propenoyl]amino}propyl)-9-deoxo-9-dihydro-9a-aza-9a-homoerythromycin A

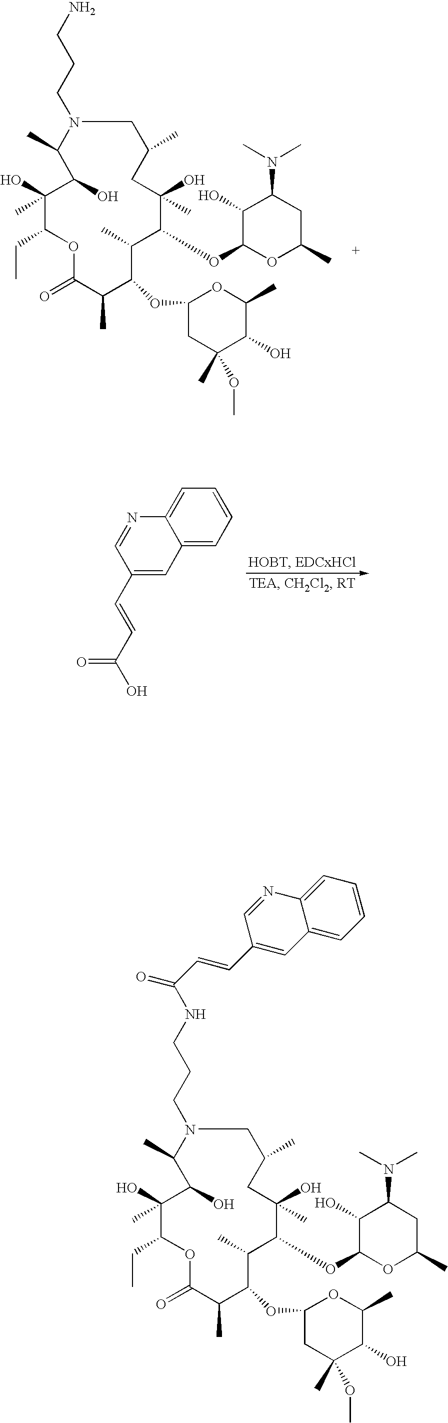

To a solution of (2E)-3-(3-quinolinyl)-2-propenoic acid, Intermediate 9 (9.4 mg, 0.047 mmol) in dry DCM (3.2 mL), TEA (0.73 mL, 0.47 mmol), HOBT (12.7 mg, 0.094 mmol), 9a-(γ-aminopropyl)-9a-aza-9-deoxo-9-dihydro-9a-homoerythromycin A (37.0 mg, 0.047 mmol) and EDCxHCl (36.0 mg, 0.188 mmol) were added. The reaction mixture was stirred at room temperature for 18 hours and than at 40° C. for 8 hours. After solvent was evaporated under reduced pressure the yellowish crude title product was obtained (96.0 mg) in form of base.

The purification was performed on preparative LC-MS (XTerra Prep RP₁₈ column, 5 μm, 19×100 mm) using gradient system for elution (0.1% HCOOH in H₂O/CH₃CN) in which HCOOH:CH₃CN was changed from 95:5 to 60:40 to give the title compound (13.64 mg, yield 28.47%) as formiate salt; MS (ES+) m/z 973.8 [M+H]⁺.

### Example 77

### 9a-{3-[(7-Chloro-quinolin-4-yl)amino]propyl]}-9-deoxo-9-dihydro-9a-aza-9a-homoerythromycin A Triacetate Salt

To a solution of 9a-{3-[(7-chloro-quinolin-4-yl)amino]propyl]}-9-deoxo-9-dihydro-9a-aza-9a-homoerythromycin A, Example 21A (1.1 g, 1.15 mmol) in propyl acetate (15 mL) glacial acetic acid (228 μL, 3.81 mmol) was added and stirred for 5 minutes. After addition of diisopropyl ether (5 mL) and n-hexane (50 mL) to the reaction mixture triacetate salt was precipitated. Precipitate was filtered off and dried under vacuum at room temperature giving the title compound as a white powder (1.1 g).

### Example 78

### 9a-{3-[(Quinolin-4-yl-methyl)amino]propyl}-9-deoxo-9-dihydro-9a-aza-9a-homoerythromycin A Diacetate Salt

To a solution of 9a-{3-[(Quinolin-4-yl-methyl)amino]propyl}-9-deoxo-9-dihydro-9a-aza-9a-homoerythromycin A, Example 14 (685 mg, 0.73 mmol) in DCM (1.5 mL) glacial acetic acid (84 μL, 1.47 mmol) was added. After addition of n-hexane (100 mL) diacetate salt was precipitated. Precipitate was filtered off and dried under vacuum for 1.5 hours at room temperature giving the title compound (528 mg).

### Example 79

### 9a-{3-[(7-Chloro-quinolin-4-yl)amino]propyl]}-3-O-decladinosyl-9-deoxo-9-dihydro-9a-aza-9a-homoerythromycin A Triacetate Salt

To a solution of 9a-{3-[(7-chloro-quinolin-4-yl)amino]propyl]}-3-O-decladinosyl-9-deoxo-9-dihydro-9a-aza-9a-homoerythromycin A, Example 23 (435 mg, 0.55 mmol) in ethyl acetate (2.2 mL) glacial acetic acid (94 μL, 1.64 mmol) was added. After addition of n-hexane (150 mL) triacetate salt was precipitated. Precipitate was filtered off and dried under vacuum for 1.5 hours at room temperature giving the title compound (401 mg).

### Example 80

### In Vitro Assay

The in vitro potency of the compounds has been compared with that of azithromycin. Using the methodology described in the In vitro screening protocol I or In vitro screening protocol II the compounds listed in the Table 6 were profiled for their antimalarial activity against four different P. falciparum parasites (D6, TM91C235, W2 and 3D7A) with different patterns of resistance. The IC₅₀ values of the tested compounds are provided as a range:

Key to Table
X = IC₅₀ in ng/mL

| | |
|---|---|
| A | X ≦ 100 |
| B | 100 < X ≦ 200 |
| C | 200 < X ≦ 1000 |
| D | 1000 < X ≦ 2500 |
| E | 2500 < X ≦ 2800 |
| F | 2800 < X ≦ 3500 |
| G | 3500 < X ≦ 5000 |
| H | 5000 < X ≦ 10000 |

TABLE 6

| | In vitro screening | | | In vitro screening | |
|---|---|---|---|---|---|
| | protocol I | | | protocol II | |
| Compound | D6 | TM91C235 | W2 | W2 | 3D7A |
| azithromycin | D | G | F | E | |
| Example 1 | B | A | A | | |
| Example 2 | C | B | B | | |
| Example 3 | G | D | C | | |
| Example 4 | B | A | A | | |
| Example 5 | C | B | B | | |
| Example 6 | F | D | C | | |
| Example 7 | A | A | A | | |
| Example 8 | A | A | A | A | B |
| Example 9 | A | A | A | | |
| Example 10 | C | C | C | | |
| Example 11 | G | F | C | | |
| Example 12 | C | C | C | | |
| Example 13 | G | D | C | | |
| Example 14 | B | B | B | | |
| Example 15 | B | C | B | C | C |
| Example 16 | B | A | A | | |
| Example 17 | B | A | A | | |
| Example 18 | D | D | C | | |
| Example 19 | B | A | A | | |
| Example 20 | C | A | A | | |
| Example 21A | A | A | A | | |
| Example 21B | B | A | B | | |
| Example 22 | A | A | A | | |
| Example 23 | A | A | A | | |
| Example 24 | B | C | B | | |
| Example 25 | D | C | D | | |
| Example 27 | C | A | C | | |
| Example 28 | C | A | C | | |
| Example 29 | C | A | C | | |
| Example 31 | C | A | B | | |
| Example 32 | C | A | B | | |
| Example 33 | B | A | C | | |
| Example 34 | E | C | D | | |
| Example 36 | D | D | D | | |
| Example 37 | C | C | C | | |
| Example 38 | C | C | C | | |
| Example 39 | C | C | C | | |
| Example 40 | C | B | B | | |
| Example 41 | B | A | A | | |
| Example 42 | B | A | A | | |
| Example 43 | B | A | A | | |
| Example 46 | C | C | C | | |
| Example 47 | — | — | — | D | D |
| Example 52 | C | A | A | | |
| Example 54 | | | | A | B |
| Example 55 | | | | A | C |
| Example 56 | | | | B | A |
| Example 58 | | | | C | G |
| Example 59 | | | | B | D |
| Example 60 | | | | D | H |
| Example 62 | | | | C | C |
| Example 63 | | | | C | C |
| Example 64 | | | | C | D |
| Example 65 | | | | D | G |
| Example 66 | | | | C | D |
| Example 67 | | | | D | H |
| Example 68 | | | | C | F |
| Example 69 | | | | C | G |
| Example 70 | | | | C | D |
| Example 76 | | | | C | C |
| Example 77 | | | | A | A |
| Example 78 | | | | A | C |
| Example 79 | | | | A | A |

In the Table an empty box means that compound was not tested against the stated strain.

1. (canceled)

2. A compound of Formula (Ia): 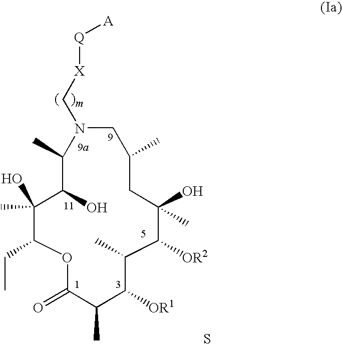
wherein
R¹ represents H or a α-L-cladinosyl group of formula (II) 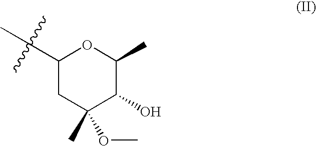
R² represents H or a β-D-desosaminyl group of formula (III) 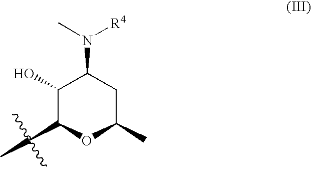
provided that when R² is H then R¹ is also H;
X represents NR³ or NHC(═O) or C(═O)NH;
R³ represents H or linear or branched C₁₋₄alkyl;
R⁴ represents H or linear or branched C₁₋₄alkyl;
Q represents
a) single bond,
b) C₁₋₄alkylene linear or branched which is unsubstituted or substituted,
c) C₂₋₄alkenylene;
A represents
a) aryl wherein aryl is mono-, bicyclic or tricyclic carbocyclic ring system having at least one aromatic ring which is unsubstituted or substituted by 1-4 groups selected from unsubstituted or substituted C₁₋₄ alkyl, unsubstituted or substituted C₃₋₆ cycloalkyl, halogen, OH, NO₂, C₁₋₄ alkyloxy, C₃₋₆ cycloalkyloxy, C₁₋₄ alkylamino, C₁₋₄ dialkylamino, C₃₋₆ cycloalkylamino; or
b) a 3-14 membered heterocycle, wherein heterocycle is a monocyclic, bicyclic or tricyclic ring any of which is saturated, unsaturated or aromatic containing 1 to 4 heteroatoms selected from nitrogen (unsubstituted or substituted by H or C₁₋₄ alkyl), oxygen and sulphur, unsubstituted or substituted on 1-3 ring carbon atoms by groups independently selected from unsubstituted or substituted C₁₋₄ alkyl, unsubstituted or substituted C₃₋₆ cycloalkyl, halogen, OH, NO₂, C₁₋₄ alkyloxy, C₃₋₆ cycloalkyloxy, C₁₋₄ alkylamino, C₁₋₄ dialkylamino, or C₃₋₆ cycloalkylamino;
m is an integer from 2 to 4;
or a pharmaceutically acceptable derivative thereof.
provided that when R² represents a β-D-desosaminyl group of formula (III),
X represents NR³,
R³ represents H or C₁₋₃alkyl,
m represents 2 or 3,
Q represents linear unsubstituted C₁₋₄alkylene and A represents unsubstituted or substituted phenyl or unsubstituted or substituted heteroaryl with five or six members containing from 1 to 3 atoms selected from nitrogen, oxygen and sulphur then R⁴ represents linear or branched C₄alkyl;
provided that A cannot represent a nonsteroidal subunit derived from a nonsteroidal anti-inflammatory drug (NSAID);

3. A compound as claimed in claim 2, wherein X represents NR³.

4. A compound as claimed in claim 3, wherein R³ is H.

5. A compound as claimed in claim 2, wherein X represents NHC(═O) or C(═O)NH.

6. A compound as claimed in claim 2, wherein Q represents single bond.

7. A compound as claimed in claim 2, wherein Q represents C₁₋₄alkylene.

8. A compound as claimed in claim 2, wherein Q represents C₂₋₄alkenylene.

9. A compound as claimed in claim 2, wherein A is a quinoline derived moiety: 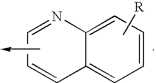
R is H or halogen.

10. A compound as claimed in claim 2, wherein A is a naphthalene derived moiety: 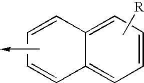
R is H or C₁₋₄alkyloxy and may be attached to either ring.

11. A compound as claimed in claim 2, wherein A is a pyridine derived moiety:

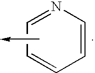

12. A compound as claimed in claim 2, wherein A is a phenyl derived moiety: 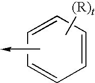
R is H, C₁₋₄alkyloxy, halogen or NO₂
t is 1 to 4.

13. A compound as claimed in claim 2, wherein A is a purine derived moiety:

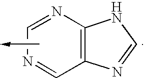

14. A compound as claimed in claim 2, wherein A is an oxazole derived moiety: 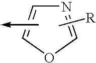
R is C₁₋₄alkyl.

15. A compound as claimed in claim 2, wherein R¹ represents H and R² represents a β-D-desosaminyl group of formula (III).

16. A compound as claimed in claim 2, wherein R¹ represents H and R² represents H.

17. A compound as claimed in claim 2, wherein m is 2 or 3.

18. A compound selected from the group consisting of:
9a-{3-[(quinolin-2-yl-methyl)amino]propyl}-9-deoxo-9-dihydro-9a-aza-9a-homoerythromycin A;
9a-{3-[(quinolin-2-yl-methyl)amino]propyl}-3-O-decladinosyl-9-deoxo-9-dihydro-9a-aza-9a-homoerythromycin A;
9a-{3-[(quinolin-2-yl-methyl)amino]propyl}-3-O-decladinosyl-5-O-dedesosaminyl-9-deoxo-9-dihydro-9a-aza-9a-homoerythromycin A;
9a-{3-[(quinolin-3-yl-methyl)amino]propyl}-9-deoxo-9-dihydro-9a-aza-9a-homoerythromycin A;
9a-{3-[(quinolin-3-yl-methyl)amino]propyl}-3-O-decladinosyl-9-deoxo-9-dihydro-9a-aza-9a-homoerythromycin A;
9a-{3-[(quinolin-3-yl-methyl)amino]propyl}-3-O-decladinosyl-5-O-dedesosaminyl-9-deoxo-9-dihydro-9a-aza-9a-homoerythromycin A;
9a-[3-({[2-(ethyloxy)-naphthalen-1-yl]methyl}amino)propyl]-9-deoxo-9-dihydro-9a-aza-9a-homoerythromycin A;
9a-{3-[(naphtalen-1-yl-methyl)amino]propyl}-9-deoxo-9-dihydro-9a-aza-9a-homoerythromycin A;
9a-{3-[(naphtalen-1-yl-methyl)amino]propyl}-3-O-decladinosyl-9-deoxo-9-dihydro-9a-aza-9a-homoerythromycin A;
9a-{3-[(quinolin-4-yl-methyl)amino]propyl}-9-deoxo-9-dihydro-9a-aza-9a-homoerythromycin A;
9a-{3-[(quinolin-4-yl-methyl)amino]propyl}-3-O-decladinosyl-9-deoxo-9-dihydro-9a-aza-9a-homoerythromycin A;
9a-{2-[(naphtalen-1-yl-methyl)amino]ethyl}-9-deoxo-9-dihydro-9a-aza-9a-homoerythromycin A;
9a-{3-[methyl-(naphtalen-1-yl-methyl)amino]propyl}-9-deoxo-9-dihydro-9a-aza-9a-homoerythromycin A;
9a-{3-[(7-chloro-quinolin-4-yl)amino]propyl]}-9-deoxo-9-dihydro-9a-aza-9a-homoerythromycin A;
9a-{3-[(7-chloro-quinolin-4-yl)amino]propyl]}-3′N-demethyl-9-deoxo-9-dihydro-9a-aza-9a-homoerythromycin A;
9a-[3-(quinolin-4-yl-amino)propyl]-9-deoxo-9-dihydro-9a-aza-9a-homoerythromycin A;
9a-{3-[(7-chloro-quinolin-4-yl)amino]propyl]}-3-O-decladinosyl-9-deoxo-9-dihydro-9a-aza-9a-homoerythromycin A;
9a-[3-(quinolin-4-yl-amino)propyl]-3-O-decladinosyl-9-deoxo-9-dihydro-9a-aza-9a-homoerythromycin A;
9a-[3-(quinolin-4-yl-amino)propyl]-3′-N-demethyl-9-deoxo-9-dihydro-9a-aza-9a-homoerythromycin A;
9a-[3-(1H-purin-6-yl-amino)propyl]-9-deoxo-9-dihydro-9a-aza-9a-homoerythromycin A;
9a-{3-[(3-phenylpropanoyl)amino]propyl}-9a-aza-9-deoxo-9-dihydro-9a-homoerythromycin A formiate salt;
9a-{3-[(4-phenylbutanoyl)amino]propyl}-9a-aza-9-deoxo-9-dihydro-9a-homoerythromycin A formiate salt;
9a-{3-[(naphtalen-1-yl-acetyl)amino]propyl}-9a-aza-9-deoxo-9-dihydro-9a-homoerythromycin A formiate salt;
9a-{3-[(phenylacetyl)amino]propyl}-9a-aza-9-deoxo-9-dihydro-9a-homoerythromycin A formiate salt;
9a-{3-[(5-phenylpentanoyl)amino]propyl}-9a-aza-9-deoxo-9-dihydro-9a-homoerythromycin A formiate salt;
9a-{3-[(naphtalen-2-yl-acetyl)amino]propyl}-9a-aza-9-deoxo-9-dihydro-9a-homoerythromycin A formiate salt;
9a-(3-{[(4-methyl-1,3-oxazol-5-yl)carbonyl]amino}propyl)-9a-aza-9-deoxo-9-dihydro-9a-homoerythromycin A formiate salt;
9a-(3-{[(4-methyl-1,3-oxazol-5-yl)carbonyl]amino}propyl)-3-O-decladinosyl-5-O-1-dedesosaminyl-9a-aza-9-deoxo-9-dihydro-9a-homoerythromycin A formiate salt;
9a-{3-[(naphtalen-1-yl-acetyl)amino]propyl}-3-O-decladinosyl-9a-aza-9-deoxo-9-dihydro-9a-homoerythromycin A formiate salt;
9a-[3-({(2S)-2-[6-(methyloxy)-naphthalen-2-yl]propanoyl}amino)propyl]-3-O-1-decladinosyl-9a-aza-9-deoxo-9-dihydro-9a-homoerythromycin A formiate salt;
9a-{1-[(phenylmethyl)amino]propanoyl}-9-deoxo-9-dihydro-9a-aza-9a-homoerythromycin A formiate salt;
9a-{1-[(2-phenylethyl)amino]propanoyl}-9-deoxo-9-dihydro-9a-aza-9a-homoerythromycin A formiate salt;
9a-{1-[(3-phenylpropyl)amino]propanoyl}-9-deoxo-9-dihydro-9a-aza-9a-homoerythromycin A formiate salt;
9a-{1-[(4-phenylbutyl)amino]propanoyl}-9-deoxo-9-dihydro-9a-aza-9a-homoerythromycin A formiate salt;
9a-{1-[(1S)-1-(1-naphthalenyl)ethyl]amino)propanoyl}-9-deoxo-9-dihydro-9a-aza-9a-homoerythromycin A formiate salt;
9a-{1-(2-naphthalenylmethyl)amino]propanoyl}-9-deoxo-9-dihydro-9a-aza-9a-homoerythromycin A formiate salt;
9a-{1-[(1S)-1-(1-naphthalenyl)ethyl]amino)propanoyl}-9-deoxo-9-dihydro-9a-aza-9a-homoerythromycin A diacetate salt;
9a-{1-[(1S)-1-(1-naphthalenyl)ethyl]amino)propanoyl}-9-deoxo-9-dihydro-9a-aza-9a-homoerythromycin A;
9a-[3-(quinolin-4-yl-amino)propyl]-9-deoxo-9-dihydro-9a-aza-9a-homoerythromycin A triacetate salt;
9a-{3-[(3-phenylpropanoyl)amino]propyl}-9a-aza-9-deoxo-9-dihydro-9a-homoerythromycin A;
9a-{3-[(3-phenylpropanoyl)amino]propyl}-9a-aza-9-deoxo-9-dihydro-9a-homoerythromycin A diacetate salt;
9a-{1-[(3-phenylpropyl)amino]propanoyl}-9-deoxo-9-dihydro-9a-aza-9a-homoerythromycin A;
9a-{1-[(3-phenylpropyl)amino]propanoyl}-9-deoxo-9-dihydro-9a-aza-9a-homoerythromycin A diacetate salt;
9a-{3-[(naphtalen-2-yl-acetyl)amino]propyl}-9a-aza-9-deoxo-9-dihydro-9a-homoerythromycin A;
9a-{3-[(naphtalen-2-yl-acetyl)amino]propyl}-9a-aza-9-deoxo-9-dihydro-9a-homoerythromycin A diacetate salt;
9a-{3-[(quinolin-4-yl-methyl)amino]propyl}-3-O-decladinosyl-9-deoxo-9-dihydro-9a-aza-9a-homoerythromycin A diacetate salt;
9a-{3-[(naphtalen-2-yl-methyl)amino]propyl}-9-deoxo-9-dihydro-9a-aza-9a-homoerythromycin A
9a-{3-[(1,2,3,4-tetrahydro-quinolin-4-yl-methyl)amino]propyl}-9-deoxo-9-dihydro-9a-aza-9a-homoerythromycin A;
9a-{3-[(7-chloro-quinolin-4-yl)amino]propyl]}-3-O-decladinosyl-5-O-dedesosaminyl-9-deoxo-9-dihydro-9a-aza-9a-homoerythromycin A;
9a-(3-{[3-(quinolin-4-yl)propanoyl]amino}propyl)-9-deoxo-9-dihydro-9a-aza-9a-homoerythromycin A;
9a-[3-({[4-(methyloxy)phenyl]acetyl}amino)propyl]-9-deoxo-9-dihydro-9a-aza-9a-homoerythromycin A formiate salt;
9a-[3-({[2,4,5-trifluoro-3-(methyloxy)phenyl]carbonyl}amino)propyl]-9-deoxo-9-dihydro-9a-aza-9a-homoerythromycin A formiate salt;
9a-{3-[(N-acetyl-4-fluorophenylalanyl)amino]propyl}-9-deoxo-9-dihydro-9a-aza-9a-homoerythromycin A formiate salt;
9a-(3-{[(3-nitrophenyl)acetyl]amino}propyl)-9-deoxo-9-dihydro-9a-aza-9a-homoerythromycin A formiate salt;
9a-(3-{[(3-chlorophenyl)acetyl]amino}propyl)-9-deoxo-9-dihydro-9a-aza-9a-homoerythromycin A formiate salt;
9a-(3-{[(4-chlorophenyl)acetyl]amino}propyl)-9-deoxo-9-dihydro-9a-aza-9a-homoerythromycin A formiate salt;
9a-(3-{[(4-nitrophenyl)acetyl]amino}propyl)-9-deoxo-9-dihydro-9a-aza-9a-homoerythromycin A formiate salt;
9a-{3-[(4-oxo-4-phenylbutanoyl)amino]propyl}-9-deoxo-9-dihydro-9a-aza-9a-homoerythromycin A formiate salt;
9a-(3-{[(5-chloro-2-nitrophenyl)carbonyl]amino}propyl)-9-deoxo-9-dihydro-9a-aza-9a-homoerythromycin A formiate salt;
9a-(3-{[(2-nitrophenyl)acetyl]amino}propyl)-9-deoxo-9-dihydro-9a-aza-9a-homoerythromycin A formiate salt;
9a-(3-{[(4-fluorophenyl)acetyl]amino}propyl)-9-deoxo-9-dihydro-9a-aza-9a-homoerythromycin A formiate salt;
9a-(3-{[(2-fluorophenyl)acetyl]amino}propyl)-9-deoxo-9-dihydro-9a-aza-9a-homoerythromycin A formiate salt;
9a-(3-{[3-(4-fluorophenyl)propanoyl]amino}propyl)-9-deoxo-9-dihydro-9a-aza-9a-homoerythromycin A formiate salt;
9a-{3-[(2-phenylpropanoyl)amino]propyl}-9-deoxo-9-dihydro-9a-aza-9a-homoerythromycin A formiate salt;
9a-(3-{[(2,4-dichlorophenyl)carbonyl]amino}propyl)-9-deoxo-9-dihydro-9a-aza-9a-homoerythromycin A formiate salt;
9a-(3-{[3-(3-nitrophenyl)-2-propenoyl]amino}propyl)-9-deoxo-9-dihydro-9a-aza-9a-homoerythromycin A formiate salt;
9a-(3-{[4-(4-nitrophenyl)butanoyl]amino}propyl)-9-deoxo-9-dihydro-9a-aza-9a-homoerythromycin A formiate salt;
9a-(3-{[(4-bromophenyl)carbonyl]amino}propyl)-9-deoxo-9-dihydro-9a-aza-9a-homoerythromycin A formiate salt;
9a-(3-{[(2E)-3-(quinolin-3-yl)-2-propenoyl]amino}propyl)-9-deoxo-9-dihydro-9a-aza-9a-homoerythromycin A;
9a-{3-[(7-chloro-quinolin-4-yl)amino]propyl]}-9-deoxo-9-dihydro-9a-aza-9a-homoerythromycin A triacetate salt;
9a-{3-[(quinolin-4-yl-methyl)amino]propyl}-9-deoxo-9-dihydro-9a-aza-9a-homoerythromycin A diacetate salt;
9a-{3-[(7-chloro-quinolin-4-yl)amino]propyl]}-3-O-decladinosyl-9-deoxo-9-dihydro-9a-aza-9a-homoerythromycin A triacetate salt; and
pharmaceutically acceptable derivatives thereof.

19. A compound of according to claim 2 selected from the group consisting of:
9a-{3-[(pyridine-4-yl-methyl)amino]propyl}-9-deoxo-9-dihydro-9a-aza-9a-homoerythromycin A;
9a-{3-[(pyridine-4-yl-methyl)amino]propyl}-3-O-decladinosyl-9-deoxo-9-dihydro-9a-aza-9a-homoerythromycin A;
9a-{3-[(pyridine-3-yl-methyl)amino]propyl}-9-deoxo-9-dihydro-9a-aza-9a-homoerythromycin A;
9a-{3-[(pyridine-3-yl-methyl)amino]propyl}-3-O-decladinosyl-9-deoxo-9-dihydro-9a-aza-9a-homoerythromycin A;
9a-{3-[(pyridin-2-yl-methyl)amino]propyl}-9-deoxo-9-dihydro-9a-aza-9a-homoerythromycin A;
9a-{3-[(3-phenylpropyl)amino]propyl}-9-deoxo-9-dihydro-9a-aza-9a-homoerythromycin A;
9a-{3-[(2-phenylethyl)amino]propyl}-9-deoxo-9-dihydro-9a-aza-9a-homoerythromycin A; and
pharmaceutically acceptable derivatives thereof.

20. (canceled)

21. A process for the preparation of the compound of Formula (Ia) as claimed in claim 2,
which process comprises one of:
a) reacting a compound of Formula (IV) 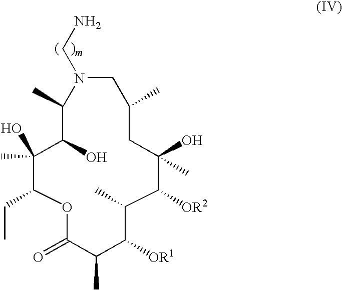
wherein R¹, R² and m have the meaning as defined in claim 2 with aldehyde of Formula (V) 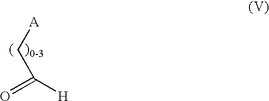
using suitable reducing agent to produce a compound of Formula (Ia) wherein X is NR³, R³ is hydrogen, Q is C₁₋₄alkylene and R¹, R², R⁴ and m have the meanings defined in claim 2; or
b) reacting a compound of Formula (Ia) wherein X is NR³ and R³ is hydrogen by reductive alkylation with aldehyde of formula (VIa) or (VIb) using suitable reducing agents 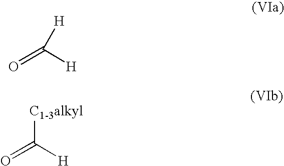
to produce a compound of Formula (I) wherein X is NR³ and R³ is C₁-C₄ alkyl and R¹, R², R⁴, m and Q have the meanings defined in claim 2; or
c) reacting a compound of Formula (IV) with compound of formula A-L (VII), wherein L is suitable leaving group to produce compound of Formula (Ia), wherein Q is a bond, X is NR³, R¹, R², R³, R⁴ and m have the meanings defined in claim 2; or
d) reacting a compound of Formula (IV) with suitable activated derivative of carboxylic acid of Formula HOC(O)(CH₂)₁₋₄A (VIII) selected from acyl halide, mixed anhydride and activated ester, or with the carboxylic acid of Formula (VIII) in the presence of carbodiimides selected from dicyclohexylcarbodiimide, 1,8-diazabicyclo[5.4.0.]undec-7-ene and 1-(3-dimethylaminopropyl)-3-ethylcarbodiimide to produce compound of Formula (Ia), wherein X is NHC(O) and R¹, R², R⁴, m and Q have the meanings defined in claim 2; or
e) reacting a compound of Formula (IX) with amine of Formula A-(CH₂)ₙ—NH₂ (X) in the presence of EDC, organic or inorganic base selected from 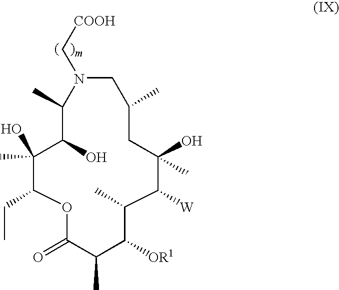
dimethylaminopyridine, triethylamine or DBU, sodium hydroxide, lithium hydroxide and potassium hydroxyide to produce compound of Formula (Ia), wherein X is C(O)NH and R¹, R², R⁴, m and Q have the meanings defined in claim 2.

22. A method for the therapeutic and/or prophylactic treatment of malaria in a subject in need of such treatment comprising administering to the subject a therapeutically effective amount of a compound of Formula (I) or a pharmaceutically acceptable derivative thereof: 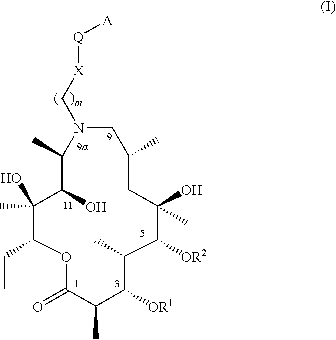
wherein
R¹ represents H or a α-L-cladinosyl group of formula (II) 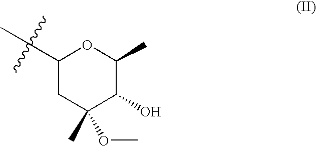
R² represents H or a β-D-desosaminyl group of formula (III) 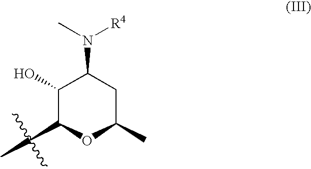
provided that when R² is H then R¹ is also H:
X represents NR³ or NHC(═O) or C(═O)NH;
R³ represents H or linear or branched C₁₋₄alkyl;
R⁴ represents H or linear or branched C₁₋₄alkyl;
Q represents
a) single bond,
b) C₁₋₄alkylene linear or branched which is unsubstituted or substituted,
c) C₂₋₄alkenylene;
A represents
a) aryl wherein aryl is mono-, bicyclic or tricyclic carbocyclic ring system having at least one aromatic ring which is unsubstituted or substituted by 1-4 groups selected from unsubstituted or substituted C₁₋₄ alkyl, unsubstituted or substituted C₃₋₆, cycloalkyl, halogen, OH, NO₂, C₁₋₄ alkyloxy, C₃₋₆ cycloalkyloxy, C₁₋₄ alkylamino, C₁₋₄ dialkylamino, C₃₋₆ cycloalkylamino; or
b) a 3-14 membered heterocycle, wherein heterocycle is a monocyclic, bicyclic or tricyclic ring any of which is saturated, unsaturated or aromatic containing 1 to 4 heteroatoms selected from nitrogen (unsubstituted or substituted by H or C₁₋₄ alkyl), oxygen and sulphur, unsubstituted or substituted on 1-3 ring carbon atoms by groups independently selected from unsubstituted or substituted C₁₋₄ alkyl, unsubstituted or substituted C₃₋₆ cycloalkyl, halogen, OH, NO₂, C₁₋₄ alkyloxy, C₃₋₆ cycloalkyloxy. C₁₋₄ alkylamino, C₁₋₄ dialkylamino, or C₃₋₆ cycloalkylamino;
m is an integer from 2 to 4.

23. The method of claim 22, wherein the subject has been infected with Plasmodium falciparum, Plasmodium vivax, Plasmodium ovale or Plasmodium malariae.

24. A pharmaceutical composition comprising a compound as claimed in claim 2, or a pharmaceutically acceptable derivative salt thereof, in association with a pharmaceutically acceptable excipient, diluent and/or carrier.

25. (canceled)

26. The method according to claim 22 wherein the compound of Formula (I) is selected from the group consisting of:
9a-{3-[(quinolin-2-yl-methyl)amino]propyl}-9-deoxo-9-dihydro-9a-aza-9a-homoerythromycin A;
9a-{3-[(quinolin-2-yl-methyl)amino]propyl}-3-O-decladinosyl-9-deoxo-9-dihydro-9a-aza-9a-homoerythromycin A;
9a-{3-[(quinolin-2-yl-methyl)amino]propyl}-3-O-decladinosyl-5-O-dedesosaminyl-9-deoxo-9-dihydro-9a-aza-9a-homoerythromycin A;
9a-{3-[(quinolin-3-yl-methyl)amino]propyl}-9-deoxo-9-dihydro-9a-aza-9a-homoerythromycin A;
9a-{3-[(quinolin-3-yl-methyl)amino]propyl}-3-O-decladinosyl-9-deoxo-9-dihydro-9a-aza-9a-homoerythromycin A;
9a-{3-[(quinolin-3-yl-methyl)amino]propyl}-3-O-decladinosyl-5-O-dedesosaminyl-9-deoxo-9-dihydro-9a-aza-9a-homoerythromycin A;
9a-[3-({[2-(ethyloxy)-naphthalen-1-yl]methyl}amino)propyl]-9-deoxo-9-dihydro-9a-aza-9a-homoerythromycin A;
9a-{3-[(naphtalen-1-yl-methyl)amino]propyl}-9-deoxo-9-dihydro-9a-aza-9a-homoerythromycin A;
9a-{3-[(naphtalen-1-yl-methyl)amino]propyl}-3-O-decladinosyl-9-deoxo-9-dihydro-9a-aza-9a-homoerythromycin A;
9a-{3-[(quinolin-4-yl-methyl)amino]propyl}-9-deoxo-9-dihydro-9a-aza-9a-homoerythromycin A;
9a-{3-[(quinolin-4-yl-methyl)amino]propyl}-3-decladinosyl-9-deoxo-9-dihydro-9a-aza-9a-homoerythromycin A;
9a-{2-[(naphtalen-1-yl-methyl)amino]ethyl}-9-deoxo-9-dihydro-9a-aza-9a-homoerythromycin A;
9a-{3-[methyl-(naphtalen-1-yl-methyl)amino]propyl}-9-deoxo-9-dihydro-9a-aza-9a-homoerythromycin A;
9a-{3-[(7-chloro-quinolin-4-yl)amino]propyl]}-9-deoxo-9-dihydro-9a-aza-9a-homoerythromycin A;
9a-{3-[(7-chloro-quinolin-4-yl)amino]propyl]}-3′N-demethyl-9-deoxo-9-dihydro-9a-aza-9a-homoerythromycin A;
9a-[3-(quinolin-4-yl-amino)propyl]-9-deoxo-9-dihydro-9a-aza-9a-homoerythromycin A;
9a-{3-[(7-chloro-quinolin-4-yl)amino]propyl]}-3-O-decladinosyl-9-deoxo-9-dihydro-9a-aza-9a-homoerythromycin A;
9a-[3-(quinolin-4-yl-amino)propyl]-3-O-decladinosyl-9-deoxo-9-dihydro-9a-aza-9a-homoerythromycin A;
9a-[3-(quinolin-4-yl-amino)propyl]-3′-N-demethyl-9-deoxo-9-dihydro-9a-aza-9a-homoerythromycin A;
9a-[3-(1H-purin-6-yl-amino)propyl]-9-deoxo-9-dihydro-9a-aza-9a-homoerythromycin A;
9a-{3-[(3-phenylpropanoyl)amino]propyl}-9a-aza-9-deoxo-9-dihydro-9a-homoerythromycin A formiate salt;
9a-{3-[(4-phenylbutanoyl)amino]propyl}-9a-aza-9-deoxo-9-dihydro-9a-homoerythromycin A formiate salt;
9a-{3-[(naphtalen-1-yl-acetyl)amino]propyl}-9a-aza-9-deoxo-9-dihydro-9a-homoerythromycin A formiate salt;
9a-{3-[(phenylacetyl)amino]propyl}-9a-aza-9-deoxo-9-dihydro-9a-homoerythromycin A formiate salt;
9a-{3-[(5-phenylpentanoyl)amino]propyl}-9a-aza-9-deoxo-9-dihydro-9a-homoerythromycin A formiate salt;
9a-{3-[(naphtalen-2-yl-acetyl)amino]propyl}-9a-aza-9-deoxo-9-dihydro-9a-homoerythromycin A formiate salt;
9a-(3-{[(4-methyl-1,3-oxazol-5-yl)carbonyl]amino}propyl)-9a-aza-9-deoxo-9-dihydro-9a-homoerythromycin A formiate salt;
9a-(3-{[(4-methyl-1,3-oxazol-5-yl)carbonyl]amino}propyl)-3-O-decladinosyl-5-O-1-dedesosaminyl-9a-aza-9-deoxo-9-dihydro-9a-homoerythromycin A formiate salt;
9a-{3-[(naphtalen-1-yl-acetyl)amino]propyl}-3-O-decladinosyl-9a-aza-9-deoxo-9-dihydro-9a-homoerythromycin A formiate salt;
9a-[3-({(2S)-2-[6-(methyloxy)-naphthalen-2-yl]propanoyl}amino)propyl]-3-O-decladinosyl-9a-aza-9-deoxo-9-dihydro-9a-homoerythromycin A formiate salt;
9a-{1-[(phenylmethyl)amino]propanoyl}-9-deoxo-9-dihydro-9a-aza-9a-homoerythromycin A formiate salt;
9a-{1-[(2-phenylethyl)amino]propanoyl}-9-deoxo-9-dihydro-9a-aza-9a-homoerythromycin A formiate salt;
9a-{1-[(3-phenylpropyl)amino]propanoyl}-9-deoxo-9-dihydro-9a-aza-9a-homoerythromycin A formiate salt;
9a-{1-[(4-phenylbutyl)amino]propanoyl}-9-deoxo-9-dihydro-9a-aza-9a-homoerythromycin A formiate salt;
9a-{1-[(1S)-1-(1-naphthalenyl)ethyl]amino)propanoyl}-9-deoxo-9-dihydro-9a-aza-9a-homoerythromycin A formiate salt;
9a-{1-(2-naphthalenylmethyl)amino]propanoyl}-9-deoxo-9-dihydro-9a-aza-9a-homoerythromycin A formiate salt;
9a-{1-[(1S)-1-(1-naphthalenyl)ethyl]amino)propanoyl}-9-deoxo-9-dihydro-9a-aza-9a-homoerythromycin A diacetate salt;
9a-{1-[(1S)-1-(1-naphthalenyl)ethyl]amino)propanoyl}-9-deoxo-9-dihydro-9a-aza-9a-homoerythromycin A;
9a-[3-(quinolin-4-yl-amino)propyl]-9-deoxo-9-dihydro-9a-aza-9a-homoerythromycin A triacetate salt;
9a-{3-[(3-phenylpropanoyl)amino]propyl}-9a-aza-9-deoxo-9-dihydro-9a-homoerythromycin A;
9a-{3-[(3-phenylpropanoyl)amino]propyl}-9a-aza-9-deoxo-9-dihydro-9a-homoerythromycin A diacetate salt;
9a-{1-[(3-phenylpropyl)amino]propanoyl}-9-deoxo-9-dihydro-9a-aza-9a-homoerythromycin A;
9a-{1-[(3-phenylpropyl)amino]propanoyl}-9-deoxo-9-dihydro-9a-aza-9a-homoerythromycin A diacetate salt;
9a-{3-[(naphtalen-2-yl-acetyl)amino]propyl}-9a-aza-9-deoxo-9-dihydro-9a-homoerythromycin A;
9a-{3-[(naphtalen-2-yl-acetyl)amino]propyl}-9a-aza-9-deoxo-9-dihydro-9a-homoerythromycin A diacetate salt;
9a-{3-[(quinolin-4-yl-methyl)amino]propyl}-3-O-decladinosyl-9-deoxo-9-dihydro-9a-aza-9a-homoerythromycin A diacetate salt;
9a-{3-[(naphtalen-2-yl-methyl)amino]propyl}-9-deoxo-9-dihydro-9a-aza-9a-homoerythromycin A;
9a-{3-[(1,2,3,4-tetrahydro-quinolin-4-yl-methyl)amino]propyl}-9-deoxo-9-dihydro-9a-aza-9a-homoerythromycin A;
9a-{3-[(7-chloro-quinolin-4-yl)amino]propyl]}-3-O-decladinosyl-5-O-dedesosaminyl-9-deoxo-9-dihydro-9a-aza-9a-homoerythromycin A;
9a-(3-{[3-(quinolin-4-yl)propanoyl]amino}propyl)-9-deoxo-9-dihydro-9a-aza-9a-homoerythromycin A;
9a-[3-({[4-(methyloxy)phenyl]acetyl}amino)propyl]-9-deoxo-9-dihydro-9a-aza-9a-homoerythromycin A formiate salt;
9a-[3-({[2,4,5-trifluoro-3-(methyloxy)phenyl]carbonyl}amino)propyl]-9-deoxo-9-dihydro-9a-aza-9a-homoerythromycin A formiate salt;
9a-{3-[(N-acetyl-4-fluorophenylalanyl)amino]propyl}-9-deoxo-9-dihydro-9a-aza-9a-homoerythromycin A formiate salt;
9a-(3-{[(3-nitrophenyl)acetyl]amino}propyl)-9-deoxo-9-dihydro-9a-aza-9a-homoerythromycin A formiate salt;
9a-(3-{[(3-chlorophenyl)acetyl]amino}propyl)-9-deoxo-9-dihydro-9a-aza-9a-homoerythromycin A formiate salt;
9a-(3-{[(4-chlorophenyl)acetyl]amino}propyl)-9-deoxo-9-dihydro-9a-aza-9a-homoerythromycin A formiate salt;
9a-(3-{[(4-nitrophenyl)acetyl]amino}propyl)-9-deoxo-9-dihydro-9a-aza-9a-homoerythromycin A formiate salt;
9a-{3-[(4-oxo-4-phenylbutanoyl)amino]propyl}-9-deoxo-9-dihydro-9a-aza-9a-homoerythromycin A formiate salt;
9a-(3-{[(5-chloro-2-nitrophenyl)carbonyl]amino}propyl)-9-deoxo-9-dihydro-9a-aza-9a-homoerythromycin A formiate salt;
9a-(3-{[(2-nitrophenyl)acetyl]amino}propyl)-9-deoxo-9-dihydro-9a-aza-9a-homoerythromycin A formiate salt;
9a-(3-{[(4-fluorophenyl)acetyl]amino}propyl)-9-deoxo-9-dihydro-9a-aza-9a-homoerythromycin A formiate salt;
9a-(3-{[(2-fluorophenyl)acetyl]amino}propyl)-9-deoxo-9-dihydro-9a-aza-9a-homoerythromycin A formiate salt;
9a-(3-{[3-(4-fluorophenyl)propanoyl]amino}propyl)-9-deoxo-9-dihydro-9a-aza-9a-homoerythromycin A formiate salt;
9a-{3-[(2-phenylpropanoyl)amino]propyl}-9-deoxo-9-dihydro-9a-aza-9a-homoerythromycin A formiate salt;
9a-(3-{[(2,4-dichlorophenyl)carbonyl]amino}propyl)-9-deoxo-9-dihydro-9a-aza-9a-homoerythromycin A formiate salt;
9a-(3-{[3-(3-nitrophenyl)-2-propenoyl]amino}propyl)-9-deoxo-9-dihydro-9a-aza-9a-homoerythromycin A formiate salt;
9a-(3-{[4-(4-nitrophenyl)butanoyl]amino}propyl)-9-deoxo-9-dihydro-9a-aza-9a-homoerythromycin A formiate salt;
9a-(3-{[(4-bromophenyl)carbonyl]amino}propyl)-9-deoxo-9-dihydro-9a-aza-9a-homoerythromycin A formiate salt;
9a-(3-{[(2E)-3-(quinolin-3-yl)-2-propenoyl]amino}propyl)-9-deoxo-9-dihydro-9a-aza-9a-homoerythromycin A;
9a-{3-[(7-chloro-quinolin-4-yl)amino]propyl]}-9-deoxo-9-dihydro-9a-aza-9a-homoerythromycin A triacetate salt;
9a-{3-[(quinolin-4-yl-methyl)amino]propyl}-9-deoxo-9-dihydro-9a-aza-9a-homoerythromycin A diacetate salt; and
9a-{3-[(7-chloro-quinolin-4-yl)amino]propyl]}-3-O-decladinosyl-9-deoxo-9-dihydro-9a-aza-9a-homoerythromycin A triacetate salt.
